(19) United States
(12) Patent Application Publication
(10) Pub. No.: US 2023/0183627 A1
(43) Pub. Date: Jun. 15, 2023

(54) BIOMANUFACTURING SYSTEMS AND METHODS FOR PRODUCING ORGANIC PRODUCTS FROM RECOMBINANT MICROORGANISMS

(71) Applicant: CEMVITA FACTORY, INC., Houston, TX (US)

(72) Inventors: Tahereh KARIMI, Houston, TX (US); Truong Huu NGUYEN, Houston, TX (US); MohammadMatin HANIFZADEH, Tempe, AZ (US); Samantha COOK ALBRIGHT, Houston, TX (US); Miguel Eugenio CUEVA, Houston, TX (US)

(73) Assignee: CEMVITA FACTORY, INC., Houston, TX (US)

(21) Appl. No.: 17/912,242

(22) PCT Filed: Mar. 19, 2021
(86) PCT No.: PCT/US2021/023332

§ 371 Date: Oct. 4, 2022

Related U.S. Application Data
(60) Provisional application No. 63/052,664, filed on Jul. 16, 2020.
(60) Provisional application No. 62/992,689, filed on Mar. 20, 2020.

(51) Int. Cl. C12M 1/00; C12M 1/107; C12P 21/00; C12P 5/02

(52) U.S. Cl. CPC C12M 23/58; C12M 21/04; C12P 21/00; C12P 5/026

(57) ABSTRACT

The present disclosure relates to biomanufacturing systems for producing an organic product. The present disclosure relates to recombinant microorganisms having an improved organic substrate producing ability, and to recombinant microorganisms having an improved organic product producing ability. A benefit of the systems and recombinant microorganisms disclosed herein can include an ability to separately produce an organic product and an organic substrate that generates a culture impurity during its production. The present disclosure relates to methods of producing an organic product using biomanufacturing systems and recombinant microorganisms disclosed herein.

### TECHNICAL FIELD

The present disclosure relates to biomanufacturing systems for producing an organic product. The present disclosure relates to recombinant microorganisms having an improved organic substrate producing ability, and to recombinant microorganisms having an improved organic product producing ability. A benefit of the systems and recombinant microorganisms disclosed herein can include an ability to separately and safely produce an organic product and an organic substrate that generates a culture impurity during its production. The present disclosure relates to methods of producing an organic product using biomanufacturing systems and recombinant microorganisms disclosed herein. A benefit of the methods disclosed herein can include the increased production of one or more organic products from microbial cultures. A benefit of the methods herein can be an ability to produce an organic product containing a reduced amount of byproducts or a culture impurity. An additional benefit can be the use of carbon dioxide to produce bio-ethylene useful as a feedstock for the production of plastics, textiles, and chemical materials, and for use in other applications. Another benefit is the use of carbon dioxide for production of alkenes, alkanes, polyenes, and alcohols. Another benefit of this method includes avoiding coproduction of oxygen and volatile organic compounds.

Another benefit of the methods and systems disclosed herein can include reduction of excess carbon dioxide from the environment.

### BACKGROUND

The increased demand for power worldwide has led to an excess of carbon dioxide from burning fossil fuels such as oil and gas, contributing substantially to what many are calling a global warming crisis. Industry is so desperate to prevent carbon dioxide from entering the atmosphere that they have resorted to sequestering carbon dioxide from exhaust streams and the atmosphere. They then store the carbon dioxide in subterranean environments. However, all current known methods just remove carbon dioxide from the atmosphere by storing it under ground. They do not actually convert the carbon dioxide back into any other useful material.

The limited supply of petroleum and its harmful effects on the environment have prompted developments in renewable sources of fuels and chemicals. Technologies that convert biomass and captured carbon dioxide into new products, such as biofuels, can help to reduce oil imports and carbon dioxide emissions. There is a growing interest in producing value-added fuels and other useful organic products from organic waste using biological processes. Among these organic products, ethylene is the most widely produced organic compound in the world, useful in a broad spectrum of industries including plastics, solvents, and textiles. Ethylene is currently produced by steam cracking fossil fuels or dehydrogenating ethane. With millions of metric tons of ethylene being produced each year, however, more than enough carbon dioxide is produced by such processes to greatly contribute to the global carbon footprint. Producing ethylene through renewable methods would accordingly help to meet the huge demand from the energy and chemical industries, while also helping to protect the environment.

Since ethylene is a potentially renewable feedstock, there has been a great deal of interest in developing technologies to produce ethylene from renewable sources, such as carbon dioxide and biomass. Bio-ethylene is currently produced using ethanol derived from corn or sugar cane. A variety of microbes, including bacteria and fungi, naturally produce ethylene in small amounts. Heterologous expression of an ethylene producing enzyme has been demonstrated in several microbial species, where the hosts have been able to utilize a variety of carbon sources, including lignocellulose and carbon dioxide.

Based on modern history, it is fair to say that excess carbon dioxide in the atmosphere, as well as other organic waste, will not be reduced until it becomes profitable to reduce them. There remains a need for improvements in microbial biomanufacturing systems and processes, in order to produce useful organic products such as ethylene at a commercial scale. There remains a need to produce hydrocarbons through more efficient renewable technologies. There remains a need to remove excess carbon dioxide from the atmosphere. There remains a need for improved methods to safely produce ethylene from a renewable feedstock for industrial and commercial applications.

### SUMMARY

Embodiments herein are directed to biomanufacturing systems for producing an organic product. In various embodiments, the system includes at least one bioreactor culture vessel. In various embodiments, the at least one bioreactor culture vessel contains an organic substrate culture solution, wherein the organic substrate culture solution contains a first recombinant microorganism. In various embodiments, the first recombinant microorganism has an improved organic substrate producing ability, expresses at least one organic substrate forming recombinant enzyme by expressing at least one non-native organic substrate forming enzyme nucleotide sequence, is capable of utilizing a carbon source to produce the organic substrate, and produces at least one organic substrate culture impurity, including at least one of a volatile gas, a liquid, and a solid. In various embodiments, the at least one bioreactor culture vessel contains an organic product culture solution, wherein the organic product culture solution contains a second recombinant microorganism. In various embodiments, the second recombinant microorganism has an improved organic product producing ability, expresses at least one organic product forming enzyme by expressing at least one non-native organic product forming enzyme nucleotide sequence, and is capable of utilizing the organic substrate to produce the at least one organic product.

In certain embodiments, the at least one bioreactor culture vessel includes a first bioreactor culture vessel including a carbon source inlet, a volatile gas outlet, and a power source; and a second bioreactor culture vessel including a fluid flow path connected to and between the first bioreactor culture vessel and the second bioreactor culture vessel, and an organic product outlet. In such embodiments, the first bioreactor culture vessel includes the organic substrate culture solution, and the second bioreactor culture vessel includes the organic product culture solution. In certain embodiments, the first bioreactor culture vessel or the second bioreactor culture vessel further includes a biomass collection port. In certain embodiments, the power source includes sunlight, a solar power source, an electrical power source, or a combination thereof. In certain embodiments, the system further includes a carbonation unit, an amine stripper, an amine scrubber, a catalytic converter, a condenser, a compressor, a caustic tower, a dryer, or combinations thereof. In certain embodiments, the second bioreactor culture vessel further includes a carbon source inlet, a volatile gas outlet, a power source, or a combination thereof.

In certain embodiments, the carbon source includes carbon dioxide, carbon monoxide, an n-alkane, ethanol, a vegetable oil, glycerol, glucose, sucrose, a monosaccharide, a disaccharide, a polysaccharide, or a combination thereof. In certain embodiments, the volatile gas includes oxygen, methane, or a combination thereof. In certain embodiments, the at least one organic substrate includes alpha-ketoglutarate, sucrose, glucose, glycerol, a monosaccharide, a disaccharide, a polysaccharide, or a combination thereof. In certain embodiments, the at least one organic product includes ethylene, an alcohol, methanol, ethanol, propanol, butanol, ethane diol, an organic acid, propionic acid, acetic acid, an aldehyde, formaldehyde, a long chain fatty acid, an n-alkane, a hydrocarbon, or a combination thereof.

In certain embodiments, the carbon source includes carbon dioxide, carbon monoxide, glycerol, glucose, fructose, sucrose, a monosaccharide, a disaccharide, a polysaccharide, glycogen, acetic acid, a fatty acid, or a combination thereof. In certain embodiments, the power source includes sunlight, a solar power source, an electrical power source, or a combination thereof. In certain embodiments, the volatile gas includes oxygen, methane, or a combination thereof. In certain embodiments, the at least one organic substrate includes alpha-ketoglutarate, sucrose, glucose, fructose, xylose, arabinose, galactose, glycerol, a monosaccharide, a disaccharide, a polysaccharide, glycogen, a fatty acid, or a combination thereof. In certain embodiments, the at least one organic product includes an alcohol, methanol, ethanol, propanol, butanol, ethane diol, an organic acid, propionic acid, acetic acid, an aldehyde, formaldehyde, a long chain fatty acid, an n-alkane, a hydrocarbon, ethane, propene, butene, ethane, propane, butane, or a combination thereof. In certain embodiments, the second bioreactor culture vessel further includes a carbon source inlet, a volatile gas outlet, a power source, or a combination thereof.

In certain embodiments, the organic substrate culture solution and the organic product culture solution are combined in one bioreactor culture vessel. In other embodiments, the organic substrate culture solution and the organic product culture are separated by a filter. In certain embodiments, the filter includes a pore size of from about 0.2 μm to about 10 μm or more.

In certain embodiments of a system herein, the at least one organic substrate includes alpha-ketoglutarate (AKG), wherein an amount of the at least one AKG forming enzyme produced by the first recombinant microorganism is greater than that produced relative to a control microorganism lacking a non-native AKG forming enzyme expressing nucleotide sequence. In such embodiments, the at least one organic product includes ethylene, wherein an amount of the at least one ethylene forming enzyme (EFE) produced by the second recombinant microorganism is greater than that produced relative to a control microorganism lacking a non-native EFE expressing nucleotide sequence. In certain embodiments, the first recombinant microorganism expresses at least one alpha-ketoglutarate permease protein (AKGP) by expressing at least one non-native AKGP forming nucleotide sequence.

In certain embodiments, the at least one AKG forming enzyme includes an isocitrate dehydrogenase (ICD) protein, a glutamate dehydrogenase (GDH) protein, or a combination thereof. In certain embodiments, the first recombinant microorganism expresses an ICD protein having an amino acid sequence at least 95% identical to SEQ ID NO: 1 or SEQ ID NO: 3 by expressing a non-native ICD protein nucleotide sequence having a nucleotide sequence at least 95% identical to SEQ ID NO: 2 or SEQ ID NO: 4, or the first recombinant microorganism expresses a GDH protein having an amino acid sequence at least 95% identical to SEQ ID NO: 5 by expressing a non-native GDH protein nucleotide sequence having a nucleotide sequence at least 95% identical to SEQ ID NO: 6, or a combination thereof. In certain embodiments, the second recombinant microorganism expresses an EFE protein having an amino acid sequence at least 95% identical to SEQ ID NO: 7 by expressing a non-native EFE protein nucleotide sequence having a nucleotide sequence at least 95% identical to SEQ ID NO: 8.

In certain embodiments, the first recombinant microorganism includes a microorganism selected from the group consisting of a photosynthetic bacteria, a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, and Chlamydomonas reinhardtii. In certain embodiments, the second recombinant microorganism includes a microorganism selected from the group consisting of Escherichia, Escherichia coli, Geobacteria, Arthrobacter paraffineus, Pseudomonas fluorescens, Pseudomonas Putida, a Pseudomonas, Pseudomonas syringae, Pseudomonas savastanoi, Serratia marcescens, Bacillus metatherium, Candida paludigena, Pichia inositovora, Torulopsis glabrata, Candida lipolytica, Yarrowia lipolytica, Saccharomyces cereviciae, Aspergillus sp., Bacillus subtilis, and Lactobacillus sp.

In certain embodiments, the first recombinant microorganism includes a delta-glgc mutant microorganism lacking expression of a glucose-1-phosphate adenylyltransferase protein. In certain embodiments, the first recombinant microorganism expresses a sucrose synthase protein having an amino acid sequence at least 95% identical to SEQ ID NO: 9 by expressing a non-native sucrose synthase protein nucleotide sequence having a nucleotide sequence at least 95% identical to SEQ ID NO: 10. In certain embodiments, the first recombinant microorganism expresses a sucrose phosphate synthase protein having an amino acid sequence at least 95% identical to SEQ ID NO: 11 by expressing a non-native sucrose phosphate synthase nucleotide sequence having a nucleotide sequence at least 95% identical to SEQ ID NO: 12.

In certain embodiments, an amount of at least one AKG forming enzyme produced by the first recombinant microorganism is from about 5% to about 200% or more greater than that produced relative to the control microorganism lacking the non-native AKG forming enzyme expressing nucleotide sequence. In certain embodiments, an amount of EFE protein produced by the second recombinant microorganism is from about 5% to about 200% or more greater than that produced relative to the control microorganism lacking the non-native EFE expressing nucleotide sequence. In certain embodiments, an amount of EFE protein produced by the second recombinant microorganism is from about 20 grams to about 100 grams per liter or more of organic product culture solution.

In certain embodiments, the second recombinant microorganism includes E. coli, and an amount of EFE protein produced by the second recombinant microorganism is from about 30% to about 80% or more of a total cellular amount of protein of the second recombinant microorganism. In certain embodiments, a production rate of ethylene produced by the second recombinant microorganism is from about 100 million pounds/year to about 1 billion pounds/year or more. In certain embodiments, a cell population concentration of the second recombinant microorganism ranges from about 10⁷ to about 10¹³ cells per milliliter, or a dry cell weight per liter of organic product culture solution of from about 100 grams to about 300 grams dry cell weight per liter.

In certain embodiments, the non-native AKG forming enzyme expressing nucleotide sequence or the non-native EFE expressing nucleotide sequence is inserted into a microbial expression vector, wherein the microbial expression vector includes a bacterial vector plasmid, a nucleotide guide of a homologous recombination system, an antibiotic-resistant system, an aid system for protein purification and detection, a CRISPR CAS system, a phage display system, or a combination thereof. In certain embodiments, the EFE expressing nucleotide sequence has a copy number in the microbial expression vector of from about 2 to about 500 or more. In certain embodiments, the microbial expression vector includes at least one microbial expression promoter. In certain embodiments, the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously inducing promoter, a psbA promoter, or a combination thereof.

Embodiments herein are directed to methods of producing an organic product. In an embodiment, the method includes providing a biomanufacturing system including: at least one bioreactor culture vessel; wherein the at least one bioreactor culture vessel contains an organic substrate culture solution; wherein the organic substrate culture solution contains a first recombinant microorganism; wherein the first recombinant microorganism has an improved organic substrate producing ability, expresses at least one organic substrate forming recombinant enzyme by expressing at least one non-native organic substrate forming enzyme nucleotide sequence, is capable of utilizing a carbon source to produce the organic substrate, and produces at least one organic substrate culture impurity, including at least one of a volatile gas, a liquid, and a solid; wherein the at least one bioreactor culture vessel contains an organic product culture solution; wherein the organic product culture solution contains a second recombinant microorganism; wherein the second recombinant microorganism has an improved organic product producing ability, expresses at least one organic product forming enzyme by expressing at least one non-native organic product forming enzyme nucleotide sequence, and is capable of utilizing the organic substrate to produce the at least one organic product; wherein the at least one bioreactor culture vessel includes a first bioreactor culture vessel including a carbon source inlet, a volatile gas outlet, and a power source; and the system includes a second bioreactor culture vessel including a fluid flow path connected to and between the first bioreactor culture vessel and the second bioreactor culture vessel, and an organic product outlet; wherein the first bioreactor culture vessel includes the organic substrate culture solution, and the second bioreactor culture vessel includes the organic product culture solution. In such embodiments, the method includes: culturing the first recombinant microorganism in the first bioreactor culture vessel under conditions sufficient to produce the amount of at least one organic substrate in the first bioreactor culture vessel; and culturing the second recombinant microorganism in the second bioreactor culture vessel under conditions sufficient to produce the amount of at least one organic product in the second bioreactor culture vessel.

In certain embodiments, the method further includes removing an amount of the at least one volatile gas from the organic substrate culture solution through the volatile gas outlet. In certain embodiments, the method further includes removing an amount of the at least one organic product from the organic product culture solution through the organic product outlet. In certain embodiments, provided the carbon source includes carbon dioxide, the method further includes feeding an amount of the carbon dioxide from a carbon dioxide source into the organic substrate culture solution through the carbon source inlet. In certain embodiments, the method further includes maintaining a pH level of the organic substrate culture solution and the organic product culture solution from about 5.0 to about 8.5. In certain embodiments, the method includes maintaining the organic substrate culture solution and the organic product culture solution at a temperature from about 25 degrees Celsius to about 70 degrees Celsius or more.

In certain embodiments, the method further includes maintaining an amount of volatile gas in the second bioreactor culture vessel of from about 10% by volume to about 1% by volume or less, based a total internal volume of the second bioreactor culture vessel.

In certain embodiments, provided the first bioreactor culture vessel or the second bioreactor culture vessel further includes a biomass collection port, the method further includes collecting an amount of biomass produced by the first recombinant microorganism or the second recombinant microorganism through the biomass collection port.

In certain embodiments of methods herein, the non-native organic substrate forming recombinant enzyme expressing nucleotide sequence or the non-native organic product expressing nucleotide sequence is inserted into a microbial expression vector, wherein the at least one microbial expression vector includes at least one microbial expression promoter. In certain embodiments, the method further includes controlling the amount of the at least one organic substrate or the amount of the at least one organic product produced by adding at least one promoter inducer to the organic substrate culture solution or the organic product culture solution. In certain embodiments, the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof; and the at least one promoter inducer includes lactose, xylose, IPTG, cold shock, heat shock, or a combination thereof.

In certain embodiments of methods herein, the at least one organic substrate includes AKG. In such embodiments, the at least one organic product includes ethylene. In certain embodiments, provided the at least one organic substrate culture impurity includes at least one volatile gas, the method further includes removing the at least one volatile gas through the volatile gas outlet. In certain embodiments, the method includes recovering an amount of ethylene produced at a rate of from about 100 million pounds/year to about 1 billion pounds/year or more. In certain embodiment, the amount of ethylene produced contains an amount of volatile gas of about 1 mole percent or less.

Embodiments herein are directed to methods of producing an organic product, wherein the method includes providing a biomanufacturing system including: at least one bioreactor culture vessel; wherein the at least one bioreactor culture vessel contains an organic substrate culture solution; wherein the organic substrate culture solution contains a first recombinant microorganism; wherein the first recombinant microorganism has an improved organic substrate producing ability, expresses at least one organic substrate forming recombinant enzyme by expressing at least one non-native organic substrate forming enzyme nucleotide sequence, is capable of utilizing a carbon source to produce the organic substrate, and produces at least one organic substrate culture impurity, including at least one of a volatile gas, a liquid, and a solid; and wherein the at least one bioreactor culture vessel contains an organic product culture solution; wherein the organic product culture solution contains a second recombinant microorganism; wherein the second recombinant microorganism has an improved organic product producing ability, expresses at least one organic product forming enzyme by expressing at least one non-native organic product forming enzyme nucleotide sequence, and is capable of utilizing the organic substrate to produce the at least one organic product. In such embodiments, the organic substrate culture solution and the organic product culture solution are combined in one bioreactor culture vessel, wherein the non-native organic substrate forming recombinant enzyme expressing nucleotide sequence is inserted into a first microbial expression vector, wherein the non-native organic product forming enzyme expressing nucleotide sequence is inserted into a second microbial expression vector, and wherein the first and second microbial expression vector each includes at least one microbial expression promoter. In such embodiments, the method includes providing a carbon source connected to the carbon source inlet; culturing the first recombinant microorganism in the first bioreactor culture vessel under conditions sufficient to produce an amount of the at least one organic substrate in the first bioreactor culture vessel; producing the amount of the at least one organic substrate by adding at least one promoter inducer to the organic substrate culture solution at a first time point; culturing the second recombinant microorganism in the second bioreactor culture vessel under conditions sufficient to produce an amount of the at least one organic product in the second bioreactor culture vessel; and producing the amount of the at least one organic product by adding at least one promoter inducer to the organic product culture solution at a second time point. In certain embodiments, the method includes lowering an amount of volatile gas in the second bioreactor culture vessel to from about 10% by volume to about 1% by volume or less, based on a total internal volume of the second bioreactor culture vessel, before the second time point.

Embodiments herein are directed to methods of producing ethylene. In certain embodiments, the method includes providing a biomanufacturing system including: a first bioreactor culture vessel including a carbon source inlet, a volatile gas outlet, and a power source; and a second bioreactor culture vessel including a fluid flow path connected to and between the first bioreactor culture vessel and the second bioreactor culture vessel, and an organic product outlet; wherein the first bioreactor culture vessel includes an organic substrate culture solution, and the second bioreactor culture vessel includes an organic product culture solution; wherein the organic substrate culture solution contains a first recombinant microorganism having an improved organic substrate producing ability, wherein the organic substrate includes AKG, wherein the first recombinant microorganism expresses at least one organic substrate forming recombinant enzyme by expressing at least one non-native organic substrate forming enzyme nucleotide sequence, wherein the organic substrate forming enzyme includes an AKG forming enzyme, wherein the first recombinant microorganism is capable of utilizing a carbon source to produce an amount of AKG, wherein the first recombinant microorganism produces at least one organic substrate culture impurity, including at least one of a volatile gas, a liquid, and a solid; wherein the volatile gas includes oxygen, wherein the organic product culture solution contains a second recombinant microorganism having an improved organic product producing ability, wherein the organic product includes ethylene, wherein the second recombinant organism expresses at least one organic product forming enzyme by expressing at least one non-native organic product forming enzyme nucleotide sequence, wherein the organic product forming enzyme includes EFE, and wherein the second recombinant organism is capable of utilizing AKG to produce an amount of ethylene. In certain embodiments, the method includes culturing the first recombinant microorganism in the first bioreactor culture vessel under conditions sufficient to produce an amount of AKG in the first bioreactor culture vessel; culturing the second recombinant microorganism in the second bioreactor culture vessel under conditions sufficient to produce an amount of EFE in the second bioreactor culture vessel; removing an amount of oxygen from the organic substrate culture solution through the volatile gas outlet; and removing an amount of EFE from the organic product culture solution through the organic product outlet.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 is an illustration depicting production of an organic substrate and an organic product by microorganisms according to embodiments herein.

FIG. 2 is an illustration of a biomanufacturing system according to embodiments herein.

FIG. 3A is a graph showing ethylene production in E. coli cultures expressing low, medium, or high copy numbers of an EFE gene, and cultured in different growth media, according to embodiments herein.

FIG. 3B is a graph showing ethylene production in E. coli cultures grown with no supplement or with different growth supplements, according to embodiments herein.

FIG. 4 is a flow chart depicting an embodiment of a method of producing an organic product herein.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one bioreactor culture vessel” means one bioreactor culture vessel, more than one bioreactor culture vessel, or any combination thereof.

Unless otherwise noted, the term “about” refers to ±10% of the non-percentage number that is described, rounded to the nearest whole integer. For example, about 20 grams, would include 18 to 22 grams. Unless otherwise noted, the term “about” refers to ±5% of a percentage number. For example, about 50% would include 45 to 55%. When the term “about” is discussed in terms of a range, then the term refers to the appropriate amount less than the lower limit and more than the upper limit. For example, from about 100 to about 300 grams dry cell weight per liter would include from 90 to 330 grams dry cell weight per liter.

Unless otherwise noted, properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any method or system of any embodiment herein.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by various methods, known to those skilled in the art. In an embodiment, sequence identity is determined by comparing the whole length of the sequences as identified herein.

Exemplary methods to determine identity are designed to give the largest match between the sequences tested. Methods to determine identity and similarity are codified in publicly available computer programs. Exemplary computer program methods to determine identity and similarity between two sequences include e.g. the BestFit, BLASTP (Protein Basic Local Alignment Search Tool), BLASTN (Nucleotide Basic Local Alignment Search Tool), and FASTA (Altschul, S. F. et al., J. Mol. Biol. 215:403-410 (1990), publicly available from NCBI and other sources (BLAST® Manual, Altschul, S., et al., NCBI NLM NIH Bethesda, Md. 20894). A most exemplary algorithm used is EMBOSS (European Molecular Biology Open Software Suite). Exemplary parameters for amino acid sequences comparison using EMBOSS are gap open 10.0, gap extend 0.5, Blosum matrix. Exemplary parameters for nucleic acid sequences comparison using EMBOSS are gap open 10.0, gap extend 0.5, DNA full matrix (DNA identity matrix). In embodiments, it is possible to compare the DNA/protein sequences among different species to determine the homology of sequences using online data such as Gene bank, KEG, BLAST and Ensemble.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gin; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Unless otherwise noted, the term “adapted” or “codon adapted” refers to “codon optimization” of polynucleotides as disclosed herein, the sequence of which may be native or non-native, or may be adapted for expression in other microorganisms. Codon optimization adapts the codon usage for an encoded polypeptide towards the codon bias of the organism in which the polypeptide is to be expressed. Codon optimization generally helps to increase the production level of the encoded polypeptide in the host cell.

Carbon dioxide emissions resulting from the use of fossil fuels continue to rise on a global scale. Reduction of atmospheric carbon dioxide levels is a key to mitigating or reversing climate change. Carbon capture and storage (CCS) is a prominent technology for removal of industrial carbon dioxide from the atmosphere; it has been estimated that over 20 trillion tons of carbon dioxide captured from refining and other industrial processes can be transported and stored in various types of subterranean environments or storage tanks. Although CCS is a cost effective and affordable way to reduce carbon dioxide emissions compared to other currently available methods, the problem remains that the carbon dioxide is merely being stored underground until it escapes. Therefore, CCS methods do not provide a sustainable solution to reduce excess carbon dioxide in the atmosphere. Also, there is little financial incentive for industries to pump carbon dioxide into subterranean environments, unless they are forced to by environmental regulations, or they are paid to do it as part of their business model. Arguably, global warming is a crisis because it is more lucrative to produce carbon dioxide than to dispose of carbon dioxide.

There remains a need to remove excess carbon dioxide from the atmosphere in more efficient and sustainable ways. There remains a need for technologies that can harness the over-abundance of carbon dioxide to make useful products, and for other applications that are beneficial to industry and the environment.

The challenges of the limited supply of petroleum, and the harmful effects of petroleum operations on the environment, have prompted a growing emphasis on maximizing output from existing resources, and in developing renewable sources of fuels and chemicals that can minimize environmental impacts. Technologies that convert biomass and captured carbon dioxide into new products, such as biofuels, can help to reduce oil imports and carbon dioxide emissions. There is a growing interest in producing value-added fuels and other useful organic products from organic waste using biological processes.

Among such valuable organic products, ethylene is the most widely produced organic compound in the world, useful in a broad spectrum of industries including plastics, solvents, and textiles. Since ethylene is a potentially renewable feedstock, there has been a great deal of interest in developing technologies to produce ethylene from renewable sources, such as carbon dioxide and biomass. Ethylene is currently produced by steam cracking fossil fuels or dehydrogenating ethane. With millions of metric tons of ethylene being produced each year, however, more than enough carbon dioxide is produced by such processes to greatly contribute to the global carbon footprint. Producing ethylene through renewable methods would accordingly help to meet the huge demand from the energy and chemical industries, while also helping to protect the environment.

Conventional methods have been developed to produce bio-ethylene using ethanol derived from corn or sugar cane. However, the production of bio-ethylene from biomass (e.g., corn and sugar cane) is a time-consuming and cost-ineffective process, requiring land, transportation, and digestion of biomass. For example, there are massive inefficiencies associated with the growing and transportation of corn and sugar cane, which by itself causes CO₂ emissions. A variety of microbes, including bacteria and fungi, naturally produce ethylene in small amounts. Such microbes make use of an ethylene-forming enzyme (EFE). A type of ethylene pathway, such as is found in Pseudomonas syringae and Penicillium digitatum, uses alpha-ketoglutarate (AKG) and arginine as substrates in a reaction catalyzed by an ethylene-forming enzyme. Ethylene-forming enzymes provide a promising target, because expression of a single gene can be sufficient for ethylene production. Techniques making use of heterologous expression of an EFE have been demonstrated in several microbial species, where the microbial hosts have been able to utilize a variety of carbon sources in the Calvin cycle, including lignocellulose and carbon dioxide. Plus, recent developments in cost-effective high throughput genetic sequencing technologies have led to an increased understanding of microbial gene expression. However, the currently available technologies do not produce industrially relevant quantities of ethylene through microbial activity. As a result, the number and volume of bioreactors required for production of sufficient quantities of ethylene can be prohibitively high.

Another challenge that can arise in microbial organic product production processes is that by-products of the microbial metabolism can be produced as impurities in the bioreactor culture along with the organic product. A mixture of organic product and by-product impurities can add more complications to the downstream purification process, and additional costs. Culture impurities can include volatile gases such as oxygen, which may be co-produced with volatile organic products. For example, oxygen is typically produced by photosynthetic microorganisms. The concentration of oxygen in the bioreactor off-gas can possibly be high enough to present a safety risk, or exceed the maximum amounts allowed by regulations. Yet another challenge is how to handle processing of another by-product: the biomass that is produced as a result of the growth of microbial cultures.

There remains a need for improvements in microbial production that can produce organic products, including ethylene, at a commercial scale with greater efficiency and lower costs. There remains a need for improvements in microbial production that can reduce or eliminate the co-production of organic products and culture impurities, including volatile gases, safely while complying with governmental regulations. There remains a need for improvements in the production and handling of biomass in microbial organic product production. There remains a need for methods to use carbon dioxide feedstocks to produce organic products, such as bio-ethylene, that are useful for industrial and other applications.

Embodiments of the present disclosure can provide a benefit of removing carbon dioxide from the environment, along with the benefit of producing valuable organic products capable of being sold commercially. Embodiments of the present disclosure can thus provide a renewable alternative to conventional carbon dioxide storage, by using recombinant microbial technology to convert the carbon dioxide into one or more useful organic compounds. One benefit of the embodiments of the present disclosure is that the systems and methods can make it economically profitable for an oil or natural gas company to remove carbon dioxide from the environment. An oil company, or a contractor thereof, could instead of pumping carbon dioxide into a subterranean environment or leaving the sequestered carbon dioxide underground, use the carbon dioxide as a carbon source for a culture of recombinant microorganisms to convert the carbon dioxide to useful organic products in a cost-effective way. Also, much of the carbon dioxide generated by transportation can be avoided, because the methods can be practiced on-site, or would be expected to consume more carbon dioxide than they produce.

The most effective methods for protecting the environment are those methods that people actually use. The more profitable those methods are; the more likely people are to use them. One of the benefits of the methods disclosed herein is the cost-effectiveness of using a bioreactor system. Embodiments of the present disclosure can provide a benefit of engineering a photosynthetic organic substrate producing microorganism, by adapting the relevant metabolic signaling pathways to produce the organic substrate on an industrial scale. Embodiments of the present disclosure can provide a benefit of engineering an organic product producing microorganism that can utilize the organic substrate produced by the photosynthetic microorganism to produce the organic product, by adapting the relevant metabolic signaling pathways to produce the organic product on an industrial scale. Such embodiments can provide the benefits of increased efficiency of organic product production and lower costs. Such embodiments can make it profitable to remove carbon dioxide from the atmosphere, and to passively generate valuable organic compounds while the microbes do the work—on a scale previously unimaginable.

Embodiments of the present disclosure can also provide a benefit of separating the production of one or more culture impurities from the production of organic products, thus reducing or eliminating the co-production of the organic products and culture impurities. Such embodiments can provide benefits of safer organic product production, as well as reducing the complications and costs of downstream purification. Embodiments herein can also provide benefits of minimizing biomass production, while facilitating the use of the biomass that is produced for beneficial applications.

What would happen to the global warming crisis if it became more profitable, or just as profitable, to convert carbon dioxide into valuable organic compounds as it did to generate the carbon dioxide in the first place? The presently disclosed methods might transform energy producers from global warming companies to global cooling companies.

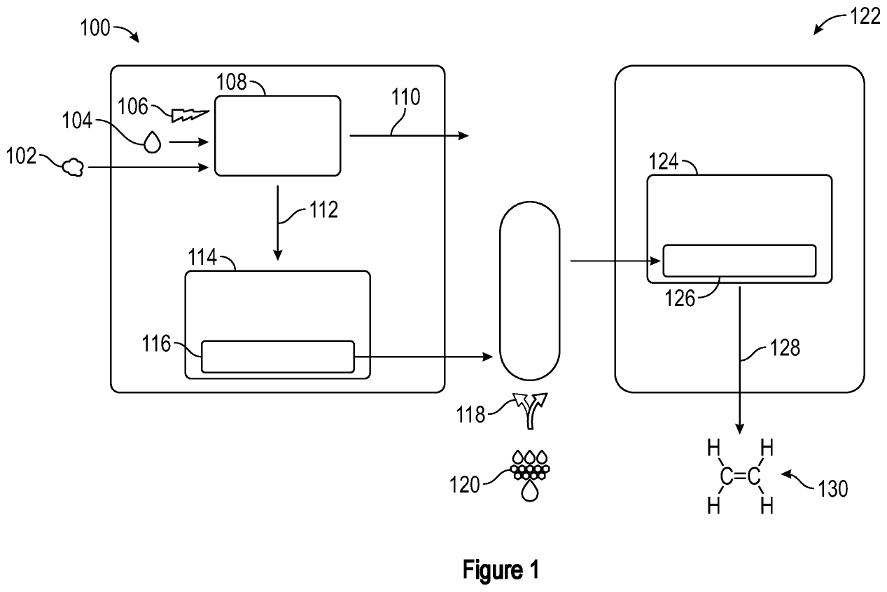

The present disclosure relates to biomanufacturing systems for producing an organic product. In certain embodiments, such a system includes an organic substrate culture solution containing a first recombinant microorganism having an improved substrate producing ability, and an organic product culture solution containing a second recombinant microorganism having an improved organic product producing ability. As a general overview of organic substrate production and organic product production by the organic substrate culture solution and the organic product culture solution, respectively, referring to FIG. 1, organic substrate culture solution 100 captures carbon dioxide 102 together with water 104 and light 106 in photosynthetic light reactions 108, producing culture impurity oxygen 110 and energy substrates 112; enzymatic pathways 114 utilize energy substrates 112 to produce organic substrate alpha-ketoglutarate 116. Organic substrate alpha-ketoglutarate 116 enters organic product culture solution 122 through production by promoter inducer control at time points 118, or by filter or flow path separation 120. Enzymatic pathways 124 utilize organic product culture solution alpha-ketoglutarate 126 and ethylene forming enzyme 128 to produce organic product ethylene 130.

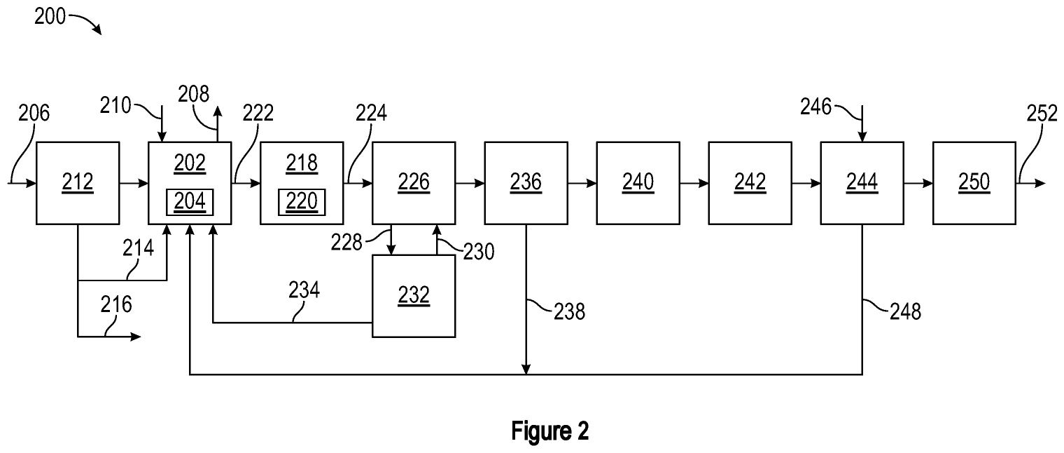

As a general overview of a biomanufacturing system disclosed herein, referring to FIG. 2, system 200 includes a first bioreactor culture vessel 202 containing organic substrate culture solution 204, carbon source inlet 206, volatile gas outlet 208, power and/or light source 210, compressor/condenser 212 including water outlet 214 connected to first bioreactor culture vessel 202 and water outlet 216; second bioreactor culture vessel 218 containing organic product culture solution 220 and including fluid flow path 222 connected to and between first bioreactor culture vessel 202 and second bioreactor culture vessel 218, and organic product outlet 224; amine stripper 226 including rich amine outlet 228 and lean amine inlet 230 connected to amine scrubber 232; carbon dioxide outlet 234 connected to and between amine scrubber 232 and first bioreactor culture vessel 202; compressor/condenser 236 including water outlet 238; catalytic converter 240; caustic tower 242; dryer 244 including regeneration gas inlet 246 and water outlet 248; compressor/pump 250; and organic product outlet 252.

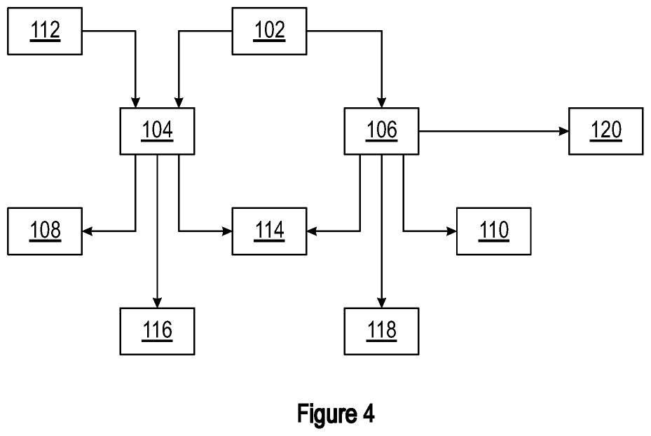

The present disclosure related to methods for producing an organic product. As a general overview of a method disclosed herein, referring to FIG. 4, the method includes providing a biomanufacturing system 102 according to embodiments herein; culturing a first recombinant microorganism in a first bioreactor culture vessel containing an organic substrate culture solution, under conditions sufficient to produce an amount of at least one organic substrate in the first bioreactor culture vessel 104; culturing a second recombinant microorganism in a second bioreactor culture vessel containing an organic product culture solution, under conditions sufficient to produce an amount of at least one organic product in the second bioreactor culture vessel 106; removing an amount of at least one volatile gas from the organic substrate culture 108; removing an amount of at least one organic product from the organic product culture solution 110; feeding an amount of carbon dioxide from a carbon dioxide source into the organic substrate culture solution 112; maintaining a pH level of the organic substrate culture solution and the organic product culture solution from about 5.0 to about 8.5 114; collecting an amount of biomass produced by the first recombinant microorganism from the first bioreactor culture vessel 116, collecting an amount of biomass produced by the second recombinant microorganism from the second bioreactor culture vessel 118; and maintaining an amount of volatile gas in the second bioreactor culture vessel of from about 10% by volume to about 1% by volume or less, based on a total internal volume of the second bioreactor culture vessel 120.

### Embodiments of Biomanufacturing Systems

Embodiments herein are directed to biomanufacturing systems for producing an organic product. In various embodiments, the system includes at least one bioreactor culture vessel. In various embodiments, the at least one bioreactor culture vessel contains an organic substrate culture solution, wherein the organic substrate culture solution contains a first recombinant microorganism. In various embodiments, the first recombinant microorganism has an improved organic substrate producing ability, expresses at least one organic substrate forming recombinant enzyme by expressing at least one non-native organic substrate forming enzyme nucleotide sequence, is capable of utilizing a carbon source to produce the organic substrate, and produces at least one organic substrate culture impurity, including at least one of a volatile gas, a liquid, and a solid byproduct, or other undesirable product. An example of an impurity could be oxygen or methane, or a combination thereof, in a gas or liquid state. In various embodiments, the at least one bioreactor culture vessel contains an organic product culture solution, wherein the organic product culture solution contains a second recombinant microorganism. In various embodiments, the second recombinant microorganism has an improved organic product producing ability, expresses at least one organic product forming enzyme by expressing at least one non-native organic product forming enzyme nucleotide sequence, and is capable of utilizing the organic substrate to produce the at least one organic product. In various embodiment, the at least one organic product can include an alkene, an alkane, a polyene, an alcohol, a volatile organic compound, or a combination thereof.

In certain embodiments, the at least one bioreactor culture vessel includes a first bioreactor culture vessel including a carbon source inlet, a volatile gas outlet, and a power source; and a second bioreactor culture vessel including a fluid flow path connected to and between the first bioreactor culture vessel and the second bioreactor culture vessel, and an organic product outlet. In such embodiments, the first bioreactor culture vessel includes the organic substrate culture solution, and the second bioreactor culture vessel includes the organic product culture solution. Such embodiments can provide a benefit of separating the production of the at least one organic substrate culture impurity by the first recombinant microorganism, from the production of the at least organic product by the second recombinant organism. In such embodiments, the organic substrate and other nutrients can be provided to the second recombinant microorganism through the fluid flow path connected to and between the first bioreactor culture vessel and the second bioreactor culture vessel, to provide for growth of the second recombinant microorganism and production of the organic product. In such embodiments, provided the at least one organic substrate culture impurity includes a volatile gas, the volatile gas can be removed from the organic substrate culture solution through the volatile gas outlet. In certain embodiments, the volatile gas includes oxygen, methane, or a combination thereof. Such embodiments can provide benefits of improved control of the growth rates of the first and second recombinant microorganisms, improved rates of carbon capture, and improved organic product yield.

In such embodiments, a carbon source can be provided through the carbon source inlet, as a nutrient to support the growth of the first recombinant microorganism and production of the at least one organic substrate. In certain embodiments, the carbon source includes carbon dioxide, carbon monoxide, a vegetable oil, glycerol, glucose, sucrose, a monosaccharide, a disaccharide, a polysaccharide, or a combination thereof. Such embodiments can provide a benefit of utilizing industrial carbon dioxide toward the production of an organic product. In certain embodiments, the at least one organic substrate produced by the first recombinant microorganism includes alpha-ketoglutarate (AKG), sucrose, glucose, glycerol, a monosaccharide, a disaccharide, a polysaccharide, or a combination thereof. Such embodiments can provide a benefit of providing a non-volatile organic substrate produced in the first bioreactor culture vessel that can be utilized for production of the organic product in the second bioreactor culture vessel. In certain embodiments, the second bioreactor culture vessel further includes a carbon source inlet, a volatile gas outlet, a power source, or a combination thereof.

In certain embodiments, the at least one organic product produced by the second recombinant microorganism includes ethylene, an alcohol, methanol, ethanol, propanol, butanol, ethane diol, an organic acid, propionic acid, acetic acid, an aldehyde, formaldehyde, a long chain fatty acid, an n-alkane, a hydrocarbon, or a combination thereof. In certain embodiments, the at least one organic substrate includes AKG, and the at least one organic product includes ethylene.

In certain embodiments, the first bioreactor culture vessel or the second bioreactor culture vessel further includes a biomass collection port. In such embodiments, a biomass collection port can provide a benefit of enabling the collection of biomass from the first bioreactor culture, the second bioreactor culture, or a combination thereof, for disposal or processing of the biomass for use in fertilizer, feedstock, biofuel, or other applications.

In certain embodiments, the power source includes sunlight, a solar power source, an electrical power source, or a combination thereof. In certain embodiments, the system further includes a carbonation unit, an amine stripper, an amine scrubber, a catalytic converter, a condenser, a compressor, a caustic tower, a dryer, or combinations thereof.

In certain embodiments, the organic substrate culture solution and the organic product culture solution are combined in one bioreactor culture vessel. In some embodiments, the organic substrate culture solution and the organic product culture are separated by a filter. In such embodiments, the filter can include a pore size of from about 0.2 μm to about 10 μm or more. In certain embodiments, the filter includes a pore size of from about 1 μm to about 8 μm or more. In certain embodiments, the filter includes a pore size of from about 3 μm to about 5 μm or more. Such embodiments can provide a benefit of separating the production of the at least one organic substrate culture impurity by the first recombinant microorganism, from the production of the at least one organic product by the second recombinant organism, by allowing the flow of the at least one organic substrate and other nutrients from the organic substrate culture solution to the organic product culture solution through the filter, while not allowing or reducing the flow of the at least one organic substrate culture impurity through the filter. In certain embodiments, the organic substrate culture solution and the organic product culture solution contain suspended organic particles that are separated by a filter. In certain embodiments, the suspended organic particles can be separated from the organic substrate culture solution or the organic product culture solution with the use of a centrifuge.

In other embodiments wherein the organic substrate culture solution and the organic product culture solution are combined in one bioreactor culture vessel, the at least one non-native organic substrate forming enzyme nucleotide sequence expressed by the first recombinant microorganism, and the at least one non-native organic product forming enzyme nucleotide sequence expressed by the second recombinant microorganism, are inserted into a first microbial expression vector and a second microbial expression vector, respectively. The first and second microbial expression vectors each include at least one microbial expression promoter. Such embodiments can provide a benefit of separating the production of the at least one organic substrate culture impurity by the first recombinant microorganism, from the production of the at least one organic product by the second recombinant microorganism, by producing the at least one organic substrate and the at least one organic product at different time points, by adding at least one promoter inducer to the organic substrate culture solution or to the organic product culture solution at different time points.

### Embodiments of Recombinant Microorganisms

In certain embodiments of a system herein, the organic substrate includes alpha-ketoglutarate (AKG), wherein an amount of the at least one AKG forming enzyme produced by the first recombinant microorganism is greater than that produced relative to a control microorganism lacking a non-native AKG forming enzyme expressing nucleotide sequence. In such embodiments, the at least one organic product includes ethylene, wherein an amount of the at least one ethylene forming enzyme (EFE) produced by the second recombinant microorganism is greater than that produced relative to a control microorganism lacking a non-native EFE expressing nucleotide sequence. In certain embodiments, the first recombinant microorganism expresses at least one alpha-ketoglutarate permease protein (AKGP) by expressing at least one non-native AKGP forming nucleotide sequence. Such embodiments can provide a benefit of increasing yield of the organic substrate and the at least one organic product, thus reducing the number and volume of bioreactors required to produce ethylene on a commercial scale.

In certain embodiments, the at least one AKG forming enzyme includes an isocitrate dehydrogenase (ICD) protein, a glutamate dehydrogenase (GDH) protein, or a combination thereof. In certain embodiments, the first recombinant microorganism expresses an ICD protein having an amino acid sequence at least 95% identical to SEQ ID NO: 1 by expressing a non-native ICD protein nucleotide sequence having a nucleotide sequence at least 95% identical to SEQ ID NO: 2. In an embodiment, the ICD amino acid sequence has an amino acid sequence at least 80% or at least 90% identical to SEQ ID NO: 1. In an embodiment, the ICD amino acid sequence has an amino acid sequence at least 98% identical to SEQ ID NO: 1. In an embodiment, the ICD nucleotide sequence has a nucleotide sequence at least 80% or at least 90% identical to SEQ ID NO: 2. In an embodiment, the ICD nucleotide sequence has a nucleotide sequence at least 98% identical to SEQ ID NO: 2. In certain embodiments, the first recombinant microorganism expresses an ICD protein having an amino acid sequence at least 95% identical to SEQ ID NO: 3 by expressing a non-native ICD protein nucleotide sequence having a nucleotide sequence at least 95% identical to SEQ ID NO: 4. In an embodiment, the ICD amino acid sequence has an amino acid sequence at least 80% or at least 90% identical to SEQ ID NO: 3. In an embodiment, the ICD amino acid sequence has an amino acid sequence at least 98% identical to SEQ ID NO: 3. In an embodiment, the ICD nucleotide sequence has a nucleotide sequence at least 80% or at least 90% identical to SEQ ID NO: 4. In an embodiment, the ICD nucleotide sequence has a nucleotide sequence at least 98% identical to SEQ ID NO: 4.

In certain embodiments, the first recombinant microorganism expresses a GDH protein having an amino acid sequence at least 95% identical to SEQ ID NO: 5 by expressing a non-native GDH protein nucleotide sequence having a nucleotide sequence at least 95% identical to SEQ ID NO: 6. In an embodiment, the first recombinant microorganism expresses a GDH protein having an amino acid sequence at least 80% or at least 90% identical to SEQ ID NO: 5. In an embodiment, the first recombinant microorganism expresses a GDH protein having an amino acid sequence at least 98% identical to SEQ ID NO: 5. In an embodiment, the first recombinant microorganism expresses a GDH nucleotide sequence having a nucleotide sequence at least 80% or at least 90% identical to SEQ ID NO: 6. In an embodiment, the first recombinant microorganism expresses a GDH nucleotide sequence having a nucleotide sequence at least 98% identical to SEQ ID NO: 6.

In certain embodiments, the first recombinant microorganism expresses an ICD protein having an amino acid sequence at least 95% identical to SEQ ID NO: 1 or SEQ ID NO: 3 by expressing a non-native ICD protein nucleotide sequence having a nucleotide sequence at least 95% identical to SEQ ID NO: 2 or SEQ ID NO: 4, and a GDH protein having an amino acid sequence at least 95% identical to SEQ ID NO: 5 by expressing a non-native GDH protein nucleotide sequence having a nucleotide sequence at least 95% identical to SEQ ID NO: 6.

In certain embodiments, the second recombinant microorganism expresses an EFE protein having an amino acid sequence at least 95% identical to SEQ ID NO: 7 by expressing a non-native EFE protein nucleotide sequence having a nucleotide sequence at least 95% identical to SEQ ID NO: 8. In an embodiment, the second recombinant microorganism expresses an EFE protein having an amino acid sequence at least 80% or at least 90% identical to SEQ ID NO: 7. In an embodiment, the second recombinant microorganism expresses an EFE protein having an amino acid sequence at least 98% identical to SEQ ID NO: 7. In an embodiment, the second recombinant microorganism expresses a non-native EFE protein nucleotide sequence having a nucleotide sequence at least 80% or at least 90% identical to SEQ ID NO: 8. In an embodiment, the second recombinant microorganism expresses a non-native EFE protein nucleotide sequence having a nucleotide sequence at least 98% identical to SEQ ID NO: 8.

In certain embodiments, the first recombinant microorganism includes a microorganism selected from the group consisting of a photosynthetic bacteria, a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii. In certain embodiments, the second recombinant microorganism includes a microorganism selected from the group consisting of Escherichia, Escherichia coli, Geobacteria, Arthrobacter paraffineus, Pseudomonas fluorescens, Pseudomonas Putida, a Pseudomonas, Pseudomonas syringae, Pseudomonas savastanoi, Serratia marcescens, Bacillus metatherium, Candida paludigena, Pichia inositovora, Torulopsis glabrata, Candida lipolytica, Yarrowia lipolytica, Saccharomyces cereviciae, Aspergillus sp., Bacillus subtilis, Lactobacillus sp.

In certain embodiments, the first recombinant microorganism includes a delta-glgc (Δglgc) mutant microorganism lacking expression of a glucose-1-phosphate adenylyltransferase protein. In such embodiments, the first recombinant microorganism will shift its metabolic pathway from glycogen production in favor of production of more keto acids, including AKG. In certain embodiments, the first recombinant microorganism including a Δglgc mutation will produce and excrete increased levels of AKG. Such embodiments can provide a benefit of increasing ethylene production by the second recombinant microorganism by increasing the amount of organic substrate.

In certain embodiments, the first recombinant microorganism expresses a sucrose synthase protein having an amino acid sequence at least 95% identical to SEQ ID NO: 9 by expressing a non-native sucrose synthase protein nucleotide sequence having a nucleotide sequence at least 95% identical to SEQ ID NO: 10. In an embodiment, the first recombinant microorganism expresses a sucrose synthase protein having an amino acid sequence at least 80% or at least 90% identical to SEQ ID NO: 9. In an embodiment, the first recombinant microorganism expresses a sucrose synthase protein having an amino acid sequence at least 98% identical to SEQ ID NO: 9. In an embodiment, the first recombinant microorganism expresses a non-native sucrose synthase protein nucleotide sequence having a nucleotide sequence at least 80% or at least 90% identical to SEQ ID NO: 10. In an embodiment, the first recombinant microorganism expresses a non-native sucrose synthase protein nucleotide sequence having a nucleotide sequence at least 98% identical to SEQ ID NO: 10. Such embodiments can provide a benefit of increased sucrose production as a carbon source for growth of the first recombinant microorganism, as well as a carbon source for growth of the second recombinant microorganism.

In certain embodiments, the first recombinant microorganism expresses a sucrose phosphate synthase protein having an amino acid sequence at least 95% identical to SEQ ID NO: 11 by expressing a sucrose phosphate synthase nucleotide sequence having a nucleotide sequence at least 95% identical to SEQ ID NO: 12. In an embodiment, the first recombinant microorganism expresses a sucrose phosphate synthase protein having an amino acid sequence at least 80% or at least 90% identical to SEQ ID NO: 11. In an embodiment, the first recombinant microorganism expresses a sucrose phosphate synthase protein having an amino acid sequence at least 98% identical to SEQ ID NO: 11. In an embodiment, the first recombinant microorganism expresses a sucrose phosphate protein nucleotide sequence having a nucleotide sequence at least 80% or at least 90% identical to SEQ ID NO: 12. In an embodiment, the first recombinant microorganism expresses a sucrose phosphate protein nucleotide sequence having a nucleotide sequence at least 98% identical to SEQ ID NO: 12. Such embodiments can provide a benefit of sucrose production as an additional carbon source for growth of the first recombinant microorganism, as well as an additional carbon source for growth of the second recombinant microorganism.

In various embodiments, an amount of at least one AKG forming enzyme produced by the first recombinant microorganism is greater than that produced relative to a control microorganism lacking the non-native AKG forming enzyme expressing nucleotide sequence. In certain embodiments, an amount of at least one AKG forming enzyme produced by the first recombinant microorganism is from about 5% to about 200% or more greater than that produced relative to the control microorganism lacking the non-native AKG forming enzyme expressing nucleotide sequence. In some embodiments, an amount of at least one AKG forming enzyme produced by the first recombinant microorganism is from about 50% to about 150% or more greater than that produced relative to the control microorganism lacking the non-native AKG forming enzyme expressing nucleotide sequence. In certain embodiments, an amount of at least one AKG forming enzyme produced by the first recombinant microorganism is from about 75% to about 100% or more greater than that produced relative to the control microorganism lacking the non-native AKG forming enzyme expressing nucleotide sequence.

In various embodiments, an amount of EFE protein produced by the second recombinant microorganism is greater than that produced relative to a control microorganism lacking the non-native EFE expressing nucleotide sequence. In certain embodiments, an amount of EFE protein produced by the second recombinant microorganism is from about 5% to about 200% or more greater than that produced relative to the control microorganism lacking the non-native EFE expressing nucleotide sequence. In certain embodiments, an amount of EFE protein produced by the second recombinant microorganism is from about 50% to about 150% or more greater than that produced relative to the control microorganism lacking the non-native EFE expressing nucleotide sequence. In certain embodiments, an amount of EFE protein produced by the second recombinant microorganism is from about 75% to about 100% or more greater than that produced relative to the control microorganism lacking the non-native EFE expressing nucleotide sequence.

In certain embodiments, an amount of EFE protein produced by the second recombinant microorganism is from about 20 grams to about 100 grams per liter or more of organic product culture solution. In some embodiments, an amount of EFE protein produced by the second recombinant microorganism is from about 35 grams to about 85 grams per liter or more of organic product culture solution. In some embodiments, an amount of EFE protein produced by the second recombinant microorganism is from about 50 grams to about 70 grams per liter or more of organic product culture solution.

In certain embodiments, the second recombinant microorganism includes E. coli. Such embodiments can provide benefits of a fast growth rate of the second recombinant microorganism, and extensive tools available for genetic modifications that can increase organic product yield. In certain embodiments, an amount of EFE protein produced by the second recombinant microorganism is about 30% to about 80% or more of a total cellular amount of protein of the second recombinant microorganism. In some embodiments, an amount of EFE protein produced by the second recombinant microorganism is about 40% to about 70% or more of a total cellular amount of protein of the second recombinant microorganism. In some embodiments, an amount of EFE protein produced by the second recombinant microorganism is about 50% to about 60% or more of a total cellular amount of protein of the second recombinant microorganism.

In certain embodiments, a production rate of ethylene produced by the second recombinant microorganism is from about 100 million pounds/year to about 1 billion pounds/year or more. In some embodiments, a production rate of ethylene produced by the second recombinant microorganism is from about 200 million pounds/year to about 800 million pounds/year or more. In some embodiments, a production rate of ethylene produced by the second recombinant microorganism is from about 400 million pounds/year to about 600 million pounds/year or more. In certain embodiments, a production rate of ethylene produced by the second recombinant microorganism is from about 0.13 to about 8.3 pounds per gallon of bioreactor culture per month. At scale, about 0.13 pounds/gallon-bioreactor/month productivity can be translated to an industrial plant with up to 642 commercial scale (1,000,000 gallon) bioreactors, for an annual production of up to about 1 billion pounds or more of ethylene. If productivity is increased to about 8.3 pounds/gallon-bioreactor/month, 10 commercial scale (1,000,000 gallon) bioreactors would be sufficient for an industrial plant for about 1 billion pounds/year or more of ethylene production.

In certain embodiments, a cell population concentration of the second recombinant microorganism ranges from about 10⁷ to about 10¹³ cells per milliliter. In some embodiments, a concentration of the second recombinant microorganism ranges from about 10⁸ to about 10¹² cells per milliliter. In some embodiments, a concentration of the second recombinant microorganism ranges from about 10⁹ to about 10¹¹ cells per milliliter. In certain embodiments, a concentration of the second recombinant microorganism ranges from a dry cell weight per liter of organic product culture solution of from about 100 grams to about 300 grams dry cell weight per liter, or from about 0.2 grams to about 100 grams dry cell weight per liter. In certain embodiments, a concentration of the second recombinant microorganism ranges from a dry cell weight per liter of organic product culture solution of from about 125 grams to about 275 grams dry cell weight per liter. In certain embodiments, a concentration of the second recombinant microorganism ranges from a dry cell weight per liter of organic product culture solution of from about 150 grams to about 250 grams dry cell weight per liter.

In certain embodiments, the non-native AKG forming enzyme expressing nucleotide sequence or the non-native EFE expressing nucleotide sequence is inserted into a microbial expression vector, wherein the microbial expression vector includes a bacterial vector plasmid, a nucleotide guide of a homologous recombination system, an antibiotic-resistant system, an aid system for protein purification and detection, a CRISPR CAS system, a phage display system, or a combination thereof. In certain embodiments, the EFE expressing nucleotide sequence has a copy number in the microbial expression vector of from about 2 to about 500 or more. In some embodiments, the EFE expressing nucleotide sequence has a copy number in the microbial expression vector of from about 10 to about 300. In some embodiments, the EFE expressing nucleotide sequence has a copy number in the microbial expression vector of from about 50 to about 100. Such embodiments can provide a benefit of increased ethylene yield, thus reducing the volume and cost of ethylene production on a commercial scale.

In certain embodiments, the microbial expression vector includes at least one microbial expression promoter. In certain embodiments, the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof.

### Embodiments of Methods of Producing an Organic Product

Embodiments herein are directed to methods of producing an organic product. In an embodiment, the method includes providing a biomanufacturing system including: at least one bioreactor culture vessel; wherein the at least one bioreactor culture vessel contains an organic substrate culture solution; wherein the organic substrate culture solution contains a first recombinant microorganism; wherein the first recombinant microorganism has an improved organic substrate producing ability, expresses at least one organic substrate forming recombinant enzyme by expressing at least one non-native organic substrate forming enzyme nucleotide sequence, is capable of utilizing a carbon source to produce the organic substrate, and produces at least one organic substrate culture impurity, including at least one of a volatile gas, a liquid, and a solid; wherein the at least one bioreactor culture vessel contains an organic product culture solution; wherein the organic product culture solution contains a second recombinant microorganism; wherein the second recombinant microorganism has an improved organic product producing ability, expresses at least one organic product forming enzyme by expressing at least one non-native organic product forming enzyme nucleotide sequence, and is capable of utilizing the organic substrate to produce the at least one organic product; wherein the at least one bioreactor culture vessel includes a first bioreactor culture vessel including a carbon source inlet, a volatile gas outlet, and a power source; and the system includes a second bioreactor culture vessel including a fluid flow path connected to and between the first bioreactor culture vessel and the second bioreactor culture vessel, and an organic product outlet; wherein the first bioreactor culture vessel includes the organic substrate culture solution, and the second bioreactor culture vessel includes the organic product culture solution.

In such embodiments, the method includes: culturing the first recombinant microorganism in the first bioreactor culture vessel under conditions sufficient to produce the amount of at least one organic substrate in the first bioreactor culture vessel; and culturing the second recombinant microorganism in the second bioreactor culture vessel under conditions sufficient to produce the amount of at least one organic product in the second bioreactor culture vessel. Such embodiments can provide a benefit of producing of the at least one organic substrate culture impurity by the first recombinant microorganism separately from producing the at least organic product by the second recombinant organism. In such embodiments, the organic substrate and other nutrients can be provided to the second recombinant microorganism through the fluid flow path connected to and between the first bioreactor culture vessel and the second bioreactor culture vessel, to provide for growth of the second recombinant microorganism and production of the organic product.

In certain embodiments, the method further includes removing an amount of the at least one volatile gas from the organic substrate culture solution through the volatile gas outlet. In certain embodiments, the method further includes removing an amount of the at least one organic product from the organic product culture solution through the organic product outlet. In certain embodiments, provided the carbon source includes carbon dioxide, the method further includes feeding an amount of the carbon dioxide from a carbon dioxide source into the organic substrate culture solution through the carbon source inlet. Such embodiments can provide benefits of improved control of the growth rates of the first and second recombinant microorganisms, improved rates of carbon capture, and improved organic product yield.

In certain embodiments, the method further includes maintaining a pH level of the organic substrate culture solution and the organic product culture solution from about 5.0 to about 8.5. In certain embodiments, the method further includes maintaining a pH level of the organic substrate culture solution and the organic product culture solution from about 5.5 to about 8.0. In certain embodiments, the method further includes maintaining a pH level of the organic substrate culture solution and the organic product culture solution from about 6.0 to about 7.0.

In certain embodiments, the method further includes maintaining the organic substrate culture solution and the organic product culture solution at a temperature of from about 25 degrees Celsius to about 70 degrees Celsius or more. In certain embodiments, the method further includes maintaining the organic substrate culture solution and the organic product culture solution at a temperature of from about 35 degrees Celsius to about 60 degrees Celsius. In certain embodiments, the method further includes maintaining the organic substrate culture solution and the organic product culture solution at a temperature of from about 40 degrees Celsius to about 50 degrees Celsius. In certain embodiments, the method further includes maintaining an amount of volatile gas in the second bioreactor culture vessel of from about 10% by volume to about 1% by volume or less, based on a total internal volume of the second bioreactor culture vessel. In certain embodiments, the method further includes maintaining an amount of volatile gas in the second bioreactor culture vessel of from about 5% by volume to about 0.5% by volume or less, based on a total internal volume of the second bioreactor culture vessel. In certain embodiments, the method further includes maintaining an amount of volatile gas in the second bioreactor culture vessel of from about 1% by volume to about 0.1% or less, based on a total internal volume of the second bioreactor culture vessel. Such embodiments can provide a benefit of production of the at least one organic product containing safe levels of volatile gas. Such embodiments can provide a benefit of production of the at least one organic product containing volatile gas levels within regulatory limits.

In certain embodiments, provided the first bioreactor culture vessel or the second bioreactor culture vessel further includes a biomass collection port, the method further includes collecting an amount of biomass produced by the first recombinant microorganism or the second recombinant microorganism through the biomass collection port. Such embodiments can provide a benefit of collecting biomass for disposal or processing of the biomass for use in fertilizer, feedstock, biofuel, or other applications.

In certain embodiments of methods herein, the non-native organic substrate forming recombinant enzyme expressing nucleotide sequence or the non-native organic product expressing nucleotide sequence is inserted into a microbial expression vector, wherein the at least one microbial expression vector includes at least one microbial expression promoter. In certain embodiments, the method further includes controlling the amount of the at least one organic substrate or the amount of the at least one organic product produced by adding at least one promoter inducer to the organic substrate culture solution or the organic product culture solution. In certain embodiments, the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof. In certain embodiments, the at least one promoter inducer includes lactose, xylose, IPTG, cold shock, heat shock, or a combination thereof.

In certain embodiments of methods herein, the at least one organic substrate includes AKG. In such embodiments, the at least one organic product includes ethylene. In certain embodiments, provided the at least one organic substrate culture impurity includes at least one volatile gas, the method further includes removing the at least one volatile gas through the volatile gas outlet.

In certain embodiments, the method includes recovering an amount of ethylene produced at a rate of from about 100 million pounds/year to about 1 billion pounds/year or more. In some embodiments, the method includes recovering an amount of ethylene produced at a rate of from about 200 million pounds/year to about 800 million pounds/year or more. In certain embodiments, the method includes recovering an amount of ethylene produced at a rate of from about 400 million pounds/year to about 600 million pounds/year or more. In certain embodiments, the method includes recovering an amount of ethylene produced at a rate of from about 0.13 to about 8.3 pounds per gallon of bioreactor culture per month. At scale, 0.13 pounds/gallon-bioreactor/month productivity can be translated to an industrial plant with up to 642 commercial scale (1,000,000 gallon) bioreactors for an annual production of about 1 billion pounds of ethylene or more. If productivity is increased to about 8.3 pounds/gallon-bioreactor/month, 10 commercial scale (1,000,000 gallon) bioreactors would be sufficient for an industrial plant with 1 billion pounds/year or more of ethylene production. In certain embodiments, the amount of ethylene produced contains an amount of volatile gas of about 1 mole percent or less. In certain embodiments, the amount of ethylene produced contains an amount of volatile gas of about 0.5 mole percent or less. In certain embodiments, the amount of ethylene produced contains an amount of volatile gas of about 0.25 mole percent or less.

Embodiments herein are directed to methods of producing an organic product, wherein the method includes providing a bioremediation system including: at least one bioreactor culture vessel; wherein the at least one bioreactor culture vessel contains an organic substrate culture solution; wherein the organic substrate culture solution contains a first recombinant microorganism; wherein the first recombinant microorganism has an improved organic substrate producing ability, expresses at least one organic substrate forming recombinant enzyme by expressing at least one non-native organic substrate forming enzyme nucleotide sequence, is capable of utilizing a carbon source to produce the organic substrate, and produces at least one organic substrate culture impurity, including at least one of a volatile gas, a liquid, and a solid; and wherein the at least one bioreactor culture vessel contains an organic product culture solution; wherein the organic product culture solution contains a second recombinant microorganism; wherein the second recombinant microorganism has an improved organic product producing ability, expresses at least one organic product forming enzyme by expressing at least one non-native organic product forming enzyme nucleotide sequence, and is capable of utilizing the organic substrate to produce the at least one organic product. In such embodiments, the organic substrate culture solution and the organic product culture solution are combined in one bioreactor culture vessel, wherein the non-native organic substrate forming recombinant enzyme expressing nucleotide sequence is inserted into a first microbial expression vector, wherein the non-native organic product forming enzyme expressing nucleotide sequence is inserted into a second microbial expression vector, and wherein the first and second microbial expression vector each includes at least one microbial expression promoter.

In such embodiments, the method includes providing a carbon source connected to the carbon source inlet; culturing the first recombinant microorganism in the first bioreactor culture vessel under conditions sufficient to produce an amount of the at least one organic substrate in the first bioreactor culture vessel; producing the amount of the at least one organic substrate by adding at least one promoter inducer to the organic substrate culture solution at a first time point; culturing the second recombinant microorganism in the second bioreactor culture vessel under conditions sufficient to produce an amount of the at least one organic product in the second bioreactor culture vessel; and producing the amount of the at least one organic product by adding at least one promoter inducer to the organic product culture solution at a second time point. Such embodiments can provide a benefit of separating the production of the at least one organic substrate culture impurity by the first recombinant microorganism, from the production of the at least one organic product by the second recombinant microorganism, by producing the at least one organic substrate and the at least one organic product at different time points, by adding at least one promoter inducer to the organic substrate culture solution or to the organic product culture solution at different time points.

In certain embodiments, the method includes lowering an amount of volatile gas in the second bioreactor culture vessel to from about 10% by volume to about 1% by volume or less, based a total internal volume of the second bioreactor culture vessel, before the second time point. In some embodiments, the method includes lowering an amount of volatile gas in the second bioreactor culture vessel to from about 5% by volume to about 0.5% by volume or less, based a total internal volume of the second bioreactor culture vessel, before the second time point. In certain embodiments, the method includes lowering an amount of volatile gas in the second bioreactor culture vessel to from about 1% by volume to about 0.1% by volume or less, based a total internal volume of the second bioreactor culture vessel, before the second time point. Such embodiments can provide a benefit of producing the at least one organic product containing safe levels of volatile gas. Such embodiments can provide a benefit of producing the at least one organic product containing volatile gas levels within regulatory limits.

Embodiments herein are directed to methods of producing ethylene. In certain embodiments, the method includes providing a biomanufacturing system including: a first bioreactor culture vessel including a carbon source inlet, a volatile gas outlet, and a power source; and a second bioreactor culture vessel including a fluid flow path connected to and between the first bioreactor culture vessel and the second bioreactor culture vessel, and an organic product outlet; wherein the first bioreactor culture vessel includes an organic substrate culture solution, and the second bioreactor culture vessel includes an organic product culture solution; wherein the organic substrate culture solution contains a first recombinant microorganism having an improved organic substrate producing ability, wherein the organic substrate includes AKG, wherein the first recombinant microorganism expresses at least one organic substrate forming recombinant enzyme by expressing at least one non-native organic substrate forming enzyme nucleotide sequence, wherein the organic substrate forming enzyme includes an AKG forming enzyme, wherein the first recombinant microorganism is capable of utilizing a carbon source to produce an amount of AKG, wherein the first recombinant microorganism produces at least one organic substrate culture impurity, including at least one of a volatile gas, a liquid, and a solid; wherein the volatile gas includes oxygen, wherein the organic product culture solution contains a second recombinant microorganism having an improved organic product producing ability, wherein the organic product includes ethylene, wherein the second recombinant organism expresses at least one organic product forming enzyme by expressing at least one non-native organic product forming enzyme nucleotide sequence, wherein the organic product forming enzyme includes EFE, and wherein the second recombinant organism is capable of utilizing AKG to produce an amount of ethylene. In certain embodiments, the method includes culturing the first recombinant microorganism in the first bioreactor culture vessel under conditions sufficient to produce an amount of AKG in the first bioreactor culture vessel; culturing the second recombinant microorganism in the second bioreactor culture vessel under conditions sufficient to produce an amount of EFE in the second bioreactor culture vessel; removing an amount of oxygen from the organic substrate culture solution through the volatile gas outlet; and removing an amount of EFE from the organic product culture solution through the organic product outlet. Such embodiments can provide a benefit of producing oxygen and ethylene in separate bioreactor culture vessels; in such embodiments, the co-production of oxygen and a volatile product such as ethylene can be reduced or eliminated. Such embodiments can also provide a benefit of producing a non-volatile organic substrate, such as AKG, that can be utilized for production of ethylene in the second bioreactor culture vessel.

### Additional Embodiments

Embodiment 1. A biomanufacturing system for producing an organic product comprising:

at least one bioreactor culture vessel;

wherein the at least one bioreactor culture vessel contains an organic substrate culture solution,

wherein the organic substrate culture solution contains a first recombinant microorganism having an improved organic substrate producing ability,

wherein the first recombinant microorganism expresses at least one organic substrate forming recombinant enzyme by expressing at least one non-native organic substrate forming enzyme nucleotide sequence,

wherein the first recombinant microorganism is capable of utilizing a carbon source to produce the organic substrate,

wherein the first recombinant microorganism produces at least one organic substrate culture impurity, including at least one of a volatile gas, a liquid, and a solid; and wherein the at least one bioreactor culture vessel contains an organic product culture solution,

wherein the organic product culture solution contains a second recombinant microorganism having an improved organic product producing ability,

wherein the second recombinant organism expresses at least one organic product forming enzyme by expressing at least one non-native organic product forming enzyme nucleotide sequence,

wherein the second recombinant organism is capable of utilizing the organic substrate to produce the at least one organic product.

Embodiment 2. The system of Embodiment 1 or any previous embodiment, wherein the at least one bioreactor culture vessel includes a first bioreactor culture vessel including a carbon source inlet, a volatile gas outlet, and a power source; and a second bioreactor culture vessel including a fluid flow path connected to and between the first bioreactor culture vessel and the second bioreactor culture vessel, and an organic product outlet;

wherein the first bioreactor culture vessel includes the organic substrate culture solution, and the second bioreactor culture vessel includes the organic product culture solution; or wherein the first bioreactor culture vessel or the second bioreactor culture vessel further comprises a biomass collection port.

Embodiment 3. The system of Embodiment 2 or any previous embodiment, wherein the carbon source includes carbon dioxide, carbon monoxide, an n-alkane, ethanol, a vegetable oil, glycerol, glucose, sucrose, a monosaccharide, a disaccharide, a polysaccharide, or a combination thereof; or

wherein the power source includes sunlight, a solar power source, an electrical power source, or a combination thereof; or

wherein the volatile gas includes oxygen, methane, or a combination thereof; or

wherein the at least one organic substrate includes alpha-ketoglutarate, sucrose, glucose, glycerol, a monosaccharide, a disaccharide, a polysaccharide, or a combination thereof; or

wherein the at least one organic product includes an alcohol, methanol, ethanol, propanol, butanol, ethane diol, an organic acid, propionic acid, acetic acid, an aldehyde, formaldehyde, a long chain fatty acid, an n-alkane, a hydrocarbon, or a combination thereof; or wherein the second bioreactor culture vessel further includes a carbon source inlet, a volatile gas outlet, a power source, or a combination thereof.

Embodiment 4. The system of Embodiment 1 or any previous embodiment, wherein the organic substrate culture solution and the organic product culture solution are combined in one bioreactor culture vessel; or

the organic substrate culture solution and the organic product culture are separated by a filter wherein the filter includes a pore size of from about 0.2 μm to about 10 μm or more; or wherein the system further comprises a carbonation unit, an amine stripper, an amine scrubber, a catalytic converter, a condenser, a compressor, a caustic tower, a dryer, or combinations thereof.

Embodiment 5. The system of Embodiment 1 or any previous embodiment, wherein the organic substrate includes alpha-ketoglutarate (AKG), wherein an amount of the at least one AKG forming enzyme produced by the first recombinant microorganism is greater than that produced relative to a control microorganism lacking a non-native AKG forming enzyme expressing nucleotide sequence; or

wherein the at least one organic product forming enzyme includes ethylene forming enzyme (EFE), and an amount of EFE produced by the second recombinant microorganism is greater than that produced relative to a control microorganism lacking a non-native EFE expressing nucleotide sequence.

Embodiment 6. The system of Embodiment 5 or any previous embodiment, wherein the first recombinant microorganism expresses at least one alpha-ketoglutarate permease protein (AKGP) by expressing at least one non-native AKGP forming nucleotide sequence.

Embodiment 7. The system of Embodiment 5 or any previous embodiment, wherein the at least one AKG forming enzyme includes an isocitrate dehydrogenase (ICD) protein, a glutamate dehydrogenase (GDH) protein, or a combination thereof.

Embodiment 8. The system of Embodiment 7 or any previous embodiment, wherein the first recombinant microorganism expresses an ICD protein having an amino acid sequence at least 95% identical to SEQ ID NO: 1 by expressing a non-native ICD protein nucleotide sequence having a nucleotide sequence at least 95% identical to SEQ ID NO: 2; or

the first recombinant microorganism expresses a GDH protein having an amino acid sequence at least 95% identical to SEQ ID NO: 5 by expressing a non-native GDH protein nucleotide sequence having a nucleotide sequence at least 95% identical to SEQ ID NO: 6, or

a combination thereof; or wherein the second recombinant microorganism expresses an EFE protein having an amino acid sequence at least 95% identical to SEQ ID NO: 7 by expressing a non-native EFE protein nucleotide sequence having a nucleotide sequence at least 95% identical to SEQ ID NO: 8.

Embodiment 9. The system of Embodiment 1 or any previous embodiment, wherein the first recombinant microorganism includes a microorganism selected from the group consisting of a photosynthetic bacteria, a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, Arthrobacter paraffineus, Pseudomonas fluorescens, Serratia marcescens, Bacillus metatherium, Candida paludigena, Pichia inositovora, Torulopsis glabrata, Candida lipolytica, Yarrowia lipolytica, Saccharomyces cereviciae, Anabaena sp., Aspergillus sp., Bacillus subtilis, Chlorella sp., Lactobacillus sp., algae, microalgae, electrosynthesis bacteria, a photosynthetic microorganism, yeast, filamentous fungi, and a plant cell; or wherein the second recombinant microorganism includes a microorganism selected from the group consisting of Escherichia, Escherichia coli, Geobacteria, Arthrobacter paraffineus, Pseudomonas fluorescens, Serratia marcescens, Bacillus metatherium, Candida paludigena, Pichia inositovora, Torulopsis glabrata, Candida lipolytica, Yarrowia lipolytica, Saccharomyces cereviciae, Anabaena sp., Aspergillus sp., Bacillus subtilis, Chlorella sp., Lactobacillus sp., an anaerobic bacteria, and Bacillus subtilis.

Embodiment 10. The system of Embodiment 1 or any previous embodiment, wherein the first recombinant microorganism includes a delta-glgc mutant microorganism lacking expression of a glucose-1-phosphate adenylyltransferase protein; or wherein the first recombinant microorganism expresses a sucrose synthase protein having an amino acid sequence at least 95% identical to SEQ ID NO: 9 by expressing a non-native sucrose synthase protein nucleotide sequence having a nucleotide sequence at least 95% identical to SEQ ID NO: 10; or wherein the first recombinant microorganism expresses a sucrose phosphate synthase protein having an amino acid sequence at least 95% identical to SEQ ID NO: 11 by expressing a non-native sucrose phosphate synthase nucleotide sequence having a nucleotide sequence at least 95% identical to SEQ ID NO: 12.

Embodiment 11 The system of Embodiment 5 or any previous embodiment, wherein an amount of at least one AKG forming enzyme produced by the first recombinant microorganism is from about 5% to about 200% or more greater than that produced relative to the control microorganism lacking the non-native AKG forming enzyme expressing nucleotide sequence; or

wherein an amount of EFE protein produced by the second recombinant microorganism is from about 5% to about 200% or more greater than that produced relative to the control microorganism lacking the non-native EFE expressing nucleotide sequence; or

wherein an amount of EFE protein produced by the second recombinant microorganism is from about 20 grams to about 100 grams per liter or more of organic product culture solution.

Embodiment 12. The system of Embodiment 5 or any previous embodiment, wherein the second recombinant microorganism includes E. Coli, and an amount of EFE protein produced by the second recombinant microorganism is from about 30% to about 80% or more of a total cellular amount of protein of the second recombinant microorganism; or

a production rate of the at least one organic product produced by the second recombinant microorganism is from about 100 million pounds/year to about 1 billion pounds/year or more; or wherein a cell population concentration of the second recombinant microorganism ranges from about 107 to about 1013 cells per milliliter, or a dry cell weight per liter of organic product culture solution of from about 100 grams to about 300 grams dry cell weight per liter.

Embodiment 13. The system of Embodiment 5 or any previous embodiment, wherein the non-native AKG forming enzyme expressing nucleotide sequence or the non-native EFE expressing nucleotide sequence is inserted into a microbial expression vector, wherein the microbial expression vector includes a bacterial vector plasmid, a nucleotide guide of a homologous recombination system, an antibiotic-resistant system, an aid system for protein purification and detection, a CRISPR CAS system, a phage display system, or a combination thereof.

Embodiment 14. The system of Embodiment 5 or any previous embodiment, wherein the EFE expressing nucleotide sequence has a copy number in the microbial expression vector of from about 2 to about 500; or wherein the microbial expression vector includes at least one microbial expression promoter.

Embodiment 15. The system of Embodiment 14 or any previous embodiment, wherein the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof.

Embodiment 16. A method of producing an organic product comprising:

providing a biomanufacturing system as in Embodiment 2 or any previous embodiment;

culturing the first recombinant microorganism in the first bioreactor culture vessel under conditions sufficient to produce an amount of the at least one organic substrate in the first bioreactor culture vessel; and

culturing the second recombinant microorganism in the second bioreactor culture vessel under conditions sufficient to produce an amount of the at least one organic product in the second bioreactor culture vessel.

Embodiment 17. The method of Embodiment 16 or any previous embodiment, further comprising:

removing an amount of the at least one volatile gas from the organic substrate culture solution through the volatile gas outlet;

removing an amount of the at least one organic product from the organic product culture solution through the organic product outlet;

provided the carbon source includes carbon dioxide, feeding an amount of the carbon dioxide from a carbon dioxide source into the organic substrate culture solution through the carbon source inlet;

maintaining a pH level of the organic substrate culture solution and the organic product culture solution from about 5.0 to about 8.5;

maintaining the organic substrate culture solution and the organic product culture solution at a temperature of from about 25 degrees Celsius to about 70 degrees Celsius;

provided the first bioreactor culture vessel or the second bioreactor culture vessel includes a biomass collection port, collecting an amount of biomass produced by the first recombinant microorganism or the second recombinant microorganism through the biomass collection port; or

maintaining an amount of volatile gas in the second bioreactor culture vessel of from about 10% by volume to about 1% by volume or less, based on a total internal volume of the second bioreactor culture vessel.

Embodiment 18. The method of Embodiment 16 or any previous embodiment, wherein the non-native organic substrate forming recombinant enzyme expressing nucleotide sequence or the non-native organic product expressing nucleotide sequence is inserted into a microbial expression vector, wherein the at least one microbial expression vector includes at least one microbial expression promoter, further comprising:

controlling the amount of the at least one organic substrate or the amount of the at least one organic product produced by adding at least one promoter inducer to the organic substrate culture solution or the organic product culture solution.

Embodiment 19. The method of Embodiment 18 or any previous embodiment, wherein the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof; and the at least one promoter inducer includes lactose, xylose, IPTG, cold shock, heat shock, or a combination thereof.

Embodiment 20. The method of Embodiment 16 or any previous embodiment, further comprising:

provided the at least one organic substrate culture impurity includes at least one volatile gas, removing the at least one volatile gas through the volatile gas outlet; or recovering an amount of at least one organic product produced at a rate of from about 100 million pounds/year to about 1 billion pounds/year or more; or wherein the amount of the at least one organic product produced contains an amount of volatile gas of about 1 mole percent or less.

Embodiment 21. A method of producing an organic product comprising: providing a bioremediation system as in Embodiment 1 or any previous embodiment, wherein the organic substrate culture solution and the organic product culture solution are combined in one bioreactor culture vessel, wherein the non-native organic substrate forming recombinant enzyme expressing nucleotide sequence is inserted into a first microbial expression vector, wherein the non-native organic product forming enzyme expressing nucleotide sequence is inserted into a second microbial expression vector, wherein the first and second microbial expression vector each includes at least one microbial expression promoter,

providing a carbon source connected to the carbon source inlet;

culturing the first recombinant microorganism in the first bioreactor culture vessel under conditions sufficient to produce an amount of the at least one organic substrate in the first bioreactor culture vessel;

producing the amount of the at least one organic substrate by adding at least one promoter inducer to the organic substrate culture solution at a first time point;

culturing the second recombinant microorganism in the second bioreactor culture vessel under conditions sufficient to produce an amount of the at least one organic product in the second bioreactor culture vessel; and

producing the amount of the at least one organic product by adding at least one promoter inducer to the organic product culture solution at a second time point.

Embodiment 22. The method of Embodiment 21 or any previous embodiment, further comprising lowering an amount of volatile gas in the second bioreactor culture vessel to from about 10% by volume to about 1% by volume or less, based on a total internal volume of the second bioreactor culture vessel, before the second time point.

### EXAMPLES

### Example 1. Cloning of Alpha-Ketoglutarate Synthesis Enzymes into Cyanobacteria

Alpha-ketoglutarate (aKG) is produced by oxidative decarboxylation of isocitrate by isocitrate dehydrogenase (ICD), or by oxidative deamination of glutamate by glutamate dehydrogenase (GDH). Target enzymes for cloning and aKG production in Cyanobacteria include ICD Enzyme: 1.1.1.42, coding sequence of P. fluorescens ICD (SEQ ID NO: 1, SEQ ID NO: 2), ICD Enzyme: 1.1.1.42, coding sequence of Synechococcus elongatus PCC794 (SEQ ID. NO: 3, SEQ ID NO: 4), and GDH Enzyme: 1.4.1.2, coding sequence of P. Fluorescens (SEQ ID NO: 5, SEQ ID NO: 6).

The ICD and GDH genes are synthesized as gBlocks cloned into a pSyn6 plasmid construct (pSyn6_ICD and pSyn6_GDH). For cloning into a pSyn6 plasmid, the S. elongatus ICD coding sequence is flanked by an N-terminal HindIII and C-terminal BamHI recognition sites (SEQ ID NO: 4). Using the plasmid constructs, the ICD and GDH genes are cloned into an unmodified S. elongatus or an S. elongatus Δglgc mutant strain (see Example 2). Between 1-3 copies of the target genes will be transformed. Cloning of the ICD and GCH genes are confirmed by PCR and sequencing. aKG synthesis and quantification are evaluated by SDS-PAGE, Western Blot, and Ethylene production assays.

Culture growth rate and culture conditions are evaluated pursuant to scale up of aKG production.

### Example 2. Engineering Cyanobacteria for Secretion of Alpha-Ketoglutarate

Creating glycogen mutant strains of Cyanobacteria changes the bacteria's pathway to produce higher concentrations of keto acids such as aKG.

Glycogen mutant Cyanobacteria are generated by creating glycogen deficient strains via mutations of the glgc gene (Δglgc). An Ampicillin Resistance (AmpR) gene are synthesized as gBlocks and incorporated into a plasmid construct. The plasmid construct is transformed into wild type Cyanobacteria (Synechocystis, Synechococcus elongatus 2973, Synechococcus elongatus 2434). A portion of the wild type glgc gene is replaced by the AmpR gene to create the mutant strains. The Δglgc mutant strains is confirmed by growth in AmpR containing media, followed by PCR and sequencing.

### Example 3. Sucrose Production from Carbon Dioxide

Cyanobacteria (Synechococcus elongatus, Synechocystis) are engineered to produce sucrose as a substrate for the growth of microorganisms producing ethylene downstream (E. coli).

Engineering of Synechococcus elongatus PCC 7942 is including activation of one gene (cscB) and deletion of one gene (GlgC). Yield of sucrose generated in the engineered bacteria from 15 to 31.5 pounds/gallon-bioreactor/month.

Large scale production of sucrose is performed using a 1-ha photobioreactor (640,000 liters), a 2% carbon dioxide supply, a growing season of 300 days, light of 8 hours per day (65 uE m² s⁻¹), and a yield of up to 55 metric tons sucrose/year.

### Example 4. Cloning of Ethylene Forming Enzyme into E. coli

E. coli is chosen for ethylene production for its fast growth rate, and the availability of extensive tools for genetic modifications. The current plasmid allows producing ethylene forming enzyme at a very high yield.

A polynucleotide coding sequence of EFE which initially is adapted from the Pseudomonas savastanoi pv. Phaseolicola EFE protein is selected and gene adapted for expression in E. coli (GenBank: KPB44727.1, SEQ ID NO:6). A synthetic DNA construct encoding EFE enzyme is synthesized and cloned into the pET-30a(+) vector plasmid. The corresponding nucleotides sequences are codon adapted for expression in E. coli (SEQ ID NO: 5), containing an optional His tag at the C-terminal end followed by a stop codon and HindIII site. An NdeI site is used for cloning at the 5-prime end, where the NdeI site contains an ATG start codon. E. coli BL21(DE3) competent cells are transformed with the recombinant plasmid.

An Ampicillin cassette is activated by an IPTG inducible promoter (pTrc) in the presence of a LacI gene; the LacI gene is regulated by a LacIq promoter (SEQ ID NO: 13).

E. coli strain BL21(DE3): Doubling time (20 min), cells sink to bottom of bioreactor unless being stirred.
Theoretical maximum cell density: 50 g dry cell weight/L or 1×10¹³ viable bacteria/L. in normal condition: 1×10¹⁰ viable bacteria/L. Use of rich media can push higher cell density.
Culture pH: 7.5-8.5
Plasmid: low (2-4), medium (15-60) and high (50-500) copy number
Promoter/inducer for protein production
Lac promoter/lactose: The presence of glucose will compete with lactose and negative impact the protein production
T7 promoter/IPTG: Target protein represents 50% of total cell protein
CspA promoter/cold shock: Protein expression as temperature decrease from 37° C.-15° C.
λpL-λcI promoter/heat shock: Protein expression as temperature increase from 37° C.-42° C.
psbA promoter: No inducer required
Current yield of ethylene production from E. coli: 188 nmol ethylene/OD600/mL

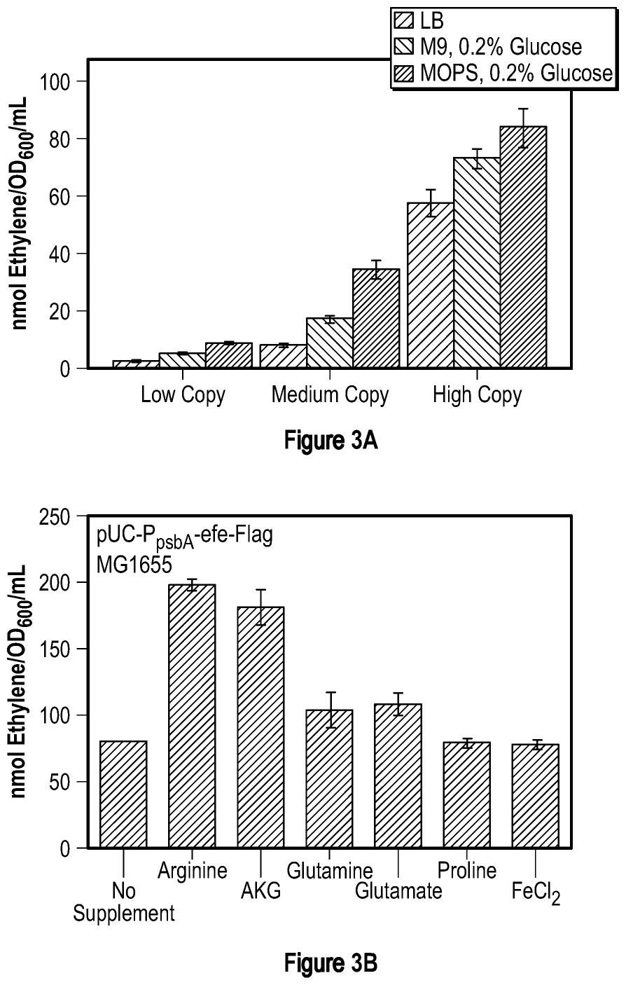

A pilot expression of EFE (about 44.5 kDa)_BL21(DE3) was conducted with either no EFE, a Low Copy number construct (pRK290-PpsbA-efe-Flag), a Medium Copy number construct (pBBR1-PpsbA-efe-Flag), or a High Copy number construct (pUC-PpsbA-efe-Flag). SDS-PAGE and Western blotting were used to monitor the EFE protein expression in the various Copy Number constructs (GenScript USA, Inc., Piscataway, N.J.) in cultures grown in Luria Broth, M9 medium with 0.2% glucose, or MOPS medium with 0.2% glucose. For the blots, alpha-GroEL expression was measured as a positive control. The levels of EFE protein produced by the Control, Low Copy, Medium Copy, and High Copy constructs in cultures grown in various culture media is shown in FIG. 3A.

The yield of ethylene from the pUC-PpsbA-efe-Flag MB1655 construct was evaluated in growth in culture media with either no supplement, or with various growth supplements added to the culture media (FIG. 3B).

### Example 5: Synthetic Production of Conjugated Alkenes, Polyenes and Alkanes in Recombinant Microorganisms

Conjugated alkenes (including dienes and polyenes) and alkanes are important commodity products with several applications in the polymers industry, and biotechnology. Renewable production of conjugated alkenes and alkanes by synthetic biology can be an alternative approach to production of petroleum-based chemicals. Short alkanes and alkenes are volatile gases. Examples of these volatile gases include, Ethene, Ethylene, Propene, Butene, methane, Ethane, Propane, butane, or a combination thereof.

Natural processes for production of alkenes and alkanes are extremely slow. Here, we applied a genetic modification approach for production of conjugated alkenes and alkanes. To this end, overexpression of critical enzymatic pathways involved in biosynthesis of Alkenes and alkanes were done. Five enzymatic pathways for biosynthesis of alkenes and alkanes were defined through bioinformatics studies. Free fatty acids or carbohydrates (sugars) can be used as feedstocks for these reactions. Four enzymatic pathways that convert free acids or their derivatives to alkanes or alkenes are including:
1—Decarboxylation of fatty aldehydes, 2—decarboxylation of fatty acids, 3—head to head hydrocarbon biosynthesis, 4—Polyketide synthase (PKS) pathway. Among these pathways, decarboxylation of fatty aldehydes has the highest efficiency. Aldehyde decarboxylases (ADs) play a critical role in this pathway. Aldehyde decarboxylases convert the fatty aldehydes to alkanes and/or alkenes. Other critical enzymes are including fatty acryl carrier protein (ACP) reductase (AAR) or Fatty acid reductase (FAR).
   Decarboxylation of fatty acids (for production of alkenes/alkanes) is regulated by three critical enzymes including Ole T, UndA and UndB. Ole ABCD gene are critical for head to head hydrocarbon biosynthesis.
   Critical enzymes of Polyketide (PKS) pathway are including 3-B-keto-acyl synthases (KS), acyl-transferase (AT), acyl carrier protein (ACP), B-keto reductase (KR), dehydratase (DH), enyl reductase (ER).

In order to produce alkanes and alkenes, the above mentioned genetic pathways were modeled. E. coli, was used as a host microorganism. Gene constructs were made using pUC19 (high copy number) as an expression vector and under an IPTG-inducible promoter. Expression of each gene are confirmed by colony growth on agar media supplemented with ampicillin, IPTG and X-gal, PCR and gel electrophoresis. Gas chromatography (GC) and HPLC are used for detection of products in the gas and liquid phase respectively. The psbA promoter is a continuous expression promoter. E. coli BL21 (DE3), DH5alpha, or MG1655 cell lines are used as host.

Cell culture adaptations were done by cultivation of E. coli in different media, including LB, and MOPS. Composition of MOPS media is defined as the list, below.

| Media | MOPS | |
|---|---|---|
| Glucose | 4 | g/L |
| IPTG | 0.5 | mM |
| Arginine | 3 | mM |
| AKG | 2 | mM |

### Induction Induced at the Start

Below please see the list of genes that are involved in the production of alkanes and alkenes and their excision numbers.

Aldehyde decarboxylases (ADs), fatty acryl carrier protein (ACP) reductase (AAR) or Fatty acid reductase (FAR), Saccharomyces cerevisiae S288C tetrafunctional fatty acid synthase subunit FAS1 (FAS1), partial mRNA NCBI Reference Sequence: NM_001179748.1 (SEQ ID NO. 14)

Ole T, UndA and UndB.

Ole ABCD gene

3-B-keto-acyl synthases (KS), acyl-transferase (AT), Arabidopsis thaliana HXXXD-type acyl-transferase family protein (CER2), mRNA NCBI Reference Sequence: NM_118584.3 (SEQ ID NO. 15)

acyl carrier protein (ACP), Leptomonas pyrrhocoris putative mitochondrial acyl carrier protein, putative (ACP) mRNA NCBI Reference Sequence: XM_015809471.1 (SEQ ID NO. 16)

B-keto reductase (KR), dehydratase (DH), enyl reductase (ER).

beta-hydroxyacyl-acyl carrier protein dehydratase/isomerase [Escherichia coli str. K-12 substr. MG1655] NCBI Reference Sequence: NP_415474.1 (SEQ ID NO. 17)

### Example 6. Lab Scale Experimental Procedures

1. Tailored-designed DNA constructs will be generated that encode the critical intermediates of a recombinant alkene, polyene, alkane, polyol, alcohol and organic acid biosynthesis pathway. 2. Carefully selected photosynthetic first recombinant microorganisms will then be expanded for cloning and gene expression of organic substrates including alpha ketoglutarate, sucrose, glucose, fructose, xylose, galactose, glycerol, fatty acids, etc. 3. Carefully selected second recombinant microorganisms will then be expanded for cloning and gene expression of organic product forming enzymes. 4. Genetic and metabolic engineering of microorganisms will then be performed for the continuous production of the organic products. 5. Bioengineering microorganisms will then be selected and expanded in a photobioreactor and bioreactor system. 6. Bioreactor culture conditions (including CO₂ concentration, light exposure time and wave-length, temperature, and pH) will be adapted. 7. Samples will be collected and analyzed by HPLC to measure organic product synthesis. 8. Organic product production in genetically engineered microorganisms will be adapted. 9. Organic product production processes will be scaled up from the test tube scale to 1-liter, 10 liters and 100 liters bioreactors. Scale ups will be done with the ratio of 1 to 10 at each stage.

### APPENDIX

SEQ ID NO: 1
MGYKKIQVPAVGDKITVNADHSLNVPDNPIIPFIE
GDGIGVDVSPVMIKVVDAAVEKAYGGKRKISWMEV
YAGEKATQVYDQDTWLPQETLDAVKDYVVSIKGPL
TTPVGGGIRSLNVALRQQLDLYVCLRPVVWFEGVP
SPVKKPGDVDMVIFRENSEDIYAGIEWKAGSPEAT
KVIKFLKEEMGVTKIRFDQDCGIGIKPVSKEGTKR
LVRKALQYVVDNDRKSLTIVHKGNIMKFTEGAFKD
WGYEVAKEEFGAELLDGGPWMKFKNPKTGREVVVK
DAIADAMLQQILLRPAEYDVIATLNLNGDYLSDAL
AAEVGGIGIAPGANLSDTVAMFEATHGTAPKYAGK
DQVNPGSVILSAEMMLRHLGWTEAADLIIKGTNGA
IKAKTVTYDFERLMEGATLVSSSGFGEALIKHM
SEQ ID NO: 2
ATGGGTTACAAGAAGATTCAGGTTCCAGCCGTCGG
CGACAAAATCACCGTCAACGCAGACCATTCTCTCA
ATGTCCCTGATAACCCGATCATTCCCTTCATCGAA
GGTGACGGCATTGGCGTCGACGTCAGCCCTGTGAT
GATCAAAGTGGTTGATGCTGCCGTAGAGAAAGCCT
ACGGGGGTAAGCGCAAGATTTCCTGGATGGAGGTT
TATGCTGGCGAAAAAGCAACTCAGGTCTATGACCA
GGACACCTGGCTGCCCCAGGAAACCCTGGACGCGG
TCAAGGATTACGTGGTCTCCATCAAAGGCCCGCTG
ACCACTCCGGTCGGTGGCGGCATCCGTTCCCTCAA
CGTCGCCCTGCGCCAACAGCTCGATCTCTATGTCT
GCCTTCGCCCTGTGGTGTGGTTCGAAGGTGTGCCG
AGCCCGGTGAAAAAGCCTGGCGACGTCGACATGGT
GATCTTCCGCGAGAACTCCGAAGACATTTATGCCG
GTATCGAATGGAAAGCCGGCTCCCCTGAGGCCACC
AAGGTCATCAAATTCCTGAAAGAAGAAATGGGCGT
CACCAAGATCCGTTTCGACCAGGATTGCGGCATCG
GCATCAAGCCGGTTTCCAAAGAAGGCACCAAGCGT
CTGGTGCGCAAGGCGCTGCAATACGTGGTGGACAA
CGACCGCAAGTCGCTGACCATCGTGCACAAGGGCA
ACATCATGAAATTCACCGAAGGTGCCTTCAAGGAC
TGGGGCTACGAGGTGGCGAAGGAAGAATTCGGCGC
CGAGCTGCTCGATGGCGGCCCATGGATGAAATTCA
AGAACCCGAAAACCGGCCGCGAAGTCGTCGTCAAG
GACGCCATCGCCGACGCCATGCTCCAGCAGATCCT
GCTGCGTCCGGCCGAATACGATGTGATCGCCACCC
TCAACCTCAACGGTGACTACCTGTCCGACGCCCTG
GCGGCGGAAGTGGGCGGTATCGGTATCGCGCCGGG
TGCCAACCTGTCCGACACCGTAGCCATGTTCGAGG
CGACCCACGGTACTGCGCCGAAATATGCCGGCAAG
GACCAGGTCAACCCGGGTTCGGTGATTTTGTCGGC
GGAAATGATGCTGCGCCACCTGGGCTGGACCGAGG
CGGCCGACCTGATCATCAAGGGCACCAATGGCGCC
ATCAAGGCCAAGACCGTGACCTACGACTTCGAACG
TCTGATGGAAGGCGCCACACTGGTGTCTTCTTCGG
GCTTCGGTGAAGCGCTGATCAAGCACATGTAA
SEQ ID NO: 3
MYEKIQPPSEGSKIRFEAGKPIVPDNPIIPFIRGD
GTGVDIWPATERVLDAAVAKAYGGQRKITWFKVYA
GDEACDLYGTYQYLPEDTLTAIREYGVAIKGPLTT
PIGGGIRSLNVALRQIFDLYACVRPCRYYTGTPSP
HRTPEQLDVVVYRENTEDIYLGIEWKQGDPTGDRL
IKLLNEDFIPNSPSLGKKQIRLDSGIGIKPISKTG
SQRLIRRAIEHALRLEGRKRHVTLVHKGNIMKFTE
GAFRDWGYELATTEFRTDCVTERESWILANQESKP
DLSLEDNARLIEPGYDAMTPEKQAAVVAEVKAVLD
SIGATHGNGQWKSKVLVDDRIADSIFQQIQTRPGE
YSVLATMNLNGDYISDAAAAVVGGLGMAPGANIGD
EAAIFEATHGTAPKHAGLDRINPGSVILSGVMMLE
YLGWQEAADLITKGISQAIANREVTYDLARLMEPA
VDQPLKCSEFAEAIVKHFDD
SEQ ID NO: 4
AGGGCAAGCTTATGTACGAGAAGATTCAACCCCCT
AGCGAAGGCAGCAAAATTCGCTTTGAAGCCGGCAA
GCCGATCGTTCCCGACAACCCGATCATTCCCTTCA
TTCGTGGTGACGGCACTGGCGTTGATATCTGGCCC
GCAACTGAGCGCGTTCTCGATGCCGCTGTCGCTAA
AGCCTATGGCGGTCAGCGCAAAATCACTTGGTTCA
AAGTCTACGCGGGTGATGAAGCCTGCGACCTCTAC
GGCACCTACCAATATCTGCCTGAAGATACGCTGAC
AGCGATCCGCGAGTACGGCGTGGCAATCAAAGGCC
CGCTGACGACGCCGATCGGTGGTGGCATTCGATCG
CTGAACGTGGCGCTACGGCAAATCTTCGATCTCTA
TGCCTGCGTCCGCCCCTGTCGCTACTACACCGGCA
CACCCTCGCCCCACCGCACGCCCGAACAACTCGAT
GTGGTGGTCTACCGCGAAAACACCGAGGATATCTA
CCTCGGCATCGAATGGAAGCAAGGTGATCCCACCG
GCGATCGCCTGATCAAGCTGCTGAACGAGGACTTC
ATTCCCAACAGCCCCAGCTTGGGTAAAAAGCAAAT
CCGTTTGGATTCCGGCATTGGTATTAAGCCGATCA
GTAAAACGGGTAGCCAGCGTCTGATTCGTCGTGCG
ATCGAGCATGCCCTACGCCTCGAAGGCCGCAAGCG
ACATGTCACCCTTGTCCACAAGGGCAACATCATGA
AGTTCACGGAAGGTGCTTTCCGGGACTGGGGCTAT
GAACTGGCCACGACTGAGTTCCGAACCGACTGTGT
GACTGAACGGGAGAGCTGGATTCTTGCCAACCAAG
AAAGCAAGCCGGATCTCAGCTTGGAAGACAATGCG
CGGCTCATCGAACCTGGCTACGACGCGATGACGCC
CGAAAAGCAGGCAGCAGTGGTGGCTGAAGTGAAAG
CTGTGCTCGACAGCATCGGCGCCACCCACGGCAAC
GGTCAGTGGAAGTCTAAGGTGCTGGTTGACGATCG
CATTGCTGACAGCATCTTCCAGCAGATTCAAACCC
GCCCGGGTGAATACTCGGTGCTGGCGACGATGAAC
CTCAATGGCGACTACATCTCTGATGCAGCGGCGGC
GGTGGTCGGTGGCCTGGGCATGGCCCCCGGTGCCA
ACATTGGCGACGAAGCGGCGATCTTTGAAGCGACC
CACGGCACAGCGCCCAAGCACGCTGGCCTCGATCG
CATTAACCCCGGCTCGGTCATCCTCTCCGGCGTGA
TGATGCTGGAGTACCTAGGCTGGCAAGAGGCTGCT
GACTTGATCACCAAGGGCATCAGCCAAGCGATCGC
TAACCGTGAGGTCACCTACGATCTGGCTCGGTTGA
TGGAACCGGCGGTTGATCAACCACTCAAGTGCTCG
GAATTTGCCGAAGCCATCGTCAAGCATTTCGACGA
TTAGGGATCCAGCGC
SEQ ID NO. 5
MAFFTAASKADFQHQLQAALAQHISEQALPQVALF
AEQFFGIISLDELTQRRLSDLAGCTLSAWRLLERF
DHAQPQVRVYNPDYERHGWQSTHTAVEVLHHDLPF
LVDSVRTELNRRGYSIHTEQTTVLSVRRGSKGELL
EILPKGTTGEGVLHESLMYLEIDRCANAAELNVLS
KELEQVLGEVRVAVSDFEPMKAKVQEILTKLDNSA
FAVDADEKNEIKSFLEWLVGNHFTFLGYEEFTVVD
QADGGHIEYDQNSFLGLTKMLRTGLTNEDRHIEDY
AVKYLREPTLLSFAKAAHPSRVHRPAYPDYVSIRE
IDADGKVIKEHRFMGLYTSSVYGESVRVIPFIRRK
VEEIERRSGFQAKAHLGKELAQVLEVLPRDDLFQT
PVDELFSTVMSIVQIQERNKIRVFLRKDPYGRFCY
CLAYVPRDIYSTEVRQKIQQVLMERLKASDCEFWT
FFSESVLARVQLILRVDPKNRIDIDPLQLENEVIQ
ACRSWQDDYAALTVETTGEANGTNVLADFPKGFPA
GYRERFAAHSAVVDMQHLLNLSEKKPLAMSFYQPL
ASGPRELHCKLYHADTPLALSDVLPILENLGLRVL
GEFPYRLRHTNGREFWIHDFAFTAAEGLDLDIQQL
NDTLQDAFVHIVRGDAENDAFNRLVLTAGLPWRDV
ALLRAYARYLKQIRLGFDLGYIASTENNHTDIARE
LTRLFKTRFYLARKLGSEDLDDKQQRLEQAILTAL
DDVQVLNEDRILRRYLDLIKATLRTNFYQTDANGQ
NKSYFSFKFNPHLIPELPKPVPKFEIFVYSPRVEG
VHLRFGNVARGGLRWSDREEDFRTEVLGLVKAQQV
KNSVIVPVGAKGGFLPRRLPLGGSRDEIAAEGIAC
YRIFISGLLDITDNLKDGKLVPPANVVRHDDDDPY
LVVAADKGTATFSDIANGIAIDYGFWLGDAFASGG
SAGYDHKKMGITAKGAWVGVQRHFRERGINVQEDS
ITVVGVGDMAGDVFGNGLLMSDKLQLVAAFNHLHI
FIDPNPNPATSFAERQRMFDLPRSAWSDYDTSIMS
EGGGIFSRSAKSIAISPQMKERFDIQADKLTPTEL
LNALLKAPVDLLWNGGIGTYVKASTESHADVGDKA
NDALRVNGNELRCKVVGEGGNLGMTQLGRVEFGLN
GGGSNTDFIDNAGGVDCSDHEVNIKILLNEVVQAG
DMTDKQRNQLLASMTDEVGGLVLGNNYKQTQALSL
AARRAYARIAEYKRLMSDLEGRGKLDRAIEFLPTE
EQLAERVAEGHGLTRPELSVLISYSKIDLKEQLLG
SLVPDDDYLTRDMETAFPPTLVSKFSEAMRRHRLK
REIVSTQIANDLVNHMGITFVQRLKESTGMTPANV
AGAYVIVRDIFHLPHWFRQIEALDYQVSADVQLEL
MDELMRLGRRATRWFLRARRNEQNAARDVAHFGPH
LKELGLKLDELLSGEIRENWQERYQAYVAAGVPEL
LARMVAGTTHLYTLLPIIEAADVTGQDPAEVAKAY
FAVGSALDITWYISQISALPVENNWQALAREAFRD
DVDWQQRAITIAVLQAGGGDSDVETRLALWMKQND
AMIERWRAMLVEIRAASGTDYAMYAVANRELNDVA
LSGQAVVPAAATAELELA
SEQ ID NO. 6
ATGGCGTTCTTCACCGCAGCCAGCAAAGCCGACTT
CCAGCACCAACTGCAAGCGGCACTGGCGCAGCACA
TCAGTGAACAGGCACTGCCACAAGTGGCGCTGTTC
GCTGAACAATTCTTCGGCATCATTTCCCTGGACGA
GCTGACCCAACGTCGCCTCTCCGACCTCGCTGGCT
GTACTCTCTCTGCGTGGCGCCTGCTTGAGCGCTTC
GATCACGCGCAACCGCAAGTGCGCGTCTACAACCC
CGATTACGAACGTCACGGCTGGCAGTCGACCCACA
CCGCGGTCGAAGTGCTGCACCACGACTTGCCGTTC
CTGGTGGACTCGGTGCGTACCGAGCTGAACCGTCG
CGGCTACAGCATCCACACCCTGCAGACCACCGTGC
TGAGCGTGCGTCGTGGCAGCAAGGGCGAATTGCTG
GAAATCCTGCCAAAAGGCACCACCGGCGAAGGCGT
TCTGCACGAGTCGCTGATGTACCTGGAAATCGACC
GCTGCGCCAATGCGGCCGAATTGAATGTGCTGTCC
AAGGAACTGGAGCAGGTCCTGGGTGAAGTCCGCGT
CGCGGTCTCCGATTTCGAGCCGATGAAGGCCAAGG
TGCAGGAAATCCTCACCAAGCTCGATAACAGCGCA
TTCGCCGTCGATGCCGACGAAAAGAATGAAATCAA
GAGCTTCCTGGAATGGCTGGTGGGCAACCACTTCA
CCTTCCTCGGCTACGAAGAGTTCACCGTTGTCGAT
CAGGCCGATGGCGGCCACATCGAATACGACCAGAA
CTCCTTCCTCGGCCTGACCAAGATGCTGCGCACCG
GTCTGACCAACGAAGACCGCCACATCGAAGACTAT
GCCGTGAAGTACCTGCGCGAACCGACACTGCTGTC
GTTCGCCAAGGCGGCGCATCCGAGCCGCGTGCACC
GTCCGGCCTACCCGGACTACGTGTCGATCCGCGAA
ATCGATGCCGACGGCAAAGTGATCAAGGAACACCG
CTTCATGGGCCTGTACACCTCGTCGGTGTATGGCG
AAAGCGTGCGTGTCATCCCGTTCATCCGCCGCAAG
GTCGAGGAAATCGAGCGTCGCTCCGGCTTCCAGGC
CAAGGCTCACCTGGGCAAGGAACTGGCTCAGGTTC
TGGAAGTGCTGCCGCGTGACGATCTGTTCCAGACC
CCGGTCGACGAACTGTTCAGCACCGTGATGTCGAT
CGTGCAGATCCAGGAACGCAACAAGATCCGCGTGT
TCCTGCGTAAAGACCCGTACGGTCGTTTCTGCTAC
TGCCTGGCCTACGTGCCGCGTGACATCTACTCCAC
CGAAGTTCGCCAGAAGATCCAGCAAGTGCTGATGG
AGCGCCTGAAAGCCTCCGACTGCGAATTCTGGACG
TTCTTCTCCGAGTCCGTGCTGGCCCGCGTGCAACT
GATCTTGCGCGTCGACCCGAAAAACCGCATCGACA
TCGACCCGCTGCAACTGGAAAACGAAGTGATCCAG
GCCTGCCGCAGCTGGCAGGACGACTACGCTGCCCT
GACCGTTGAAACCTTCGGCGAAGCCAACGGCACCA
ACGTGTTGGCCGACTTCCCGAAAGGCTTCCCGGCC
GGCTACCGCGAGCGTTTCGCAGCGCATTCGGCCGT
GGTCGACATGCAGCACTTGCTCAATCTGAGCGAGA
AAAAGCCGCTGGCCATGAGCTTTTACCAGCCGCTG
GCCTCCGGCCCACGCGAGCTGCACTGCAAGCTGTA
TCACGCCGATACCCCGCTGGCCCTGTCCGACGTGC
TGCCGATCCTGGAAAACCTCGGCCTGCGCGTGCTG
GGTGAGTTCCCGTACCGCCTGCGTCATACCAACGG
CCGCGAGTTCTGGATCCACGACTTCGCGTTCACCG
CTGCCGAAGGCCTGGACCTGGACATCCAGCAACTC
AACGACACCCTGCAGGACGCGTTCGTCCACATCGT
CCGTGGCGATGCCGAAAACGATGCGTTCAACCGTC
TGGTGCTGACCGCCGGCCTGCCATGGCGCGACGTG
GCGCTGCTGCGTGCCTACGCCCGCTACCTGAAGCA
GATCCGCCTGGGCTTCGACCTCGGCTACATCGCCA
GCACCCTGAACAACCACACCGACATCGCTCGCGAA
CTGACCCGGTTGTTCAAGACCCGTTTCTACCTGGC
CCGCAAGCTGGGCAGCGAGGATCTGGACGACAAGC
AACAGCGTCTGGAACAGGCCATCCTGACCGCGCTG
GACGACGTTCAAGTCCTCAACGAAGACCGCATCCT
GCGTCGTTACCTGGACCTGATCAAAGCAACCCTGC
GCACCAACTTCTACCAGACCGACGCCAACGGCCAG
AACAAGTCGTACTTCAGCTTCAAGTTCAACCCGCA
CTTGATTCCTGAACTGCCGAAACCGGTGCCGAAGT
TCGAAATCTTCGTTTACTCGCCACGCGTCGAAGGC
GTGCACCTGCGCTTCGGCAACGTTGCTCGTGGTGG
TCTGCGCTGGTCGGACCGTGAAGAAGACTTCCGTA
CCGAAGTCCTCGGCCTGGTAAAAGCCCAGCAAGTG
AAGAACTCGGTCATCGTGCCGGTGGGGGCGAAGGG
CGGCTTCCTGCCGCGTCGCCTGCCACTGGGCGGCA
GCCGTGACGAGATCGCGGCCGAGGGCATCGCCTGC
TACCGCATCTTCATTTCGGGCCTGTTGGACATCAC
CGACAACCTGAAAGACGGCAAACTGGTACCGCCGG
CCAACGTCGTGCGGCATGACGACGATGACCCGTAC
CTGGTGGTCGCGGCGGACAAGGGCACTGCAACCTT
CTCCGACATCGCCAACGGCATCGCCATCGACTACG
GCTTCTGGCTGGGTGACGCGTTCGCGTCCGGTGGT
TCGGCCGGTTACGACCACAAGAAAATGGGCATCAC
CGCCAAGGGCGCGTGGGTCGGCGTACAGCGCCACT
TCCGCGAGCGCGGCATCAATGTCCAGGAAGACAGC
ATCACGGTGGTCGGCGTGGGCGACATGGCCGGTGA
CGTGTTCGGTAACGGCCTGTTGATGTCTGACAAGC
TGCAACTGGTTGCTGCGTTCAACCACCTGCACATC
TTCATCGACCCGAACCCGAACCCGGCCACCAGCTT
CGCCGAGCGTCAGCGCATGTTCGATCTGCCGCGCT
CGGCCTGGTCCGACTACGACACCAGCATCATGTCC
GAAGGCGGCGGCATCTTCTCGCGCAGCGCGAAGAG
CATCGCCATCTCGCCACAGATGAAAGAGCGCTTCG
ACATCCAGGCCGACAAGCTGACCCCGACCGAACTG
CTGAACGCCTTGCTCAAGGCGCCGGTGGATCTGCT
GTGGAACGGCGGTATCGGTACCTACGTCAAAGCCA
GCACCGAAAGTCACGCCGATGTCGGCGACAAGGCC
AACGATGCGCTGCGCGTGAACGGCAACGAACTGCG
CTGCAAAGTGGTGGGCGAGGGCGGTAACCTCGGCA
TGACCCAATTGGGTCGTGTGGAGTTCGGTCTCAAT
GGCGGCGGTTCCAACACCGACTTCATCGACAACGC
CGGTGGCGTGGACTGCTCCGACCACGAAGTGAACA
TCAAGATCCTGCTGAACGAAGTGGTTCAGGCCGGC
GACATGACCGACAAGCAACGCAACCAGTTGCTGGC
GAGCATGACCGACGAAGTCGGTGGTCTGGTGCTGG
GCAACAACTACAAGCAGACTCAGGCCCTGTCCCTG
GCGGCCCGCCGTGCTTATGCGCGGATCGCCGAGTA
CAAGCGTCTGATGAGCGACCTGGAGGGCCGTGGCA
AGCTGGATCGCGCCATCGAGTTCCTGCCGACCGAA
GAGCAACTGGCCGAACGCGTTGCCGAAGGCCATGG
CCTGACCCGTCCTGAGCTGTCGGTGCTGATCTCGT
ACAGCAAGATCGACCTCAAGGAGCAGCTGCTGGGC
TCCCTGGTGCCGGACGACGACTACCTGACCCGCGA
CATGGAAACGGCGTTCCCGCCGACCCTGGTCAGCA
AGTTCTCCGAAGCTATGCGTCGTCACCGCCTCAAG
CGCGAGATCGTCAGCACCCAGATCGCCAACGATCT
GGTCAACCACATGGGCATCACCTTCGTTCAGCGAC
TCAAAGAGTCCACGGGCATGACCCCGGCGAATGTT
GCCGGTGCGTATGTGATTGTTCGGGATATCTTCCA
CCTCCCGCACTGGTTCCGTCAGATCGAAGCGCTGG
ACTACCAGGTTTCCGCTGACGTGCAGCTGGAGCTG
ATGGACGAGCTGATGCGTCTGGGCCGTCGCGCTAC
GCGCTGGTTCCTGCGTGCCCGTCGCAACGAGCAGA
ACGCTGCCCGTGACGTCGCGCATTTCGGTCCGCAC
CTCAAAGAGCTGGGCCTGAAGCTGGACGAGCTGCT
GAGCGGCGAAATCCGCGAAAACTGGCAAGAGCGTT
ATCAGGCGTACGTCGCCGCCGGTGTTCCGGAACTG
CTGGCGCGTATGGTGGCGGGGACGACCCACCTCTA
CACGCTGCTGCCGATCATCGAAGCGGCCGACGTGA
CCGGCCAGGATCCAGCCGAAGTGGCCAAGGCGTAC
TTCGCCGTGGGCAGCGCGCTGGACATCACCTGGTA
CATCTCGCAGATCAGCGCCTTGCCGGTTGAAAACA
ACTGGCAGGCCCTGGCCCGTGAAGCGTTCCGCGAC
GACGTCGACTGGCAGCAACGCGCGATTACCATCGC
CGTTCTGCAAGCGGGTGGCGGTGATTCGGACGTGG
AAACCCGTCTGGCACTGTGGATGAAGCAGAACGAC
GCCATGATCGAACGCTGGCGCGCCATGCTGGTGGA
AATCCGTGCCGCCAGCGGCACCGACTACGCCATGT
ACGCGGTGGCCAACCGCGAGCTGAACGACGTGGCG
CTGAGCGGTCAGGCAGTTGTGCCTGCTGCGGCGAC
TGCGGAGCTTGAGCTTGCTTGA
SEQ ID NO. 7
MIHAPSRWGVFPSLGLCSPDVVWNEHPSLYMDKEE
TSMTNLQTFELPTEVTGCAADISLGRALIQAWQKD
GIFQIKTDSEQDRKTQEAMAASKQFCKEPLTFKSS
CVSDLTYSGYVASGEEVTAGKPDFPEIFTVCKDLS
VGDQRVKAGWPCHGPVPWPNNTYQKSMKTFMEELG
LAGERLLKLTALGFELPINTFTDLTRDGWHHMRVL
RFPPQTSTLSRGIGAHTDYGLLVIAAQDDVGGLYI
RPPVEGEKRNRNWLPGESSAGMFEHDEPWTFVTPT
PGVWTVFPGDILQFMTGGQLLSTPHKVKLNTRERF
ACAYFHEPNFEASAYPLFEPSANERIHYGEHFTNM
FMRCYPDRITTQRINKENRLAHLEDLKKYSDTRAT
GS
SEQ ID. NO. 8
ATGATACACGCTCCAAGTAGATGGGGAGTATTTCC
CTCACTAGGGTTATGCAGCCCGGACGTTGTGTGGA
ATGAGCATCCGAGCCTGTACATGGACAAAGAGGAA
ACCAGCATGACCAACCTGCAGACCTTTGAACTGCC
GACCGAAGTGACCGGTTGCGCGGCGGACATCAGCC
TGGGTCGTGCGCTGATTCAGGCGTGGCAAAAGGAT
GGTATCTTCCAGATTAAAACCGACAGCGAGCAGGA
TCGTAAGACCCAAGAAGCGATGGCGGCGAGCAAGC
AATTTTGCAAAGAGCCGCTGACCTTCAAAAGCAGC
TGCGTTAGCGACCTGACCTACAGCGGTTATGTGGC
GAGCGGCGAGGAAGTTACCGCGGGCAAGCCGGATT
TCCCGGAAATTTTTACCGTGTGCAAGGACCTGAGC
GTGGGCGATCAGCGTGTTAAAGCGGGTTGGCCGTG
CCATGGTCCGGTTCCGTGGCCGAACAACACCTATC
AAAAGAGCATGAAAACCTTTATGGAGGAACTGGGT
CTGGCGGGCGAGCGTCTGCTGAAACTGACCGCGCT
GGGTTTTGAACTGCCGATCAACACCTTCACCGACC
TGACCCGTGATGGCTGGCACCACATGCGTGTGCTG
CGTTTCCCGCCGCAGACCAGCACCCTGAGCCGTGG
TATTGGTGCGCACACCGACTACGGTCTGCTGGTGA
TTGCGGCGCAAGACGATGTTGGTGGCCTGTATATC
CGTCCGCCGGTGGAGGGCGAAAAGCGTAACCGTAA
CTGGCTGCCGGGCGAGAGCAGCGCGGGCATGTTTG
AGCACGACGAACCGTGGACCTTCGTTACCCCGACC
CCGGGTGTGTGGACCGTTTTTCCGGGCGATATTCT
GCAGTTCATGACCGGTGGCCAACTGCTGAGCACCC
CGCACAAGGTTAAACTGAACACCCGTGAACGTTTC
GCGTGCGCGTACTTTCACGAGCCGAACTTCGAAGC
GAGCGCGTATCCGCTGTTCGAGCCGAGCGCGAACG
AACGTATCCACTACGGCGAGCACTTCACCAACATG
TTTATGCGTTGCTATCCGGATCGTATCACCACCCA
ACGTATTAACAAAGAAAACCGTCTGGCGCACCTGG
AAGACCTGAAGAAATACAGCGACACCCGTGCGACC
GGCAGC
SEQ ID NO. 9
MYKLVQTIVNSDEKNVLGDFILELGKDHKRYFLRN
EILQAFADYCHQFPKPAYFYHSSSLGTFIQYTHEI
ILDGENTWFVVRPKIASQEVWLLSADLTKFELMTP
KALLDVSDRLVKRYQPHILEIDLHPFYSAAPRIDD
SRNIGQGLTVLNHYFCNQALTDPEYWIDALFQSLK
RLEYNGIKLLISNHIHSGLQLTKQIKLALEFVSHL
SPQTPYIKFKFHLQELGLEPGWGNNAARVRETLEL
LERLMDNPEPAILETFVSRICAVFRVVLISIHGWV
AQEDVLGRDETLGQVIYVLEQARSLENKMRAEIKL
AGLDTLGIKPHIIILTRLIPNCEGTFCNLPLEKVD
GTENAWILRVPFAESRPEITNNWISKFEIWPYLEK
FALDAEAELLKQFQGKPNLIIGNYSDGNLVAFILS
RKMKVTQCNIAHSLEKPKYLFSNLYWQDLEAQYHF
SAQFTADLISMNAADFIITSSYQEIVGTPDTMGQY
ESYKCFTMPNLYHVIDGIDLFSPKFNVVLPGVSEN
IFFPYNQTTNRESHRRQHIQDLIFHQEHPEILGKL
DHPHKKPIFSVSPITSIKNLTGLVECFGKSEELQK
HSNLILLTSKLHPDLGTNSEEIQEIAKIHAIIDQY
HLHHKIRWLGMRLPLRDIAETYRVIADFQGIYIHF
ALYESFSRSILEAMISGLPTFTTQFGGSLEIIENH
DQGFNLNPTDLAGTAKTIINFLEKCENYPEHWLEN
SQWMIERIRHKYNWNSHTNQLLLLTKMFSFWNFIY
PEDNEARDRYMESLFHLLYKPIADHILSEHLSKIR
NHN
SEQ ID NO. 10:
ATGTATAAATTAGTGCAAACTATTGTTAACAGTGA
TGAAAAAAATGTTTTAGGTGACTTTATCTTAGAAT
TAGGCAAGGATCATAAACGTTACTTTTTAAGAAAT
GAGATTTTACAAGCTTTTGCAGATTATTGTCACCA
ATTCCCAAAACCCGCTTATTTTTATCACTCTTCCT
CTTTAGGGACATTCATTCAATACACCCATGAAATA
ATTTTAGATGGTGAAAATACTTGGTTTGTAGTTAG
ACCAAAGATTGCGAGTCAAGAAGTATGGTTATTAA
GCGCGGACTTGACTAAGTTTGAGTTAATGACACCG
AAAGCATTATTAGATGTGAGCGATCGCTTAGTAAA
GCGTTATCAACCGCACATTTTAGAAATTGATCTCC
ATCCCTTTTATTCAGCAGCACCAAGAATTGATGAT
TCCAGAAATATTGGCCAAGGTTTAACCGTTCTTAA
TCATTATTTTTGTAATCAAGCATTGACAGATCCTG
AATATTGGATTGACGCATTATTTCAATCATTAAAA
AGATTAGAATATAACGGCATCAAATTATTAATTAG
TAATCATATTCATTCAGGTTTGCAACTAACAAAGC
AAATCAAACTAGCGTTAGAATTTGTGAGTCATTTA
TCCCCCCAGACACCATATATAAAATTTAAATTTCA
TCTTCAAGAACTCGGTTTAGAACCAGGTTGGGGTA
ATAATGCAGCCAGAGTCAGAGAAACCTTAGAACTG
CTGGAAAGACTCATGGATAATCCCGAACCTGCAAT
TTTAGAAACCTTTGTTTCTCGCATTTGTGCAGTTT
TCCGCGTCGTCCTTATTTCCATCCATGGTTGGGTT
GCACAAGAAGATGTTTTAGGCAGAGATGAAACATT
AGGACAAGTTATTTATGTTTTAGAACAAGCCCGCA
GTTTAGAAAATAAAATGCGGGCAGAAATTAAACTT
GCAGGTTTAGATACATTAGGAATTAAACCCCATAT
CATTATATTAACTCGACTGATTCCCAATTGTGAAG
GCACATTTTGTAACTTACCATTAGAAAAAGTTGAT
GGTACAGAAAATGCTTGGATTTTGCGCGTTCCTTT
TGCAGAATCTCGACCGGAAATTACCAACAACTGGA
TTTCTAAATTTGAAATTTGGCCTTATTTAGAAAAA
TTTGCTCTTGATGCCGAAGCAGAACTTTTAAAACA
ATTCCAAGGAAAGCCCAATCTAATTATTGGTAACT
ACAGTGACGGGAACTTAGTTGCTTTTATTCTCTCC
CGAAAAATGAAAGTTACCCAATGTAATATTGCCCA
TTCCCTCGAAAAACCTAAATATCTATTTAGTAACT
TATATTGGCAAGATTTAGAAGCACAATATCACTTT
TCTGCCCAATTTACCGCTGATTTAATCAGTATGAA
TGCCGCAGATTTTATTATCACATCATCCTATCAAG
AAATTGTAGGTACACCAGATACAATGGGACAATAT
GAATCTTATAAATGTTTCACCATGCCCAACTTATA
TCATGTAATTGATGGCATTGATTTATTTAGCCCTA
AATTCAATGTGGTATTACCAGGAGTCAGTGAAAAT
ATATTTTTTCCCTACAACCAAACAACAAATAGAGA
ATCCCACCGTCGTCAACATATCCAAGACCTAATTT
TCCATCAAGAACACCCAGAAATTCTCGGTAAATTA
GATCATCCTCATAAAAAACCGATCTTTTCCGTTAG
TCCCATTACCTCAATTAAAAACCTCACAGGTTTAG
TTGAATGTTTCGGTAAAAGTGAAGAATTACAAAAA
CATAGTAACCTAATTTTATTAACCAGTAAACTTCA
TCCAGACTTAGGAACAAACTCCGAAGAAATTCAAG
AAATAGCAAAAATTCATGCGATTATTGATCAATAT
CATCTTCACCATAAAATCCGCTGGTTGGGAATGCG
TCTTCCTCTCCGCGATATTGCTGAAACCTATCGTG
TAATTGCCGATTTTCAAGGGATTTATATTCACTTT
GCCCTTTATGAATCCTTTAGCAGAAGTATTTTAGA
AGCAATGATTTCTGGATTACCAACTTTTACAACTC
AATTTGGTGGTTCATTAGAAATTATTGAAAACCAT
GATCAAGGATTTAACCTCAACCCCACAGACTTAGC
AGGAACAGCCAAAACAATTATCAACTTCTTAGAAA
AATGTGAAAATTATCCAGAACATTGGCTAGAAAAT
TCTCAATGGATGATTGAACGCATTCGCCATAAATA
TAACTGGAATTCCCACACAAATCAACTCCTGTTAT
TAACGAAAATGTTTAGCTTTTGGAACTTCATCTAT
CCCGAAGATAACGAAGCCAGAGATCGTTACATGGA
AAGTTTATTTCATCTTCTTTATAAACCTATAGCTG
ACCATATTTTATCAGAACATCTAAGCAAAATCAGA
AATCATAATTAA
SEQ ID NO. 11:
MAAQNLYILHIQTHGLLRGQNLELGRDADTGGQTK
YVLELAQAQAKSPQVQQVDIITRQITDPRVSVGYS
QAIEPFAPKGRIVRLPFGPKRYLRKELLWPHLYTF
ADAILQYLAQQKRTPTWIQAHYADAGQVGSLLSRW
LNVPLIFTGHSLGRIKLKKLLEQDWPLEEIEAQFN
IQQRIDAEEMTLTHADWIVASTQQEVEEQYRVYDR
YNPERKLVIPPGVDTDRFRFQPLGDRGVVLQQELS
RFLRDPEKPQILCLCRPAPRKNVPALVRAFGEHPW
LRKKANLVLVLGSRQDINQMDRGSRQVFQEIFHLV
DRYDLYGSVAYPKQHQADDVPEFYRLAAHSGGVFV
NPALTEPFGLTILEAGSCGVPVVATHDGGPQEILK
HCDFGTLVDVSRPANIATALATLLSDRDLWQCYHR
NGIEKVPAHYSWDQHVNTLFERMETVALPRRRAVS
FVRSRKRLIDAKRLVVSDIDNTLLGDRQGLENLMT
YLDQYRDHFAFGIATGRRLDSAQEVLKEWGVPSPN
FWVTSVGSEIHYGTDAEPDISWEKHINRNWNPQRI
RAVMAQLPFLELQPEEDQTPFKVSFFVRDRHETVL
REVRQHLRRHRLRLKSIYSHQEFLDILPLAASKGD
AIRHLSLRWRIPLENILVAGDSGNDEEMLKGHNLG
VVVGNYSPELEPLRSYERVYFAEGHYANGILEALK
HYRFFEAIA
SEQ ID NO. 12:
GTGGCAGCTCAAAATCTCTACATTCTGCACATTCA
GACCCATGGTCTGCTGCGAGGGCAGAACTTGGAAC
TGGGGCGAGATGCCGACACCGGCGGGCAGACCAAG
TACGTCTTAGAACTGGCTCAAGCCCAAGCTAAATC
CCCACAAGTCCAACAAGTCGACATCATCACCCGCC
AAATCACCGACCCCCGCGTCAGTGTTGGTTACAGT
CAGGCGATCGAACCCTTTGCGCCCAAAGGTCGGAT
TGTCCGTTTGCCTTTTGGCCCCAAACGCTACCTCC
GTAAAGAGCTGCTTTGGCCCCATCTCTACACCTTT
GCGGATGCAATTCTCCAATATCTGGCTCAGCAAAA
GCGCACCCCGACTTGGATTCAGGCCCACTATGCTG
ATGCTGGCCAAGTGGGATCACTGCTGAGTCGCTGG
TTGAATGTACCGCTAATTTTCACAGGGCATTCTCT
GGGGCGGATCAAGCTAAAAAAGCTGTTGGAGCAAG
ACTGGCCGCTTGAGGAAATTGAAGCGCAATTCAAT
ATTCAACAGCGAATTGATGCGGAGGAGATGACGCT
CACTCATGCTGACTGGATTGTCGCCAGCACTCAGC
AGGAAGTGGAGGAGCAATACCGCGTTTACGATCGC
TACAACCCAGAGCGCAAGCTTGTCATTCCACCGGG
TGTCGATACCGATCGCTTCAGGTTTCAGCCCTTGG
GCGATCGCGGTGTTGTTCTCCAACAGGAACTGAGC
CGCTTTCTGCGCGACCCAGAAAAACCTCAAATTCT
CTGCCTCTGTCGCCCCGCACCTCGCAAAAATGTAC
CGGCGCTGGTGCGAGCCTTTGGCGAACATCCTTGG
CTGCGCAAAAAAGCCAACCTTGTCTTAGTACTGGG
CAGCCGCCAAGACATCAACCAGATGGATCGCGGCA
GTCGGCAGGTGTTCCAAGAGATTTTCCATCTGGTC
GATCGCTACGACCTCTACGGCAGCGTCGCCTATCC
CAAACAGCATCAGGCTGATGATGTGCCGGAGTTCT
ATCGCCTAGCGGCTCATTCCGGCGGGGTATTCGTC
AATCCGGCGCTGACCGAACCTTTTGGTTTGACAAT
TTTGGAGGCAGGAAGCTGCGGCGTGCCGGTGGTGG
CAACCCATGATGGCGGCCCCCAGGAAATTCTCAAA
CACTGTGATTTCGGCACTTTAGTTGATGTCAGCCG
ACCCGCTAATATCGCGACTGCACTCGCCACCCTGC
TGAGCGATCGCGATCTTTGGCAGTGCTATCACCGC
AATGGCATTGAAAAAGTTCCCGCCCATTACAGCTG
GGATCAACATGTCAATACCCTGTTTGAGCGCATGG
AAACGGTGGCTTTGCCTCGTCGTCGTGCTGTCAGT
TTCGTACGGAGTCGCAAACGCTTGATTGATGCCAA
ACGCCTTGTCGTTAGTGACATCGACAACACACTGT
TGGGCGATCGTCAAGGACTCGAGAATTTAATGACC
TATCTCGATCAGTATCGCGATCATTTTGCCTTTGG
AATTGCCACGGGGCGTCGCCTAGACTCTGCCCAAG
AAGTCTTGAAAGAGTGGGGCGTTCCTTCGCCAAAC
TTCTGGGTGACTTCCGTCGGCAGCGAGATTCACTA
TGGCACCGATGCTGAACCGGATATCAGCTGGGAAA
AGCATATCAATCGCAACTGGAATCCTCAGCGAATT
CGGGCAGTAATGGCACAACTACCCTTTCTTGAACT
GCAGCCGGAAGAGGATCAAACACCCTTCAAAGTCA
GCTTCTTTGTCCGCGATCGCCACGAGACTGTGCTG
CGAGAAGTACGGCAACATCTTCGCCGCCATCGCCT
GCGGCTGAAGTCAATCTATTCCCATCAGGAGTTTC
TTGACATTCTGCCGCTAGCTGCCTCGAAAGGGGAT
GCGATTCGCCACCTCTCACTCCGCTGGCGGATTCC
TCTTGAGAACATTTTGGTGGCAGGCGATTCTGGTA
ACGATGAGGAAATGCTCAAGGGCCATAATCTCGGC
GTTGTAGTTGGCAATTACTCACCGGAATTGGAGCC
ACTGCGCAGCTACGAGCGCGTCTATTTTGCTGAGG
GCCACTATGCTAATGGCATTCTGGAAGCCTTAAAA
CACTATCGCTTTTTTGAGGCGATCGCTTAA
SEQ ID NO. 13:
CAATTGCCCTAAGACAGTTGTCGTCTTTCGAAGTC
TAGTTAACATTAGGGGCGATTCTTTGTTTCCACTG
AGTGGAAGCAAACGGTATCAAGGTTGCAGGCAGAC
TCAAGGTCTAGATTGCTTCACAGCTTGTGTGGCTA
TATTTATTATCTTCATTATTGATGGTAGTTGTGGG
TGGATTTAAGATGGAAAAGTAACAGATAAATGTCG
TCTTTAAGGGCGATCTAGATCGTATCGTTTTTAAT
TCCTAGGTCGGCATTTATTAATCAACCTCGATACA
ATATTTTTTTGTAAAAACTTCTAGATAAATGACTC
AAGTCTCATTGAAAGTCTGGGGTGTTGCCTCCCCA
GTCAATTCAAGATTACCAAGGCCTCGCATCGCCTC
TTCTATTTTGTTTGAAGGGGACCTAACGTGTTGCG
CCAAGCTAGTTCTCGACAGAGCATCTCAAGAGCGC
GTTGCTCGCGGGGGGCAAACAGTTGGAGATCAGCC
AGCTCTTGCAAGACTTGTTGGGTGAGGTGGCTGGC
AAAGCTACCGGCATAGCGCAGTAAGAGACTGTAGT
AGCGAAATTGCGGTTGGCCGTTGCAATCGCGGTGA
AAGGCAGCAATTTCTGCTTCGCTGAGGCAGTAGAC
AGGGGTATTGACCGGCACGATCGCGCGAGGAATGC
CCTGTTCGCCGTAGCGATCGCCGCCCTCTGTTTCC
GCTAAGCTGCCGATCAAAACCGTGGAGAGCAGCGT
GCGGGTTTCATTGATTAAATCAGGCGTGAATAGTG
GGTCGGGCCCACTTGAAAGACGCGGCCCGTTGTTC
AGAAAGAGGGGATTAAACAACTGCGAGTTGTAGAC
CACTCCGATCGCCCAATGGCGATCGCCTTCCAAAC
GAACAAAGCTGCCAAATCCATAGCTCTCGGCGGCT
GGTGGATTGGAGACATCCATGTCGTCATCCACTTG
GACAACGTAGTCACAGTGCGAGTTGGATTTGACAA
CTTTGCCGAGGCGCATGGTGCTGCCAGTGTTACAA
CCAATTAACCAATTCTGACATATGGACACCATCGA
ATGGTGCAAAACCTTTCGCGGTATGGCATGATAGC
GCCCGGAAGAGAGTCAATTCAGGGTGGTGAATGTG
AAACCAGTAACGTTATACGATGTCGCAGAGTATGC
CGGTGTCTCTTATCAGACCGTTTCCCGCGTGGTGA
ACCAGGCCAGCCACGTTTCTGCGAAAACGCGGGAA
AAAGTGGAAGCGGCGATGGCGGAGCTGAATTACAT
TCCCAACCGCGTGGCACAACAACTGGCGGGCAAAC
AGTCGTTGCTGATTGGCGTTGCCACCTCCAGTCTG
GCCCTGCACGCGCCGTCGCAAATTGTCGCGGCGAT
TAAATCTCGCGCCGATCAACTGGGTGCCAGCGTGG
TGGTGTCGATGGTAGAACGAAGCGGCGTCGAAGCC
TGTAAAGCGGCGGTGCACAATCTTCTCGCGCAACG
CGTCAGTGGGCTGATCATTAACTATCCGCTGGATG
ACCAGGATGCCATTGCTGTGGAAGCTGCCTGCACT
AATGTTCCGGCGTTATTTCTTGATGTCTCTGACCA
GACACCCATCAACAGTATTATTTTCTCCCATGAAG
ACGGTACGCGACTGGGCGTGGAGCATCTGGTCGCA
TTGGGTCACCAGCAAATCGCGCTGTTAGCGGGCCC
ATTAAGTTCTGTCTCGGCGCGTCTGCGTCTGGCTG
GCTGGCATAAATATCTCACTCGCAATCAAATTCAG
CCGATAGCGGAACGGGAAGGCGACTGGAGTGCCAT
GTCCGGTTTTCAACAAACCATGCAAATGCTGAATG
AGGGCATCGTTCCCACTGCGATGCTGGTTGCCAAC
GATCAGATGGCGCTGGGCGCAATGCGCGCCATTAC
CGAGTCCGGGCTGCGCGTTGGTGCGGATATCTCGG
TAGTGGGATACGACGATACCGAAGACAGCTCATGT
TATATCCCGCCGTTAACCACCATCAAACAGGATTT
TCGCCTGCTGGGGCAAACCAGCGTGGACCGCTTGC
TGCAACTCTCTCAGGGCCAGGCGGTGAAGGGCAAT
CAGCTGTTGCCCGTTTCACTGGTGAAAAGAAAAAC
CACCCTGGCGCCCAATACGCAAACCGCCTCTCCCC
GCGCGTTGGCCGATTCATTAATGCAGCTGGCACGA
CAGGTTTCCCGACTGGAAAGCGGGCAGTGAGCTTT
GTTGACAATTAATCATCCGGCTCGTATAATGTGTG
GAATTGTGAGCGGATAACAAGAAGGAGATGAGTAT
TCAACATTTCCGTGTCGCCCTTATTCCCTTTTTTG
CGGCATTTTGCCTTCCTGTTTTTGCTCACCCAGAA
ACGCTGGTGAAAGTAAAAGATGCTGAAGATCAGTT
GGGTGCACGAGTGGGTTACATCGAACTGGATCTCA
ACAGCGGTAAGATCCTTGAGAGTTTTCGCCCCGAA
GAACGTTTTCCAATGATGAGCACTTTTAAAGTTCT
GCTATGTGGCGCGGTATTATCCCGTATTGACGCCG
GGCAAGAGCAACTCGGTCGCCGCATACACTATTCT
CAGAATGACTTGGTTGAGTACTCACCAGTCACAGA
AAAGCATCTTACGGATGGCATGACAGTAAGAGAAT
TATGCAGTGCTGCCATAACCATGAGTGATAACACT
GCGGCCAACTTACTTCTGACAACGATCGGAGGACC
GAAGGAGCTAACCGCTTTTTTGCACAACATGGGGG
ATCATGTAACTCGCCTTGATCGTTGGGAACCGGAG
CTGAATGAAGCCATACCAAACGACGAGCGTGACAC
CACGATGCCTGTAGCAATGGCAACAACGTTGCGCA
AACTATTAACTGGCGAACTACTTACTCTAGCTTCC
CGGCAACAATTAATAGACTGGATGGAGGCGGATAA
AGTTGCAGGACCACTTCTGCGCTCGGCCCTTCCGG
CTGGCTGGTTTATTGCTGATAAATCTGGAGCCGGT
GAGCGTGGGTCTCGCGGTATCATTGCAGCACTGGG
GCCAGATGGTAAGCCCTCCCGTATCGTAGTTATCT
ACACGACGGGGAGTCAGGCAACTATGGATGAACGA
AATAGACAGATCGCTGAGATAGGTGCCTCACTGAT
TAAGCATTGGTAATTGTTCAGAACGCTCGGTCTTG
CACACCGGGCGTTTTTTCTTTGTGAGTCCAGGTAC
CAATCAATCTCCCCCAAGTCAAGCGGCGCTGAGAC
CCAGTGTCTGCCGGTGAGTCAGTCTTGGCAAGCAA
ACTGTGCCTTTGCGATTTCTTACCCTACGCAGCTC
CGGGATCGATCGGAGGTAACCAAGGCTACGGACAA
TGGCGCGGGCACCAGCTGTTGGTAAACTGAGGAGC
GATCGCCGCTTCAGTCCAAAGGCTATGACGCAAAA
ATCCGTTGTTATTGCTCCGTCCATTCTGTCAGCGG
ATTTCAGCCGCTTGGGCGACGATGTCCGCGCTGTT
GACCAGGCTGGCGCTGACTGGATTCACGTCGATGT
GATGGATGGTCGCTTCGTCCCTAACATCACCATTG
GACCGCTGATCGTTGAAGCGCTGCGCCCGGTGACC
CAAAAGCCGTTGGACGTCCACTTGATGATCGTCGA
GCCGGAAAAATATGTGCCGGATTTCGCGAAAGCAG
GGGCTGACATCATCTCGGTCCAAGCAGAAGCTTGC
CCCCACCTGCACCGCAACTTGGCTCAGATCAAAGA
CCTCGGCAAGCAAGCAGGCGTCGTCCTCAACCCCT
CTACCCCAGTCGAAACCCTGGAATACGTGCTGGAG
TTGTGCGACCTGATTTTGATCATGAGCGTCAACCC
TGGCTTCGGTGGTCAGAGTTTCATCCCAGCTGTCC
TGCCGAAAATCCGTAAGCTGCGCGCCATGTGCGAT
GAGCGTGGCCTTGATCCTTGGATTGAAGTCGATGG
CGGCTTGAAAGCCAATAACACTTGGCAGGTGCTGG
AAGCCGGTGCTAACGCAATTGTGGCGGGCTCGGCA
GTCTTCAACGCGCCGGACTATGCTGAAGCGATCGC
GGCGATTCGCAACAGCAAGCGTCCTGAACTTGTCA
CTGCTTAGGCTTCTCGCTCAACGCTCAGTGGAGCA
ATCTGAATCTTGCAGCCCTTCAGTGGATCAGTCTG
CTGAGGGGTTTTGCTTTAGGATGGGCGATCGCGAG
TAGGGACACGGATCGCTGGTA
SEQ ID NO. 14:
ATGGACGCTTACTCTACCCGTCCGCTGACCCTGTC
TCACGGTTCTCTGGAACACGTTCTGCTGGTTCCGA
CCGCTTCTTTCTTCATCGCTTCTCAGCTGCAGGAA
CAGTTCAACAAAATCCTGCCGGAACCGACCGAAGG
TTTCGCTGCTGACGACGAACCGACCACCCCGGCTG
AACTGGTTGGTAAATTCCTGGGTTACGTTTCTTCT
CTGGTTGAACCGTCTAAAGTTGGTCAGTTCGACCA
GGTTCTGAACCTGTGCCTGACCGAATTCGAAAACT
GCTACCTGGAAGGTAACGACATCCACGCTCTGGCT
GCTAAACTGCTGCAGGAAAACGACACCACCCTGGT
TAAAACCAAAGAACTGATCAAAAACTACATCACCG
CTCGTATCATGGCTAAACGTCCGTTCGACAAAAAA
TCTAACTCTGCTCTGTTCCGTGCTGTTGGTGAAGG
TAACGCTCAGCTGGTTGCTATCTTCGGTGGTCAGG
GTAACACCGACGACTACTTCGAAGAACTGCGTGAC
CTGTACCAGACCTACCACGTTCTGGTTGGTGACCT
GATCAAATTCTCTGCTGAAACCCTGTCTGAACTGA
TCCGTACCACCCTGGACGCTGAAAAAGTTTTCACC
CAGGGTCTGAACATCCTGGAATGGCTGGAAAACCC
GTCTAACACCCCGGACAAAGACTACCTGCTGTCTA
TCCCGATCTCTTGCCCGCTGATCGGTGTTATCCAG
CTGGCTCACTACGTTGTTACCGCTAAACTGCTGGG
TTTCACCCCGGGTGAACTGCGTTCTTACCTGAAAG
GTGCTACCGGTCACTCTCAGGGTCTGGTTACCGCT
GTTGCTATCGCTGAAACCGACTCTTGGGAATCTTT
CTTCGTTTCTGTTCGTAAAGCTATCACCGTTCTGT
TCTTCATCGGTGTTCGTTGCTACGAAGCTTACCCG
AACACCTCTCTGCCGCCGTCTATCCTGGAAGACTC
TCTGGAAAACAACGAAGGTGTTCCGTCTCCGATGC
TGTCTATCTCTAACCTGACCCAGGAACAGGTTCAG
GACTACGTTAACAAAACCAACTCTCACCTGCCGGC
TGGTAAACAGGTTGAAATCTCTCTGGTTAACGGTG
CTAAAAACCTGGTTGTTTCTGGTCCGCCGCAGTCT
CTGTACGGTCTGAACCTGACCCTGCGTAAAGCTAA
AGCTCCGTCTGGTCTGGACCAGTCTCGTATCCCGT
TCTCTGAACGTAAACTGAAATTCTCTAACCGTTTC
CTGCCGGTTGCTTCTCCGTTCCACTCTCACCTGCT
GGTTCCGGCTTCTGACCTGATCAACAAAGACCTGG
TTAAAAACAACGTTTCTTTCAACGCTAAAGACATC
CAGATCCCGGTTTACGACACCTTCGACGGTTCTGA
CCTGCGTGTTCTGTCTGGTTCTATCTCTGAACGTA
TCGTTGACTGCATCATCCGTCTGCCGGTTAAATGG
GAAACCACCACCCAGTTCAAAGCTACCCACATCCT
GGACTTCGGTCCGGGTGGTGCTTCTGGTCTGGGTG
TTCTGACCCACCGTAACAAAGACGGTACCGGTGTT
CGTGTTATCGTTGCTGGTACCCTGGACATCAACCC
GGACGACGACTACGGTTTCAAACAGGAAATCTTCG
ACGTTACCTCTAACGGTCTGAAAAAAAACCCGAAC
TGGCTGGAAGAATACCACCCGAAACTGATCAAAAA
CAAATCTGGTAAAATCTTCGTTGAAACCAAATTCT
CTAAACTGATCGGTCGTCCGCCGCTGCTGGTTCCG
GGTATGACCCCGTGCACCGTTTCTCCGGACTTCGT
TGCTGCTACCACCAACGCTGGTTACACCATCGAAC
TGGCTGGTGGTGGTTACTTCTCTGCTGCTGGTATG
ACCGCTGCTATCGACTCTGTTGTTTCTCAGATCGA
AAAAGGTTCTACCTTCGGTATCAACCTGATCTACG
TTAACCCGTTCATGCTGCAGTGGGGTATCCCGCTG
ATCAAAGAACTGCGTTCTAAAGGTTACCCGATCCA
GTTCCTGACCATCGGTGCTGGTGTTCCGTCTCTGG
AAGTTGCTTCTGAATACATCGAAACCCTGGGTCTG
AAATACCTGGGTCTGAAACCGGGTTCTATCGACGC
TATCTCTCAGGTTATCAACATCGCTAAAGCTCACC
CGAACTTCCCGATCGCTCTGCAGTGGACCGGTGGT
CGTGGTGGTGGTCACCACTCTTTCGAAGACGCTCA
CACCCCGATGCTGCAGATGTACTCTAAAATCCGTC
GTCACCCGAACATCATGCTGATCTTCGGTTCTGGT
TTCGGTTCTGCTGACGACACCTACCCGTACCTGAC
CGGTGAATGGTCTACCAAATTCGACTACCCGCCGA
TGCCGTTCGACGGTTTCCTGTTCGGTTCTCGTGTT
ATGATCGCTAAAGAAGTTAAAACCTCTCCGGACGC
TAAAAAATGCATCGCTGCTTGCACCGGTGTTCCGG
ACGACAAATGGGAACAGACCTACAAAAAACCGACC
GGTGGTATCGTTACCGTTCGTTCTGAAATGGGTGA
ACCGATCCACAAAATCGCTACCCGTGGTGTTATGC
TGTGGAAAGAATTCGACGAAACCATCTTCAACCTG
CCGAAAAACAAACTGGTTCCGACCCTGGAAGCTAA
ACGTGACTACATCATCTCTCGTCTGAACGCTGACT
TCCAGAAACCGTGGTTCGCTACCGTTAACGGTCAG
GCTCGTGACCTGGCTACCATGACCTACGAAGAAGT
TGCTAAACGTCTGGTTGAACTGATGTTCATCCGTT
CTACCAACTCTTGGTTCGACGTTACCTGGCGTACC
TTCACCGGTGACTTCCTGCGTCGTGTTGAAGAACG
TTTCACCAAATCTAAAACCCTGTCTCTGATCCAGT
CTTACTCTCTGCTGGACAAACCGGACGAAGCTATC
GAAAAAGTTTTCAACGCTTACCCGGCTGCTCGTGA
ACAGTTCCTGAACGCTCAGGACATCGACCACTTCC
TGTCTATGTGCCAGAACCCGATGCAGAAACCGGTT
CCGTTCGTTCCGGTTCTGGACCGTCGTTTCGAAAT
CTTCTTCAAAAAAGACTCTCTGTGGCAGTCTGAAC
ACCTGGAAGCTGTTGTTGACCAGGACGTTCAGCGT
ACCTGCATCCTGCACGGTCCGGTTGCTGCTCAGTT
CACCAAAGTTATCGACGAACCGATCAAATCTATCA
TGGACGGTATCCACGACGGTCACATCAAAAAACTG
CTGCACCAGTACTACGGTGACGACGAATCTAAAAT
CCCGGCTGTTGAATACTTCGGTGGTGAATCTCCGG
TTGACGTTCAGTCTCAGGTTGACTCTTCTTCTGTT
TCTGAAGACTCTGCTGTTTTCAAAGCTACCTCTTC
TACCGACGAAGAATCTTGGTTCAAAGCTCTGGCTG
GTTCTGAAATCAACTGGCGTCACGCTTCTTTCCTG
TGCTCTTTCATCACCCAGGACAAAATGTTCGTTTC
TAACCCGATCCGTAAAGTTTTCAAACCGTCTCAGG
GTATGGTTGTTGAAATCTCTAACGGTAACACCTCT
TCTAAAACCGTTGTTACCCTGTCTGAACCGGTTCA
GGGTGAACTGAAACCGACCGTTATCCTGAAACTGC
TGAAAGAAAACATCATCCAGATGGAAATGATCGAA
AACCGTACCATGGACGGTAAACCGGTTTCTCTGCC
GCTGCTGTACAACTTCAACCCGGACAACGGTTTCG
CTCCGATCTCTGAAGTTATGGAAGACCGTAACCAG
CGTATCAAAGAAATGTACTGGAAACTGTGGATCGA
CGAACCGTTCAACCTGGACTTCGACCCGCGTGACG
TTATCAAAGGTAAAGACTTCGAAATCACCGCTAAA
GAAGTTTACGACTTCACCCACGCTGTTGGTAACAA
CTGCGAAGACTTCGTTTCTCGTCCGGACCGTACCA
TGCTGGCTCCGATGGACTTCGCTATCGTTGTTGGT
TGGCGTGCTATCATCAAAGCTATCTTCCCGAACAC
CGTTGACGGTGACCTGCTGAAACTGGTTCACCTGT
CTAACGGTTACAAAATGATCCCGGGTGCTAAACCG
CTGCAGGTTGGTGACGTTGTTTCTACCACCGCTGT
TATCGAATCTGTTGTTAACCAGCCGACCGGTAAAA
TCGTTGACGTTGTTGGTACCCTGTCTCGTAACGGT
AAACCGGTTATGGAAGTTACCTCTTCTTTCTTCTA
CCGTGGTAACTACACCGACTTCGAAAACACCTTCC
AGAAAACCGTTGAACCGGTTTACCAGATGCACATC
AAAACCTCTAAAGACATCGCTGTTCTGCGTTCTAA
AGAATGGTTCCAGCTGGACGACGAAGACTTCGACC
TGCTGAACAAAACCCTGACCTTCGAAACCGAAACC
GAAGTTACCTTCAAAAACGCTAACATCTTCTCTTC
TGTTAAATGCTTCGGTCCGATCAAAGTTGAACTGC
CGACCAAAGAAACCGTTGAAATCGGTATCGTTGAC
TACGAAGCTGGTGCTTCTCACGGTAACCCGGTTGT
TGACTTCCTGAAACGTAACGGTTCTACCCTGGAAC
AGAAAGTTAACCTGGAAAACCCGATCCCGATCGCT
GTTCTGGACTCTTACACCCCGTCTACCAACGAACC
GTACGCTCGTGTTTCTGGTGACCTGAACCCGATCC
ACGTTTCTCGTCACTTCGCTTCTTACGCTAACCTG
CCGGGTACCATCACCCACGGTATGTTCTCTTCTGC
TTCTGTTCGTGCTCTGATCGAAAACTGGGCTGCTG
ACTCTGTTTCTTCTCGTGTTCGTGGTTACACCTGC
CAGTTCGTTGACATGGTTCTGCCGAACACCGCTCT
GAAAACCTCTATCCAGCACGTTGGTATGATCAACG
GTCGTAAACTGATCAAATTCGAAACCCGTAACGAA
GACGACGTTGTTGTTCTGACCGGTGAAGCTGAAAT
CGAACAGCCGGTTACCACCTTCGTTTTCACCGGTC
AGGGTTCTCAGGAACAGGGTATGGGTATGGACCTG
TACAAAACCTCTAAAGCTGCTCAGGACGTTTGGAA
CCGTGCTGACAACCACTTCAAAGACACCTACGGTT
TCTCTATCCTGGACATCGTTATCAACAACCCGGTT
AACCTGACCATCCACTTCGGTGGTGAAAAAGGTAA
ACGTATCCGTGAAAACTACTCTGCTATGATCTTCG
AAACCATCGTTGACGGTAAACTGAAAACCGAAAAA
ATCTTCAAAGAAATCAACGAACACTCTACCTCTTA
CACCTTCCGTTCTGAAAAAGGTCTGCTGTCTGCTA
CCCAGTTCACCCAGCCGGCTCTGACCCTGATGGAA
AAAGCTGCTTTCGAAGACCTGAAATCTAAAGGTCT
GATCCCGGCTGACGCTACCTTCGCTGGTCACTCTC
TGGGTGAATACGCTGCTCTGGCTTCTCTGGCTGAC
GTTATGTCTATCGAATCTCTGGTTGAAGTTGTTTT
CTACCGTGGTATGACCATGCAGGTTGCTGTTCCGC
GTGACGAACTGGGTCGTTCTAACTACGGTATGATC
GCTATCAACCCGGGTCGTGTTGCTGCTTCTTTCTC
TCAGGAAGCTCTGCAGTACGTTGTTGAACGTGTTG
GTAAACGTACCGGTTGGCTGGTTGAAATCGTTAAC
TACAACGTTGAAAACCAGCAGTACGTTGCTGCTGG
TGACCTGCGTGCTCTGGACACCGTTACCAACGTTC
TGAACTTCATCAAACTGCAGAAAATCGACATCATC
GAACTGCAGAAATCTCTGTCTCTGGAAGAAGTTGA
AGGTCACCTGTTCGAAATCATCGACGAAGCTTCTA
AAAAATCTGCTGTTAAACCGCGTCCGCTGAAACTG
GAACGTGGTTTCGCTTGCATCCCGCTGGTTGGTAT
CTCTGTTCCGTTCCACTCTACCTACCTGATGAACG
GTGTTAAACCGTTCAAATCTTTCCTGAAAAAAAAC
ATCATCAAAGAAAACGTTAAAGTTGCTCGTCTGGC
TGGTAAATACATCCCGAACCTGACCGCTAAACCGT
TCCAGGTTACCAAAGAATACTTCCAGGACGTTTAC
GACCTGACCGGTTCTGAACCGATCAAAGAAATCAT
CGACAACTGGGAAAAATACGAACAGTCTTAA
SEQ ID NO. 15:
AGCTCTACTCCCATTATTATCGTTTTCTGTGATCT
TTTCTACATACTGTGCTTCAAAAGAAAAAGGAAAA
TGCAGCGTGCGTTCCTTCGTCGTCTCAGCAAGCGT
GCCGTCGTTCCTGCAACTGCGGCGCTGCTTCGGTT
TTCTCAGACGCAGTGCGCTGGTCGTGCCCCTGTTA
CCGCGTGCGCAGGCGCTGTTCTTGCCTACCAGTGC
TCTATCCGAGCCTACTCCGATGCCCACCACGAGGA
GAGCGCTACTCGCAGCGGCCAATACCTCCTCGACA
AGAACGACGTGCTGACGCGTGTGCTCGAGGTAGTG
AAGAACTTCGAGAAGGTTGATGCCTCTAAGGTGAC
GCCTGAGTCTCACTTCGTGAACGATCTCGGCCTCA
ACTCTCTCGACGTTGTGGAGGTCGTTTTTGCCATC
GAGCAGGAGTTCATCTTAGATATCCCTGATCACGA
TGCCGAAAAGATCCAGTCCATTCCTGATGCTGTGG
AGTACATTGCGCAGAATCCAATGGCCAAGTAA
SEQ ID NO. 16:
ATGGAAGGTTCTCCGGTTACCTCTGTTCGTCTGTC
TTCTGTTGTTCCGGCTTCTGTTGTTGGTGAAAACA
AACCGCGTCAGCTGACCCCGATGGACCTGGCTATG
AAACTGCACTACGTTCGTGCTGTTTACTTCTTCAA
AGGTGCTCGTGACTTCACCGTTGCTGACGTTAAAA
ACACCATGTTCACCCTGCAGTCTCTGCTGCAGTCT
TACCACCACGTTTCTGGTCGTATCCGTATGTCTGA
CAACGACAACGACACCTCTGCTGCTGCTATCCCGT
ACATCCGTTGCAACGACTCTGGTATCCGTGTTGTT
GAAGCTAACGTTGAAGAATTCACCGTTGAAAAATG
GCTGGAACTGGACGACCGTTCTATCGACCACCGTT
TCCTGGTTTACGACCACGTTCTGGGTCCGGACCTG
ACCTTCTCTCCGCTGGTTTTCCTGCAGATCACCCA
GTTCAAATGCGGTGGTCTGTGCATCGGTCTGTCTT
GGGCTCACATCCTGGGTGACGTTTTCTCTGCTTCT
ACCTTCATGAAAACCCTGGGTCAGCTGGTTTCTGG
TCACGCTCCGACCAAACCGGTTTACCCGAAAACCC
CGGAACTGACCTCTCACGCTCGTAACGACGGTGAA
GCTATCTCTATCGAAAAAATCGACTCTGTTGGTGA
ATACTGGCTGCTGACCAACAAATGCAAAATGGGTC
GTCACATCTTCAACTTCTCTCTGAACCACATCGAC
TCTCTGATGGCTAAATACACCACCCGTGACCAGCC
GTTCTCTGAAGTTGACATCCTGTACGCTCTGATCT
GGAAATCTCTGCTGAACATCCGTGGTGAAACCAAC
ACCAACGTTATCACCATCTGCGACCGTAAAAAATC
TTCTACCTGCTGGAACGAAGACCTGGTTATCTCTG
TTGTTGAAAAAAACGACGAAATGGTTGGTATCTCT
GAACTGGCTGCTCTGATCGCTGGTGAAAAACGTGA
AGAAAACGGTGCTATCAAACGTATGATCGAACAGG
ACAAAGGTTCTTCTGACTTCTTCACCTACGGTGCT
AACCTGACCTTCGTTAACCTGGACGAAATCGACAT
GTACGAACTGGAAATCAACGGTGGTAAACCGGACT
TCGTTAACTACACCATCCACGGTGTTGGTGACAAA
GGTGTTGTTCTGGTTTTCCCGAAACAGAACTTCGC
TCGTATCGTTTCTGTTGTTATGCCGGAAGAAGACC
TGGCTAAACTGAAAGAAGAAGTTACCAACATGATC
ATCTAA
SEQ ID NO: 17
MVDKRESYTKEDLLASGRGELFGAKGPQLPAPNML
MMDRVVKMTETGGNFDKGYVEAELDINPDLWFFGC
HFIGDPVMPGCLGLDAMWQLVGFYLGWLGGEGKGR
ALGVGEVKFTGQVLPTAKKVTYRIHFKRIVNRRLI
MGLADGEVLVDGRLIYTASDLKVGLFQDTSAF

What is claimed is:

1. A biomanufacturing system for producing an organic product comprising:
at least one bioreactor culture vessel;
wherein the at least one bioreactor culture vessel contains an organic substrate culture solution,
wherein the organic substrate culture solution contains a first recombinant microorganism having an improved organic substrate producing ability,
wherein the first recombinant microorganism expresses at least one organic substrate forming recombinant enzyme by expressing at least one non-native organic substrate forming enzyme nucleotide sequence,
wherein the first recombinant microorganism is capable of utilizing a carbon source to produce the organic substrate,
wherein the first recombinant microorganism produces at least one organic substrate culture impurity, including oxygen, at least a byproduct in gas, a liquid, and a solid phase; and
wherein the at least one bioreactor culture vessel contains an organic product culture solution,
wherein the organic product culture solution contains a second recombinant microorganism having an improved organic product producing ability,
wherein the second recombinant organism expresses at least one organic product forming enzyme by expressing at least one non-native organic product forming enzyme nucleotide sequence,
wherein the second recombinant organism is capable of utilizing the organic substrate to produce the at least one organic product.

2. The system of claim 1, wherein the at least one bioreactor culture vessel includes a first bioreactor culture vessel including a carbon source inlet, and a power source; and a second bioreactor culture vessel including a fluid flow path connected to and between the first bioreactor culture vessel and the second bioreactor culture vessel, and an organic product outlet;
wherein the first bioreactor culture vessel includes the organic substrate culture solution, and the second bioreactor culture vessel includes the organic product culture solution; or
wherein the first bioreactor culture vessel or the second bioreactor culture vessel further comprises a biomass collection port.

3. The system of claim 2, wherein the carbon source includes carbon dioxide, carbon monoxide, glycerol, glucose, fructose, sucrose, a monosaccharide, a disaccharide, a polysaccharide, glycogen, acetic acid, a fatty acid, or a combination thereof; or
wherein the power source includes sunlight, a solar power source, an electrical power source, or a combination thereof; or
wherein the volatile gas includes oxygen, methane, or a combination thereof; or
wherein the at least one organic substrate includes alpha-ketoglutarate, sucrose, glucose, fructose, xylose, arabinose, galactose, glycerol, a monosaccharide, a disaccharide, a polysaccharide, glycogen, a fatty acid, or a combination thereof; or
wherein the at least one organic product includes an alcohol, methanol, ethanol, propanol, butanol, ethane diol, an organic acid, propionic acid, acetic acid, an aldehyde, formaldehyde, a long chain fatty acid, an n-alkane, a hydrocarbon, ethane, propene, butene, ethane, propane, butane, C₂-C₂₀ alkane, C₂-C₂₀ alkene, or a combination thereof; or
wherein the second bioreactor culture vessel further includes a carbon source inlet, a volatile gas outlet, a power source, or a combination thereof.

4. The system of claim 1, wherein the organic substrate culture solution and the organic product culture solution are combined in one bioreactor culture vessel; or
the organic substrate culture solution and the organic product culture are separated by a filter wherein the filter includes a pore size of from about 0.2 μm to about 10 μm or more; or wherein the system further comprises a carbonation unit, an amine stripper, an amine scrubber, a catalytic converter, a condenser, a compressor, a caustic tower, a dryer, or combinations thereof.

5. The system of claim 1, wherein the organic substrate includes alpha-ketoglutarate (AKG), wherein an amount of the at least one AKG forming enzyme produced by the first recombinant microorganism is greater than that produced relative to a control microorganism lacking a non-native AKG forming enzyme expressing nucleotide sequence; or
wherein the at least one organic product forming enzyme includes ethylene forming enzyme (EFE), and an amount of EFE produced by the second recombinant microorganism is greater than that produced relative to a control microorganism lacking a non-native EFE expressing nucleotide sequence.

6. The system of claim 5, wherein the first recombinant microorganism expresses at least one alpha-ketoglutarate permease protein (AKGP) by expressing at least one non-native AKGP forming nucleotide sequence.

7. The system of claim 5, wherein the at least one AKG forming enzyme includes an isocitrate dehydrogenase (ICD) protein, a glutamate dehydrogenase (GDH) protein, or a combination thereof.

8. The system of claim 7, wherein the first recombinant microorganism expresses an ICD protein having an amino acid sequence at least 95% identical to SEQ ID NO: 1 by expressing a non-native ICD protein nucleotide sequence having a nucleotide sequence at least 95% identical to SEQ ID NO: 2; or
the first recombinant microorganism expresses a GDH protein having an amino acid sequence at least 95% identical to SEQ ID NO: 5 by expressing a non-native GDH protein nucleotide sequence having a nucleotide sequence at least 95% identical to SEQ ID NO: 6, or
a combination thereof; or wherein the second recombinant microorganism expresses an EFE protein having an amino acid sequence at least 95% identical to SEQ ID NO: 7 by expressing a non-native EFE protein nucleotide sequence having a nucleotide sequence at least 95% identical to SEQ ID NO: 8.

9. The system of claim 1, wherein the first recombinant microorganism includes a microorganism selected from the group consisting of a photosynthetic bacterium, a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, and Chlamydomonas reinhardtii; or
wherein the second recombinant microorganism includes a microorganism selected from the group consisting of Escherichia, Escherichia coli, Geobacteria, Arthrobacter paraffineus, Pseudomonas fluorescens, Pseudomonas Putida, a Pseudomonas, Pseudomonas syringae, Pseudomonas savastanoi, Serratia marcescens, Bacillus metatherium, Candida paludigena, Pichia inositovora, Torulopsis glabrata, Candida lipolytica, Yarrowia lipolytica, Saccharomyces cereviciae, Aspergillus sp., Bacillus subtilis, and Lactobacillus sp.

10. The system of claim 1, wherein the first recombinant microorganism includes a delta-glgc mutant microorganism lacking expression of a glucose-1-phosphate adenylyltransferase protein; or wherein the first recombinant microorganism expresses a sucrose synthase protein having an amino acid sequence at least 95% identical to SEQ ID NO: 9 by expressing a non-native sucrose synthase protein nucleotide sequence having a nucleotide sequence at least 95% identical to SEQ ID NO: 10; or wherein the first recombinant microorganism expresses a sucrose phosphate synthase protein having an amino acid sequence at least 95% identical to SEQ ID NO: 11 by expressing a non-native sucrose phosphate synthase nucleotide sequence having a nucleotide sequence at least 95% identical to SEQ ID NO: 12.

11. The system of claim 5, wherein an amount of at least one AKG forming enzyme produced by the first recombinant microorganism is from about 5% to about 200% or more greater than that produced relative to the control microorganism lacking the non-native AKG forming enzyme expressing nucleotide sequence; or
wherein an amount of EFE protein produced by the second recombinant microorganism is from about 5% to about 200% or more greater than that produced relative to the control microorganism lacking the non-native EFE expressing nucleotide sequence; or
wherein an amount of EFE protein produced by the second recombinant microorganism is from about 20 grams to about 100 grams per liter or more of organic product culture solution.

12. The system of claim 5, wherein the second recombinant microorganism includes E. coli, and an amount of EFE protein produced by the second recombinant microorganism is from about 30% to about 80% or more of a total cellular amount of protein of the second recombinant microorganism; or
a production rate of the at least one organic product produced by the second recombinant microorganism is from about 100 million pounds/year to about 1 billion pounds/year or more; or wherein a cell population concentration of the second recombinant microorganism ranges from about 10⁷ to about 10¹³ cells per milliliter, or a dry cell weight per liter of organic product culture solution of from about 100 grams to about 300 grams dry cell weight per liter.

13. The system of claim 5, wherein the non-native AKG forming enzyme expressing nucleotide sequence or the non-native EFE expressing nucleotide sequence is inserted into a microbial expression vector, wherein the microbial expression vector includes a bacterial vector plasmid, a nucleotide guide of a homologous recombination system, an antibiotic-resistant system, an aid system for protein purification and detection, a CRISPR CAS system, a phage display system, or a combination thereof.

14. The system of claim 5, wherein the EFE expressing nucleotide sequence has a copy number in the microbial expression vector of from about 2 to about 500; or wherein the microbial expression vector includes at least one microbial expression promoter.

15. The system of claim 14, wherein the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof.

16. A method of producing an organic product comprising:
providing a biomanufacturing system as in claim 2;
culturing the first recombinant microorganism in the first bioreactor culture vessel under conditions sufficient to produce an amount of the at least one organic substrate in the first bioreactor culture vessel; and
culturing the second recombinant microorganism in the second bioreactor culture vessel under conditions sufficient to produce an amount of the at least one organic product in the second bioreactor culture vessel.

17. The method of claim 16, further comprising:
removing an amount of the at least one volatile gas from the organic substrate culture solution through the volatile gas outlet;
removing an amount of the at least one organic product from the organic product culture solution through the organic product outlet;
provided the carbon source includes carbon dioxide, feeding an amount of the carbon dioxide from a carbon dioxide source into the organic substrate culture solution through the carbon source inlet;
maintaining a pH level of the organic substrate culture solution and the organic product culture solution from about 5.0 to about 8.5;
maintaining the organic substrate culture solution and the organic product culture solution at a temperature of from about 25 degrees Celsius to about 70 degrees Celsius;
provided the first bioreactor culture vessel or the second bioreactor culture vessel includes a biomass collection port, collecting an amount of biomass produced by the first recombinant microorganism or the second recombinant microorganism through the biomass collection port; or
maintaining an amount of volatile gas in the second bioreactor culture vessel of from about 10% by volume to about 1% by volume or less, based on a total internal volume of the second bioreactor culture vessel.

18. The method of claim 16, wherein the non-native organic substrate forming recombinant enzyme expressing nucleotide sequence or the non-native organic product expressing nucleotide sequence is inserted into a microbial expression vector, wherein the at least one microbial expression vector includes at least one microbial expression promoter, further comprising:
controlling the amount of the at least one organic substrate or the amount of the at least one organic product produced by adding at least one promoter inducer to the organic substrate culture solution or the organic product culture solution.

19. The method of claim 18, wherein the at least one microbial expression promoter includes a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof; and the at least one promoter inducer includes lactose, xylose, IPTG, cold shock, heat shock, or a combination thereof.

20. The method of claim 16, further comprising:
provided the at least one organic substrate culture impurity includes at least one volatile gas, removing the at least one volatile gas through the volatile gas outlet; or
recovering an amount of at least one organic product produced at a rate of from about 100 million pounds/year to about 1 billion pounds/year or more; or
wherein the amount of the at least one organic product produced contains an amount of volatile gas of about 1 mole percent or less.

21. A method of producing an organic product comprising:
providing a bioremediation system as in claim 1, wherein the organic substrate culture solution and the organic product culture solution are combined in one bioreactor culture vessel, wherein the non-native organic substrate forming recombinant enzyme expressing nucleotide sequence is inserted into a first microbial expression vector, wherein the non-native organic product forming enzyme expressing nucleotide sequence is inserted into a second microbial expression vector, wherein the first and second microbial expression vector each includes at least one microbial expression promoter,
providing a carbon source connected to the carbon source inlet;
culturing the first recombinant microorganism in the first bioreactor culture vessel under conditions sufficient to produce an amount of the at least one organic substrate in the first bioreactor culture vessel;
producing the amount of the at least one organic substrate by adding at least one promoter inducer to the organic substrate culture solution at a first time point;
culturing the second recombinant microorganism in the second bioreactor culture vessel under conditions sufficient to produce an amount of the at least one organic product in the second bioreactor culture vessel; and
producing the amount of the at least one organic product by adding at least one promoter inducer to the organic product culture solution at a second time point.

22. The method of claim 21, further comprising lowering an amount of oxygen in the second bioreactor culture vessel to from about 10% by volume to about 1% by volume or less, based on a total internal volume of the second bioreactor culture vessel, before the second time point.


SEQUENCE LISTING (17 sequences)

SEQ ID NO: 1 - protein, 418 aa, Pseudomonas fluorescens
MGYKKIQVPAVGDKITVNADHSLNVPDNPIIPFIEGDGIGVDVSPVMIKVVDAAVEKAYGGKRKISWMEVYAGEKATQVYDQDTWLPQETLDAVKDYVVSIKGPLTTPVGGGIRSLNVALRQQLDLYVCLRPVVWFEGVPSPVKKPGDVDMVIFRENSEDIYAGIEWKAGSPEATKVIKFLKEEMGVTKIRFDQDCGIGIKPVSKEGTKRLVRKALQYVVDNDRKSLTIVHKGNIMKFTEGAFKDWGYEVAKEEFGAELLDGGPWMKFKNPKTGREVVVKDAIADAMLQQILLRPAEYDVIATLNLNGDYLSDALAAEVGGIGIAPGANLSDTVAMFEATHGTAPKYAGKDQVNPGSVILSAEMMLRHLGWTEAADLIIKGTNGAIKAKTVTYDFERLMEGATLVSSSGFGEALIKHM

SEQ ID NO: 2 - DNA, 1257 bp, Pseudomonas fluorescens
atgggttacaagaagattcaggttccagccgtcggcgacaaaatcaccgtcaacgcagaccattctctcaatgtccctgataacccgatcattcccttcatcgaaggtgacggcattggcgtcgacgtcagccctgtgatgatcaaagtggttgatgctgccgtagagaaagcctacgggggtaagcgcaagatttcctggatggaggtttatgctggcgaaaaagcaactcaggtctatgaccaggacacctggctgccccaggaaaccctggacgcggtcaaggattacgtggtctccatcaaaggcccgctgaccactccggtcggtggcggcatccgttccctcaacgtcgccctgcgccaacagctcgatctctatgtctgccttcgccctgtggtgtggttcgaaggtgtgccgagcccggtgaaaaagcctggcgacgtcgacatggtgatcttccgcgagaactccgaagacatttatgccggtatcgaatggaaagccggctcccctgaggccaccaaggtcatcaaattcctgaaagaagaaatgggcgtcaccaagatccgtttcgaccaggattgcggcatcggcatcaagccggtttccaaagaaggcaccaagcgtctggtgcgcaaggcgctgcaatacgtggtggacaacgaccgcaagtcgctgaccatcgtgcacaagggcaacatcatgaaattcaccgaaggtgccttcaaggactggggctacgaggtggcgaaggaagaattcggcgccgagctgctcgatggcggcccatggatgaaattcaagaacccgaaaaccggccgcgaagtcgtcgtcaaggacgccatcgccgacgccatgctccagcagatcctgctgcgtccggccgaatacgatgtgatcgccaccctcaacctcaacggtgactacctgtccgacgccctggcggcggaagtgggcggtatcggtatcgcgccgggtgccaacctgtccgacaccgtagccatgttcgaggcgacccacggtactgcgccgaaatatgccggcaaggaccaggtcaacccgggttcggtgattttgtcggcggaaatgatgctgcgccacctgggctggaccgaggcggccgacctgatcatcaagggcaccaatggcgccatcaaggccaagaccgtgacctacgacttcgaacgtctgatggaaggcgccacactggtgtcttcttcgggcttcggtgaagcgctgatcaagcacatgtaa

SEQ ID NO: 3 - protein, 475 aa, Synechococcus elongatus
MYEKIQPPSEGSKIRFEAGKPIVPDNPIIPFIRGDGTGVDIWPATERVLDAAVAKAYGGQRKITWFKVYAGDEACDLYGTYQYLPEDTLTAIREYGVAIKGPLTTPIGGGIRSLNVALRQIFDLYACVRPCRYYTGTPSPHRTPEQLDVVVYRENTEDIYLGIEWKQGDPTGDRLIKLLNEDFIPNSPSLGKKQIRLDSGIGIKPISKTGSQRLIRRAIEHALRLEGRKRHVTLVHKGNIMKFTEGAFRDWGYELATTEFRTDCVTERESWILANQESKPDLSLEDNARLIEPGYDAMTPEKQAAVVAEVKAVLDSIGATHGNGQWKSKVLVDDRIADSIFQQIQTRPGEYSVLATMNLNGDYISDAAAAVVGGLGMAPGANIGDEAAIFEATHGTAPKHAGLDRINPGSVILSGVMMLEYLGWQEAADLITKGISQAIANREVTYDLARLMEPAVDQPLKCSEFAEAIVKHFDD

SEQ ID NO: 4 - DNA, 1450 bp, Synechococcus elongatus
agggcaagcttatgtacgagaagattcaaccccctagcgaaggcagcaaaattcgctttgaagccggcaagccgatcgttcccgacaacccgatcattcccttcattcgtggtgacggcactggcgttgatatctggcccgcaactgagcgcgttctcgatgccgctgtcgctaaagcctatggcggtcagcgcaaaatcacttggttcaaagtctacgcgggtgatgaagcctgcgacctctacggcacctaccaatatctgcctgaagatacgctgacagcgatccgcgagtacggcgtggcaatcaaaggcccgctgacgacgccgatcggtggtggcattcgatcgctgaacgtggcgctacggcaaatcttcgatctctatgcctgcgtccgcccctgtcgctactacaccggcacaccctcgccccaccgcacgcccgaacaactcgatgtggtggtctaccgcgaaaacaccgaggatatctacctcggcatcgaatggaagcaaggtgatcccaccggcgatcgcctgatcaagctgctgaacgaggacttcattcccaacagccccagcttgggtaaaaagcaaatccgtttggattccggcattggtattaagccgatcagtaaaacgggtagccagcgtctgattcgtcgtgcgatcgagcatgccctacgcctcgaaggccgcaagcgacatgtcacccttgtccacaagggcaacatcatgaagttcacggaaggtgctttccgggactggggctatgaactggccacgactgagttccgaaccgactgtgtgactgaacgggagagctggattcttgccaaccaagaaagcaagccggatctcagcttggaagacaatgcgcggctcatcgaacctggctacgacgcgatgacgcccgaaaagcaggcagcagtggtggctgaagtgaaagctgtgctcgacagcatcggcgccacccacggcaacggtcagtggaagtctaaggtgctggttgacgatcgcattgctgacagcatcttccagcagattcaaacccgcccgggtgaatactcggtgctggcgacgatgaacctcaatggcgactacatctctgatgcagcggcggcggtggtcggtggcctgggcatggcccccggtgccaacattggcgacgaagcggcgatctttgaagcgacccacggcacagcgcccaagcacgctggcctcgatcgcattaaccccggctcggtcatcctctccggcgtgatgatgctggagtacctaggctggcaagaggctgctgacttgatcaccaagggcatcagccaagcgatcgctaaccgtgaggtcacctacgatctggctcggttgatggaaccggcggttgatcaaccactcaagtgctcggaatttgccgaagccatcgtcaagcatttcgacgattagggatccagcgc

SEQ ID NO: 5 - protein, 1628 aa, Pseudomonas fluorescens
MAFFTAASKADFQHQLQAALAQHISEQALPQVALFAEQFFGIISLDELTQRRLSDLAGCTLSAWRLLERFDHAQPQVRVYNPDYERHGWQSTHTAVEVLHHDLPFLVDSVRTELNRRGYSIHTLQTTVLSVRRGSKGELLEILPKGTTGEGVLHESLMYLEIDRCANAAELNVLSKELEQVLGEVRVAVSDFEPMKAKVQEILTKLDNSAFAVDADEKNEIKSFLEWLVGNHFTFLGYEEFTVVDQADGGHIEYDQNSFLGLTKMLRTGLTNEDRHIEDYAVKYLREPTLLSFAKAAHPSRVHRPAYPDYVSIREIDADGKVIKEHRFMGLYTSSVYGESVRVIPFIRRKVEEIERRSGFQAKAHLGKELAQVLEVLPRDDLFQTPVDELFSTVMSIVQIQERNKIRVFLRKDPYGRFCYCLAYVPRDIYSTEVRQKIQQVLMERLKASDCEFWTFFSESVLARVQLILRVDPKNRIDIDPLQLENEVIQACRSWQDDYAALTVETFGEANGTNVLADFPKGFPAGYRERFAAHSAVVDMQHLLNLSEKKPLAMSFYQPLASGPRELHCKLYHADTPLALSDVLPILENLGLRVLGEFPYRLRHTNGREFWIHDFAFTAAEGLDLDIQQLNDTLQDAFVHIVRGDAENDAFNRLVLTAGLPWRDVALLRAYARYLKQIRLGFDLGYIASTLNNHTDIARELTRLFKTRFYLARKLGSEDLDDKQQRLEQAILTALDDVQVLNEDRILRRYLDLIKATLRTNFYQTDANGQNKSYFSFKFNPHLIPELPKPVPKFEIFVYSPRVEGVHLRFGNVARGGLRWSDREEDFRTEVLGLVKAQQVKNSVIVPVGAKGGFLPRRLPLGGSRDEIAAEGIACYRIFISGLLDITDNLKDGKLVPPANVVRHDDDDPYLVVAADKGTATFSDIANGIAIDYGFWLGDAFASGGSAGYDHKKMGITAKGAWVGVQRHFRERGINVQEDSITVVGVGDMAGDVFGNGLLMSDKLQLVAAFNHLHIFIDPNPNPATSFAERQRMFDLPRSAWSDYDTSIMSEGGGIFSRSAKSIAISPQMKERFDIQADKLTPTELLNALLKAPVDLLWNGGIGTYVKASTESHADVGDKANDALRVNGNELRCKVVGEGGNLGMTQLGRVEFGLNGGGSNTDFIDNAGGVDCSDHEVNIKILLNEVVQAGDMTDKQRNQLLASMTDEVGGLVLGNNYKQTQALSLAARRAYARIAEYKRLMSDLEGRGKLDRAIEFLPTEEQLAERVAEGHGLTRPELSVLISYSKIDLKEQLLGSLVPDDDYLTRDMETAFPPTLVSKFSEAMRRHRLKREIVSTQIANDLVNHMGITFVQRLKESTGMTPANVAGAYVIVRDIFHLPHWFRQIEALDYQVSADVQLELMDELMRLGRRATRWFLRARRNEQNAARDVAHFGPHLKELGLKLDELLSGEIRENWQERYQAYVAAGVPELLARMVAGTTHLYTLLPIIEAADVTGQDPAEVAKAYFAVGSALDITWYISQISALPVENNWQALAREAFRDDVDWQQRAITIAVLQAGGGDSDVETRLALWMKQNDAMIERWRAMLVEIRAASGTDYAMYAVANRELNDVALSGQAVVPAAATAELELA

SEQ ID NO: 6 - DNA, 4887 bp, Pseudomonas fluorescens
atggcgttcttcaccgcagccagcaaagccgacttccagcaccaactgcaagcggcactggcgcagcacatcagtgaacaggcactgccacaagtggcgctgttcgctgaacaattcttcggcatcatttccctggacgagctgacccaacgtcgcctctccgacctcgctggctgtactctctctgcgtggcgcctgcttgagcgcttcgatcacgcgcaaccgcaagtgcgcgtctacaaccccgattacgaacgtcacggctggcagtcgacccacaccgcggtcgaagtgctgcaccacgacttgccgttcctggtggactcggtgcgtaccgagctgaaccgtcgcggctacagcatccacaccctgcagaccaccgtgctgagcgtgcgtcgtggcagcaagggcgaattgctggaaatcctgccaaaaggcaccaccggcgaaggcgttctgcacgagtcgctgatgtacctggaaatcgaccgctgcgccaatgcggccgaattgaatgtgctgtccaaggaactggagcaggtcctgggtgaagtccgcgtcgcggtctccgatttcgagccgatgaaggccaaggtgcaggaaatcctcaccaagctcgataacagcgcattcgccgtcgatgccgacgaaaagaatgaaatcaagagcttcctggaatggctggtgggcaaccacttcaccttcctcggctacgaagagttcaccgttgtcgatcaggccgatggcggccacatcgaatacgaccagaactccttcctcggcctgaccaagatgctgcgcaccggtctgaccaacgaagaccgccacatcgaagactatgccgtgaagtacctgcgcgaaccgacactgctgtcgttcgccaaggcggcgcatccgagccgcgtgcaccgtccggcctacccggactacgtgtcgatccgcgaaatcgatgccgacggcaaagtgatcaaggaacaccgcttcatgggcctgtacacctcgtcggtgtatggcgaaagcgtgcgtgtcatcccgttcatccgccgcaaggtcgaggaaatcgagcgtcgctccggcttccaggccaaggctcacctgggcaaggaactggctcaggttctggaagtgctgccgcgtgacgatctgttccagaccccggtcgacgaactgttcagcaccgtgatgtcgatcgtgcagatccaggaacgcaacaagatccgcgtgttcctgcgtaaagacccgtacggtcgtttctgctactgcctggcctacgtgccgcgtgacatctactccaccgaagttcgccagaagatccagcaagtgctgatggagcgcctgaaagcctccgactgcgaattctggacgttcttctccgagtccgtgctggcccgcgtgcaactgatcttgcgcgtcgacccgaaaaaccgcatcgacatcgacccgctgcaactggaaaacgaagtgatccaggcctgccgcagctggcaggacgactacgctgccctgaccgttgaaaccttcggcgaagccaacggcaccaacgtgttggccgacttcccgaaaggcttcccggccggctaccgcgagcgtttcgcagcgcattcggccgtggtcgacatgcagcacttgctcaatctgagcgagaaaaagccgctggccatgagcttttaccagccgctggcctccggcccacgcgagctgcactgcaagctgtatcacgccgataccccgctggccctgtccgacgtgctgccgatcctggaaaacctcggcctgcgcgtgctgggtgagttcccgtaccgcctgcgtcataccaacggccgcgagttctggatccacgacttcgcgttcaccgctgccgaaggcctggacctggacatccagcaactcaacgacaccctgcaggacgcgttcgtccacatcgtccgtggcgatgccgaaaacgatgcgttcaaccgtctggtgctgaccgccggcctgccatggcgcgacgtggcgctgctgcgtgcctacgcccgctacctgaagcagatccgcctgggcttcgacctcggctacatcgccagcaccctgaacaaccacaccgacatcgctcgcgaactgacccggttgttcaagacccgtttctacctggcccgcaagctgggcagcgaggatctggacgacaagcaacagcgtctggaacaggccatcctgaccgcgctggacgacgttcaagtcctcaacgaagaccgcatcctgcgtcgttacctggacctgatcaaagcaaccctgcgcaccaacttctaccagaccgacgccaacggccagaacaagtcgtacttcagcttcaagttcaacccgcacttgattcctgaactgccgaaaccggtgccgaagttcgaaatcttcgtttactcgccacgcgtcgaaggcgtgcacctgcgcttcggcaacgttgctcgtggtggtctgcgctggtcggaccgtgaagaagacttccgtaccgaagtcctcggcctggtaaaagcccagcaagtgaagaactcggtcatcgtgccggtgggggcgaagggcggcttcctgccgcgtcgcctgccactgggcggcagccgtgacgagatcgcggccgagggcatcgcctgctaccgcatcttcatttcgggcctgttggacatcaccgacaacctgaaagacggcaaactggtaccgccggccaacgtcgtgcggcatgacgacgatgacccgtacctggtggtcgcggcggacaagggcactgcaaccttctccgacatcgccaacggcatcgccatcgactacggcttctggctgggtgacgcgttcgcgtccggtggttcggccggttacgaccacaagaaaatgggcatcaccgccaagggcgcgtgggtcggcgtacagcgccacttccgcgagcgcggcatcaatgtccaggaagacagcatcacggtggtcggcgtgggcgacatggccggtgacgtgttcggtaacggcctgttgatgtctgacaagctgcaactggttgctgcgttcaaccacctgcacatcttcatcgacccgaacccgaacccggccaccagcttcgccgagcgtcagcgcatgttcgatctgccgcgctcggcctggtccgactacgacaccagcatcatgtccgaaggcggcggcatcttctcgcgcagcgcgaagagcatcgccatctcgccacagatgaaagagcgcttcgacatccaggccgacaagctgaccccgaccgaactgctgaacgccttgctcaaggcgccggtggatctgctgtggaacggcggtatcggtacctacgtcaaagccagcaccgaaagtcacgccgatgtcggcgacaaggccaacgatgcgctgcgcgtgaacggcaacgaactgcgctgcaaagtggtgggcgagggcggtaacctcggcatgacccaattgggtcgtgtggagttcggtctcaatggcggcggttccaacaccgacttcatcgacaacgccggtggcgtggactgctccgaccacgaagtgaacatcaagatcctgctgaacgaagtggttcaggccggcgacatgaccgacaagcaacgcaaccagttgctggcgagcatgaccgacgaagtcggtggtctggtgctgggcaacaactacaagcagactcaggccctgtccctggcggcccgccgtgcttatgcgcggatcgccgagtacaagcgtctgatgagcgacctggagggccgtggcaagctggatcgcgccatcgagttcctgccgaccgaagagcaactggccgaacgcgttgccgaaggccatggcctgacccgtcctgagctgtcggtgctgatctcgtacagcaagatcgacctcaaggagcagctgctgggctccctggtgccggacgacgactacctgacccgcgacatggaaacggcgttcccgccgaccctggtcagcaagttctccgaagctatgcgtcgtcaccgcctcaagcgcgagatcgtcagcacccagatcgccaacgatctggtcaaccacatgggcatcaccttcgttcagcgactcaaagagtccacgggcatgaccccggcgaatgttgccggtgcgtatgtgattgttcgggatatcttccacctcccgcactggttccgtcagatcgaagcgctggactaccaggtttccgctgacgtgcagctggagctgatggacgagctgatgcgtctgggccgtcgcgctacgcgctggttcctgcgtgcccgtcgcaacgagcagaacgctgcccgtgacgtcgcgcatttcggtccgcacctcaaagagctgggcctgaagctggacgagctgctgagcggcgaaatccgcgaaaactggcaagagcgttatcaggcgtacgtcgccgccggtgttccggaactgctggcgcgtatggtggcggggacgacccacctctacacgctgctgccgatcatcgaagcggccgacgtgaccggccaggatccagccgaagtggccaaggcgtacttcgccgtgggcagcgcgctggacatcacctggtacatctcgcagatcagcgccttgccggttgaaaacaactggcaggccctggcccgtgaagcgttccgcgacgacgtcgactggcagcaacgcgcgattaccatcgccgttctgcaagcgggtggcggtgattcggacgtggaaacccgtctggcactgtggatgaagcagaacgacgccatgatcgaacgctggcgcgccatgctggtggaaatccgtgccgccagcggcaccgactacgccatgtacgcggtggccaaccgcgagctgaacgacgtggcgctgagcggtcaggcagttgtgcctgctgcggcgactgcggagcttgagcttgcttga

SEQ ID NO: 7 - protein, 387 aa, Escherichia coli
MIHAPSRWGVFPSLGLCSPDVVWNEHPSLYMDKEETSMTNLQTFELPTEVTGCAADISLGRALIQAWQKDGIFQIKTDSEQDRKTQEAMAASKQFCKEPLTFKSSCVSDLTYSGYVASGEEVTAGKPDFPEIFTVCKDLSVGDQRVKAGWPCHGPVPWPNNTYQKSMKTFMEELGLAGERLLKLTALGFELPINTFTDLTRDGWHHMRVLRFPPQTSTLSRGIGAHTDYGLLVIAAQDDVGGLYIRPPVEGEKRNRNWLPGESSAGMFEHDEPWTFVTPTPGVWTVFPGDILQFMTGGQLLSTPHKVKLNTRERFACAYFHEPNFEASAYPLFEPSANERIHYGEHFTNMFMRCYPDRITTQRINKENRLAHLEDLKKYSDTRATGS

SEQ ID NO: 8 - DNA, 1161 bp, Artificial Sequence
  Description of Artificial Sequence Synthetic polynucleotide
atgatacacgctccaagtagatggggagtatttccctcactagggttatgcagcccggacgttgtgtggaatgagcatccgagcctgtacatggacaaagaggaaaccagcatgaccaacctgcagacctttgaactgccgaccgaagtgaccggttgcgcggcggacatcagcctgggtcgtgcgctgattcaggcgtggcaaaaggatggtatcttccagattaaaaccgacagcgagcaggatcgtaagacccaagaagcgatggcggcgagcaagcaattttgcaaagagccgctgaccttcaaaagcagctgcgttagcgacctgacctacagcggttatgtggcgagcggcgaggaagttaccgcgggcaagccggatttcccggaaatttttaccgtgtgcaaggacctgagcgtgggcgatcagcgtgttaaagcgggttggccgtgccatggtccggttccgtggccgaacaacacctatcaaaagagcatgaaaacctttatggaggaactgggtctggcgggcgagcgtctgctgaaactgaccgcgctgggttttgaactgccgatcaacaccttcaccgacctgacccgtgatggctggcaccacatgcgtgtgctgcgtttcccgccgcagaccagcaccctgagccgtggtattggtgcgcacaccgactacggtctgctggtgattgcggcgcaagacgatgttggtggcctgtatatccgtccgccggtggagggcgaaaagcgtaaccgtaactggctgccgggcgagagcagcgcgggcatgtttgagcacgacgaaccgtggaccttcgttaccccgaccccgggtgtgtggaccgtttttccgggcgatattctgcagttcatgaccggtggccaactgctgagcaccccgcacaaggttaaactgaacacccgtgaacgtttcgcgtgcgcgtactttcacgagccgaacttcgaagcgagcgcgtatccgctgttcgagccgagcgcgaacgaacgtatccactacggcgagcacttcaccaacatgtttatgcgttgctatccggatcgtatcaccacccaacgtattaacaaagaaaaccgtctggcgcacctggaagacctgaagaaatacagcgacacccgtgcgaccggcagc

SEQ ID NO: 9 - protein, 808 aa, Anabaena sp.
MYKLVQTIVNSDEKNVLGDFILELGKDHKRYFLRNEILQAFADYCHQFPKPAYFYHSSSLGTFIQYTHEIILDGENTWFVVRPKIASQEVWLLSADLTKFELMTPKALLDVSDRLVKRYQPHILEIDLHPFYSAAPRIDDSRNIGQGLTVLNHYFCNQALTDPEYWIDALFQSLKRLEYNGIKLLISNHIHSGLQLTKQIKLALEFVSHLSPQTPYIKFKFHLQELGLEPGWGNNAARVRETLELLERLMDNPEPAILETFVSRICAVFRVVLISIHGWVAQEDVLGRDETLGQVIYVLEQARSLENKMRAEIKLAGLDTLGIKPHIIILTRLIPNCEGTFCNLPLEKVDGTENAWILRVPFAESRPEITNNWISKFEIWPYLEKFALDAEAELLKQFQGKPNLIIGNYSDGNLVAFILSRKMKVTQCNIAHSLEKPKYLFSNLYWQDLEAQYHFSAQFTADLISMNAADFIITSSYQEIVGTPDTMGQYESYKCFTMPNLYHVIDGIDLFSPKFNVVLPGVSENIFFPYNQTTNRESHRRQHIQDLIFHQEHPEILGKLDHPHKKPIFSVSPITSIKNLTGLVECFGKSEELQKHSNLILLTSKLHPDLGTNSEEIQEIAKIHAIIDQYHLHHKIRWLGMRLPLRDIAETYRVIADFQGIYIHFALYESFSRSILEAMISGLPTFTTQFGGSLEIIENHDQGFNLNPTDLAGTAKTIINFLEKCENYPEHWLENSQWMIERIRHKYNWNSHTNQLLLLTKMFSFWNFIYPEDNEARDRYMESLFHLLYKPIADHILSEHLSKIRNHN

SEQ ID NO: 10 - DNA, 2427 bp, Anabaena sp.
atgtataaattagtgcaaactattgttaacagtgatgaaaaaaatgttttaggtgactttatcttagaattaggcaaggatcataaacgttactttttaagaaatgagattttacaagcttttgcagattattgtcaccaattcccaaaacccgcttatttttatcactcttcctctttagggacattcattcaatacacccatgaaataattttagatggtgaaaatacttggtttgtagttagaccaaagattgcgagtcaagaagtatggttattaagcgcggacttgactaagtttgagttaatgacaccgaaagcattattagatgtgagcgatcgcttagtaaagcgttatcaaccgcacattttagaaattgatctccatcccttttattcagcagcaccaagaattgatgattccagaaatattggccaaggtttaaccgttcttaatcattatttttgtaatcaagcattgacagatcctgaatattggattgacgcattatttcaatcattaaaaagattagaatataacggcatcaaattattaattagtaatcatattcattcaggtttgcaactaacaaagcaaatcaaactagcgttagaatttgtgagtcatttatccccccagacaccatatataaaatttaaatttcatcttcaagaactcggtttagaaccaggttggggtaataatgcagccagagtcagagaaaccttagaactgctggaaagactcatggataatcccgaacctgcaattttagaaacctttgtttctcgcatttgtgcagttttccgcgtcgtccttatttccatccatggttgggttgcacaagaagatgttttaggcagagatgaaacattaggacaagttatttatgttttagaacaagcccgcagtttagaaaataaaatgcgggcagaaattaaacttgcaggtttagatacattaggaattaaaccccatatcattatattaactcgactgattcccaattgtgaaggcacattttgtaacttaccattagaaaaagttgatggtacagaaaatgcttggattttgcgcgttccttttgcagaatctcgaccggaaattaccaacaactggatttctaaatttgaaatttggccttatttagaaaaatttgctcttgatgccgaagcagaacttttaaaacaattccaaggaaagcccaatctaattattggtaactacagtgacgggaacttagttgcttttattctctcccgaaaaatgaaagttacccaatgtaatattgcccattccctcgaaaaacctaaatatctatttagtaacttatattggcaagatttagaagcacaatatcacttttctgcccaatttaccgctgatttaatcagtatgaatgccgcagattttattatcacatcatcctatcaagaaattgtaggtacaccagatacaatgggacaatatgaatcttataaatgtttcaccatgcccaacttatatcatgtaattgatggcattgatttatttagccctaaattcaatgtggtattaccaggagtcagtgaaaatatattttttccctacaaccaaacaacaaatagagaatcccaccgtcgtcaacatatccaagacctaattttccatcaagaacacccagaaattctcggtaaattagatcatcctcataaaaaaccgatcttttccgttagtcccattacctcaattaaaaacctcacaggtttagttgaatgtttcggtaaaagtgaagaattacaaaaacatagtaacctaattttattaaccagtaaacttcatccagacttaggaacaaactccgaagaaattcaagaaatagcaaaaattcatgcgattattgatcaatatcatcttcaccataaaatccgctggttgggaatgcgtcttcctctccgcgatattgctgaaacctatcgtgtaattgccgattttcaagggatttatattcactttgccctttatgaatcctttagcagaagtattttagaagcaatgatttctggattaccaacttttacaactcaatttggtggttcattagaaattattgaaaaccatgatcaaggatttaacctcaaccccacagacttagcaggaacagccaaaacaattatcaacttcttagaaaaatgtgaaaattatccagaacattggctagaaaattctcaatggatgattgaacgcattcgccataaatataactggaattcccacacaaatcaactcctgttattaacgaaaatgtttagcttttggaacttcatctatcccgaagataacgaagccagagatcgttacatggaaagtttatttcatcttctttataaacctatagctgaccatattttatcagaacatctaagcaaaatcagaaatcataattaa

SEQ ID NO: 11 - protein, 709 aa, Synechococcus elongatus
MAAQNLYILHIQTHGLLRGQNLELGRDADTGGQTKYVLELAQAQAKSPQVQQVDIITRQITDPRVSVGYSQAIEPFAPKGRIVRLPFGPKRYLRKELLWPHLYTFADAILQYLAQQKRTPTWIQAHYADAGQVGSLLSRWLNVPLIFTGHSLGRIKLKKLLEQDWPLEEIEAQFNIQQRIDAEEMTLTHADWIVASTQQEVEEQYRVYDRYNPERKLVIPPGVDTDRFRFQPLGDRGVVLQQELSRFLRDPEKPQILCLCRPAPRKNVPALVRAFGEHPWLRKKANLVLVLGSRQDINQMDRGSRQVFQEIFHLVDRYDLYGSVAYPKQHQADDVPEFYRLAAHSGGVFVNPALTEPFGLTILEAGSCGVPVVATHDGGPQEILKHCDFGTLVDVSRPANIATALATLLSDRDLWQCYHRNGIEKVPAHYSWDQHVNTLFERMETVALPRRRAVSFVRSRKRLIDAKRLVVSDIDNTLLGDRQGLENLMTYLDQYRDHFAFGIATGRRLDSAQEVLKEWGVPSPNFWVTSVGSEIHYGTDAEPDISWEKHINRNWNPQRIRAVMAQLPFLELQPEEDQTPFKVSFFVRDRHETVLREVRQHLRRHRLRLKSIYSHQEFLDILPLAASKGDAIRHLSLRWRIPLENILVAGDSGNDEEMLKGHNLGVVVGNYSPELEPLRSYERVYFAEGHYANGILEALKHYRFFEAIA

SEQ ID NO: 12 - DNA, 2130 bp, Synechococcus elongatus
gtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaa

SEQ ID NO: 13 - DNA, 4186 bp, Artificial Sequence
  Description of Artificial Sequence Synthetic polynucleotide
caattgccctaagacagttgtcgtctttcgaagtctagttaacattaggggcgattctttgtttccactgagtggaagcaaacggtatcaaggttgcaggcagactcaaggtctagattgcttcacagcttgtgtggctatatttattatcttcattattgatggtagttgtgggtggatttaagatggaaaagtaacagataaatgtcgtctttaagggcgatctagatcgtatcgtttttaattcctaggtcggcatttattaatcaacctcgatacaatatttttttgtaaaaacttctagataaatgactcaagtctcattgaaagtctggggtgttgcctccccagtcaattcaagattaccaaggcctcgcatcgcctcttctattttgtttgaaggggacctaacgtgttgcgccaagctagttctcgacagagcatctcaagagcgcgttgctcgcggggggcaaacagttggagatcagccagctcttgcaagacttgttgggtgaggtggctggcaaagctaccggcatagcgcagtaagagactgtagtagcgaaattgcggttggccgttgcaatcgcggtgaaaggcagcaatttctgcttcgctgaggcagtagacaggggtattgaccggcacgatcgcgcgaggaatgccctgttcgccgtagcgatcgccgccctctgtttccgctaagctgccgatcaaaaccgtggagagcagcgtgcgggtttcattgattaaatcaggcgtgaatagtgggtcgggcccacttgaaagacgcggcccgttgttcagaaagaggggattaaacaactgcgagttgtagaccactccgatcgcccaatggcgatcgccttccaaacgaacaaagctgccaaatccatagctctcggcggctggtggattggagacatccatgtcgtcatccacttggacaacgtagtcacagtgcgagttggatttgacaactttgccgaggcgcatggtgctgccagtgttacaaccaattaaccaattctgacatatggacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtttcactggtgaaaagaaaaaccaccctggcgcccaatacgcaaaccgcctctccccgcgcgttggccgattcattaatgcagctggcacgacaggtttcccgactggaaagcgggcagtgagctttgttgacaattaatcatccggctcgtataatgtgtggaattgtgagcggataacaagaaggagatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaattgttcagaacgctcggtcttgcacaccgggcgttttttctttgtgagtccaggtaccaatcaatctcccccaagtcaagcggcgctgagacccagtgtctgccggtgagtcagtcttggcaagcaaactgtgcctttgcgatttcttaccctacgcagctccgggatcgatcggaggtaaccaaggctacggacaatggcgcgggcaccagctgttggtaaactgaggagcgatcgccgcttcagtccaaaggctatgacgcaaaaatccgttgttattgctccgtccattctgtcagcggatttcagccgcttgggcgacgatgtccgcgctgttgaccaggctggcgctgactggattcacgtcgatgtgatggatggtcgcttcgtccctaacatcaccattggaccgctgatcgttgaagcgctgcgcccggtgacccaaaagccgttggacgtccacttgatgatcgtcgagccggaaaaatatgtgccggatttcgcgaaagcaggggctgacatcatctcggtccaagcagaagcttgcccccacctgcaccgcaacttggctcagatcaaagacctcggcaagcaagcaggcgtcgtcctcaacccctctaccccagtcgaaaccctggaatacgtgctggagttgtgcgacctgattttgatcatgagcgtcaaccctggcttcggtggtcagagtttcatcccagctgtcctgccgaaaatccgtaagctgcgcgccatgtgcgatgagcgtggccttgatccttggattgaagtcgatggcggcttgaaagccaataacacttggcaggtgctggaagccggtgctaacgcaattgtggcgggctcggcagtcttcaacgcgccggactatgctgaagcgatcgcggcgattcgcaacagcaagcgtcctgaacttgtcactgcttaggcttctcgctcaacgctcagtggagcaatctgaatcttgcagcccttcagtggatcagtctgctgaggggttttgctttaggatgggcgatcgcgagtagggacacggatcgctggta

SEQ ID NO: 14 - DNA, 6156 bp, Saccharomyces cerevisiae
atggacgcttactctacccgtccgctgaccctgtctcacggttctctggaacacgttctgctggttccgaccgcttctttcttcatcgcttctcagctgcaggaacagttcaacaaaatcctgccggaaccgaccgaaggtttcgctgctgacgacgaaccgaccaccccggctgaactggttggtaaattcctgggttacgtttcttctctggttgaaccgtctaaagttggtcagttcgaccaggttctgaacctgtgcctgaccgaattcgaaaactgctacctggaaggtaacgacatccacgctctggctgctaaactgctgcaggaaaacgacaccaccctggttaaaaccaaagaactgatcaaaaactacatcaccgctcgtatcatggctaaacgtccgttcgacaaaaaatctaactctgctctgttccgtgctgttggtgaaggtaacgctcagctggttgctatcttcggtggtcagggtaacaccgacgactacttcgaagaactgcgtgacctgtaccagacctaccacgttctggttggtgacctgatcaaattctctgctgaaaccctgtctgaactgatccgtaccaccctggacgctgaaaaagttttcacccagggtctgaacatcctggaatggctggaaaacccgtctaacaccccggacaaagactacctgctgtctatcccgatctcttgcccgctgatcggtgttatccagctggctcactacgttgttaccgctaaactgctgggtttcaccccgggtgaactgcgttcttacctgaaaggtgctaccggtcactctcagggtctggttaccgctgttgctatcgctgaaaccgactcttgggaatctttcttcgtttctgttcgtaaagctatcaccgttctgttcttcatcggtgttcgttgctacgaagcttacccgaacacctctctgccgccgtctatcctggaagactctctggaaaacaacgaaggtgttccgtctccgatgctgtctatctctaacctgacccaggaacaggttcaggactacgttaacaaaaccaactctcacctgccggctggtaaacaggttgaaatctctctggttaacggtgctaaaaacctggttgtttctggtccgccgcagtctctgtacggtctgaacctgaccctgcgtaaagctaaagctccgtctggtctggaccagtctcgtatcccgttctctgaacgtaaactgaaattctctaaccgtttcctgccggttgcttctccgttccactctcacctgctggttccggcttctgacctgatcaacaaagacctggttaaaaacaacgtttctttcaacgctaaagacatccagatcccggtttacgacaccttcgacggttctgacctgcgtgttctgtctggttctatctctgaacgtatcgttgactgcatcatccgtctgccggttaaatgggaaaccaccacccagttcaaagctacccacatcctggacttcggtccgggtggtgcttctggtctgggtgttctgacccaccgtaacaaagacggtaccggtgttcgtgttatcgttgctggtaccctggacatcaacccggacgacgactacggtttcaaacaggaaatcttcgacgttacctctaacggtctgaaaaaaaacccgaactggctggaagaataccacccgaaactgatcaaaaacaaatctggtaaaatcttcgttgaaaccaaattctctaaactgatcggtcgtccgccgctgctggttccgggtatgaccccgtgcaccgtttctccggacttcgttgctgctaccaccaacgctggttacaccatcgaactggctggtggtggttacttctctgctgctggtatgaccgctgctatcgactctgttgtttctcagatcgaaaaaggttctaccttcggtatcaacctgatctacgttaacccgttcatgctgcagtggggtatcccgctgatcaaagaactgcgttctaaaggttacccgatccagttcctgaccatcggtgctggtgttccgtctctggaagttgcttctgaatacatcgaaaccctgggtctgaaatacctgggtctgaaaccgggttctatcgacgctatctctcaggttatcaacatcgctaaagctcacccgaacttcccgatcgctctgcagtggaccggtggtcgtggtggtggtcaccactctttcgaagacgctcacaccccgatgctgcagatgtactctaaaatccgtcgtcacccgaacatcatgctgatcttcggttctggtttcggttctgctgacgacacctacccgtacctgaccggtgaatggtctaccaaattcgactacccgccgatgccgttcgacggtttcctgttcggttctcgtgttatgatcgctaaagaagttaaaacctctccggacgctaaaaaatgcatcgctgcttgcaccggtgttccggacgacaaatgggaacagacctacaaaaaaccgaccggtggtatcgttaccgttcgttctgaaatgggtgaaccgatccacaaaatcgctacccgtggtgttatgctgtggaaagaattcgacgaaaccatcttcaacctgccgaaaaacaaactggttccgaccctggaagctaaacgtgactacatcatctctcgtctgaacgctgacttccagaaaccgtggttcgctaccgttaacggtcaggctcgtgacctggctaccatgacctacgaagaagttgctaaacgtctggttgaactgatgttcatccgttctaccaactcttggttcgacgttacctggcgtaccttcaccggtgacttcctgcgtcgtgttgaagaacgtttcaccaaatctaaaaccctgtctctgatccagtcttactctctgctggacaaaccggacgaagctatcgaaaaagttttcaacgcttacccggctgctcgtgaacagttcctgaacgctcaggacatcgaccacttcctgtctatgtgccagaacccgatgcagaaaccggttccgttcgttccggttctggaccgtcgtttcgaaatcttcttcaaaaaagactctctgtggcagtctgaacacctggaagctgttgttgaccaggacgttcagcgtacctgcatcctgcacggtccggttgctgctcagttcaccaaagttatcgacgaaccgatcaaatctatcatggacggtatccacgacggtcacatcaaaaaactgctgcaccagtactacggtgacgacgaatctaaaatcccggctgttgaatacttcggtggtgaatctccggttgacgttcagtctcaggttgactcttcttctgtttctgaagactctgctgttttcaaagctacctcttctaccgacgaagaatcttggttcaaagctctggctggttctgaaatcaactggcgtcacgcttctttcctgtgctctttcatcacccaggacaaaatgttcgtttctaacccgatccgtaaagttttcaaaccgtctcagggtatggttgttgaaatctctaacggtaacacctcttctaaaaccgttgttaccctgtctgaaccggttcagggtgaactgaaaccgaccgttatcctgaaactgctgaaagaaaacatcatccagatggaaatgatcgaaaaccgtaccatggacggtaaaccggtttctctgccgctgctgtacaacttcaacccggacaacggtttcgctccgatctctgaagttatggaagaccgtaaccagcgtatcaaagaaatgtactggaaactgtggatcgacgaaccgttcaacctggacttcgacccgcgtgacgttatcaaaggtaaagacttcgaaatcaccgctaaagaagtttacgacttcacccacgctgttggtaacaactgcgaagacttcgtttctcgtccggaccgtaccatgctggctccgatggacttcgctatcgttgttggttggcgtgctatcatcaaagctatcttcccgaacaccgttgacggtgacctgctgaaactggttcacctgtctaacggttacaaaatgatcccgggtgctaaaccgctgcaggttggtgacgttgtttctaccaccgctgttatcgaatctgttgttaaccagccgaccggtaaaatcgttgacgttgttggtaccctgtctcgtaacggtaaaccggttatggaagttacctcttctttcttctaccgtggtaactacaccgacttcgaaaacaccttccagaaaaccgttgaaccggtttaccagatgcacatcaaaacctctaaagacatcgctgttctgcgttctaaagaatggttccagctggacgacgaagacttcgacctgctgaacaaaaccctgaccttcgaaaccgaaaccgaagttaccttcaaaaacgctaacatcttctcttctgttaaatgcttcggtccgatcaaagttgaactgccgaccaaagaaaccgttgaaatcggtatcgttgactacgaagctggtgcttctcacggtaacccggttgttgacttcctgaaacgtaacggttctaccctggaacagaaagttaacctggaaaacccgatcccgatcgctgttctggactcttacaccccgtctaccaacgaaccgtacgctcgtgtttctggtgacctgaacccgatccacgtttctcgtcacttcgcttcttacgctaacctgccgggtaccatcacccacggtatgttctcttctgcttctgttcgtgctctgatcgaaaactgggctgctgactctgtttcttctcgtgttcgtggttacacctgccagttcgttgacatggttctgccgaacaccgctctgaaaacctctatccagcacgttggtatgatcaacggtcgtaaactgatcaaattcgaaacccgtaacgaagacgacgttgttgttctgaccggtgaagctgaaatcgaacagccggttaccaccttcgttttcaccggtcagggttctcaggaacagggtatgggtatggacctgtacaaaacctctaaagctgctcaggacgtttggaaccgtgctgacaaccacttcaaagacacctacggtttctctatcctggacatcgttatcaacaacccggttaacctgaccatccacttcggtggtgaaaaaggtaaacgtatccgtgaaaactactctgctatgatcttcgaaaccatcgttgacggtaaactgaaaaccgaaaaaatcttcaaagaaatcaacgaacactctacctcttacaccttccgttctgaaaaaggtctgctgtctgctacccagttcacccagccggctctgaccctgatggaaaaagctgctttcgaagacctgaaatctaaaggtctgatcccggctgacgctaccttcgctggtcactctctgggtgaatacgctgctctggcttctctggctgacgttatgtctatcgaatctctggttgaagttgttttctaccgtggtatgaccatgcaggttgctgttccgcgtgacgaactgggtcgttctaactacggtatgatcgctatcaacccgggtcgtgttgctgcttctttctctcaggaagctctgcagtacgttgttgaacgtgttggtaaacgtaccggttggctggttgaaatcgttaactacaacgttgaaaaccagcagtacgttgctgctggtgacctgcgtgctctggacaccgttaccaacgttctgaacttcatcaaactgcagaaaatcgacatcatcgaactgcagaaatctctgtctctggaagaagttgaaggtcacctgttcgaaatcatcgacgaagcttctaaaaaatctgctgttaaaccgcgtccgctgaaactggaacgtggtttcgcttgcatcccgctggttggtatctctgttccgttccactctacctacctgatgaacggtgttaaaccgttcaaatctttcctgaaaaaaaacatcatcaaagaaaacgttaaagttgctcgtctggctggtaaatacatcccgaacctgaccgctaaaccgttccaggttaccaaagaatacttccaggacgtttacgacctgaccggttctgaaccgatcaaagaaatcatcgacaactgggaaaaatacgaacagtcttaa

SEQ ID NO: 15 - DNA, 522 bp, Arabidopsis thaliana
agctctactcccattattatcgttttctgtgatcttttctacatactgtgcttcaaaagaaaaaggaaaatgcagcgtgcgttccttcgtcgtctcagcaagcgtgccgtcgttcctgcaactgcggcgctgcttcggttttctcagacgcagtgcgctggtcgtgcccctgttaccgcgtgcgcaggcgctgttcttgcctaccagtgctctatccgagcctactccgatgcccaccacgaggagagcgctactcgcagcggccaatacctcctcgacaagaacgacgtgctgacgcgtgtgctcgaggtagtgaagaacttcgagaaggttgatgcctctaaggtgacgcctgagtctcacttcgtgaacgatctcggcctcaactctctcgacgttgtggaggtcgtttttgccatcgagcaggagttcatcttagatatccctgatcacgatgccgaaaagatccagtccattcctgatgctgtggagtacattgcgcagaatccaatggccaagtaa

SEQ ID NO: 16 - DNA, 1266 bp, Leptomonas pyrrhocoris
atggaaggttctccggttacctctgttcgtctgtcttctgttgttccggcttctgttgttggtgaaaacaaaccgcgtcagctgaccccgatggacctggctatgaaactgcactacgttcgtgctgtttacttcttcaaaggtgctcgtgacttcaccgttgctgacgttaaaaacaccatgttcaccctgcagtctctgctgcagtcttaccaccacgtttctggtcgtatccgtatgtctgacaacgacaacgacacctctgctgctgctatcccgtacatccgttgcaacgactctggtatccgtgttgttgaagctaacgttgaagaattcaccgttgaaaaatggctggaactggacgaccgttctatcgaccaccgtttcctggtttacgaccacgttctgggtccggacctgaccttctctccgctggttttcctgcagatcacccagttcaaatgcggtggtctgtgcatcggtctgtcttgggctcacatcctgggtgacgttttctctgcttctaccttcatgaaaaccctgggtcagctggtttctggtcacgctccgaccaaaccggtttacccgaaaaccccggaactgacctctcacgctcgtaacgacggtgaagctatctctatcgaaaaaatcgactctgttggtgaatactggctgctgaccaacaaatgcaaaatgggtcgtcacatcttcaacttctctctgaaccacatcgactctctgatggctaaatacaccacccgtgaccagccgttctctgaagttgacatcctgtacgctctgatctggaaatctctgctgaacatccgtggtgaaaccaacaccaacgttatcaccatctgcgaccgtaaaaaatcttctacctgctggaacgaagacctggttatctctgttgttgaaaaaaacgacgaaatggttggtatctctgaactggctgctctgatcgctggtgaaaaacgtgaagaaaacggtgctatcaaacgtatgatcgaacaggacaaaggttcttctgacttcttcacctacggtgctaacctgaccttcgttaacctggacgaaatcgacatgtacgaactggaaatcaacggtggtaaaccggacttcgttaactacaccatccacggtgttggtgacaaaggtgttgttctggttttcccgaaacagaacttcgctcgtatcgtttctgttgttatgccggaagaagacctggctaaactgaaagaagaagttaccaacatgatcatctaa

SEQ ID NO: 17 - protein, 172 aa, Escherichia coli
MVDKRESYTKEDLLASGRGELFGAKGPQLPAPNMLMMDRVVKMTETGGNFDKGYVEAELDINPDLWFFGCHFIGDPVMPGCLGLDAMWQLVGFYLGWLGGEGKGRALGVGEVKFTGQVLPTAKKVTYRIHFKRIVNRRLIMGLADGEVLVDGRLIYTASDLKVGLFQDTSAF